(19) United States
(12) Patent Application Publication
(10) Pub. No.: US 2012/0014917 A1
(43) Pub. Date: Jan. 19, 2012

(54) METHODS OF TREATING HIV PATIENTS WITH ANTI-FIBROTICS

(75) Inventors: Karl Kossen, Brisbane, CA (US); Timothy Schacker, St. Paul, MN (US); Ashley T. Haase, North Oaks, MN (US)

(73) Assignees: University of Minnesota, Minneapolis, MN (US); INTERMUNE, INC., Brisbane, CA (US)

(21) Appl. No.: 13/244,752

(22) Filed: Sep. 26, 2011

Related U.S. Application Data
(63) Continuation of application No. PCT/US10/35042, filed on May 15, 2010.
(60) Provisional application No. 61/178,786, filed on May 15, 2009.

(51) Int. Cl. A61K 38/21; A61K 31/506; A61K 31/444; A61K 31/53; A61P 31/18; A61K 31/4436; A61K 31/497; A61K 31/437; A61K 31/5377; A61K 31/4418; A61K 31/513

(52) U.S. Cl. 424/85.6; 514/345; 514/256; 514/334; 514/241; 514/274; 514/336; 514/253.04; 514/300; 514/234.5

(57) ABSTRACT

The invention relates to methods of treating patients infected with human immunodeficiency virus (HIV) with a therapeutic that has anti-fibrotic effects, for example, pirfenidone and analogs thereof.

### CROSS-REFERENCE TO RELATED APPLICATIONS

This application is a continuation of International Application Number PCT/US2010/35042, filed May 15, 2010, which claims the priority benefit under 35 U.S.C. §119(e) of U.S. Provisional Application No. 61/178,786, filed May 15, 2009, each of which is incorporated herein by reference in their entirety.

### STATEMENT OF GOVERNMENT INTEREST

This invention was made with government support under Grant Numbers K24AI056986, R01AI054232, and R56AI054232, awarded by the National Institutes of Health. The government has certain rights in the invention.

### FIELD OF THE INVENTION

The invention relates to methods of treating patients infected with human immunodeficiency virus (HIV) with a therapeutic agent that has an anti-fibrotic effect, for example, pirfenidone or a derivative thereof.

### BACKGROUND

As a consequence of CD4⁺ T cell depletion, individuals infected with the human immunodeficiency virus (HIV), the causative agent of the acquired immune deficiency syndrome (AIDS) eventually succumb to opportunistic infections and malignancies if they do not receive antiretroviral therapy (ART). The World Health Organization estimates 25 million people have already died from AIDS since it was first recognized 25 years ago and that more than 32 million people are currently living with HIV-1 infection [(UNAIDS), J.U.N.P.o.H.A. 2006, 2006 report on the global AIDS epidemic].

Inhibiting viral replication with ART and reconstituting immunity, measured by increases in peripheral blood CD4+ T cells, has had great impact on this terrible morbidity and mortality of HIV-1 infection. Patients are living longer, healthier lives and mortality in the treated population of HIV+ patients has significantly declined. However, up to 20% of treated individuals receive no clinical benefit because, despite suppression of replicating virus in plasma, immune reconstitution is limited or absent [Martin et al., 2001, Eur J Clin Microbiol Infect Dis 20:871-9; Gea-Banacloche et al., 1999, AIDS13(Suppl. A):S25-38.]. Furthermore, even among patients with significant increases in peripheral blood CD4+ T cells, few reconstitute to normal levels. While the data are clear that significant increases may be sufficient to avert opportunistic infections, there is increasing recognition that these individuals may still be at risk for complications of a subtler kind of immune suppression. Recent data indeed suggest that rates of malignancy appear to be increasing in the ART-treated HIV+ population, even among those with significant reconstitution [Barbaro et al., 2007, Oncol Rep 17:1121-6; Engels et al., 2006, J Clin Oncol 24:1383-8; Grulich et al., 2007, Lancet 370:59-67; Lewden et al., 2005, Int J Epidemiol 34:121-30; Palefsky et al., 2005, AIDS 19:1407-14].

Significant increases in peripheral blood CD4⁺ T cell counts with ART have been extensively documented [O'Brien et al., 1996, New Engl J Med 334:426-31; Martin et al., 2001, Clin Microbiol Infect 7:678-81; Egger et al. 2002, Lancet 360:119-29]. However, the dynamics and extent of CD4⁺ cell depletion and reconstitution during treatment may be substantially different in secondary lymphatic tissues (LTs) and gut-associated lymphatic tissue (GALT), which collectively contain most CD4⁺ cells (98%). GALT suffers greater losses of CD4⁺ cells, compared with peripheral blood, in both simian immunodeficiency virus (SIV) infections and HIV infections; restoration in GALT, compared with that in the blood, is slow and incomplete when treatment is initiated in the chronic stage of infection [Veazey et al., 1998, Science 280: 427-31; Guadalupe et al., 2003, J Virol 77: 11708-17; Brenchley et al., 2004, J Exp Med 200: 749-59; Mehandru et al., 2004, J Exp Med 200:761-70; Mehandru et al., 2006, PLoS Med 3: e484]. Even if treatment is initiated in the early stages of infection, it is not clear whether it substantially increases restoration of CD4⁺ cells in the gut. There have been reports of increases of gut CD4⁺ cells [Talal et al., 2001, J Acquir Immune Defic Syndr 26:1-7; George 2005, J Virol 79: 2709-19], albeit delayed compared with the increases in peripheral blood, but even after 1-7 years of ART most patients continue to have substantial depletion (50%-60%) of gut lamina propria lymphocytes [Mehandru et al., 2007, J Virol 81:599-612].

It is an object of the present invention to provide novel therapies and therapeutic regimens for treating patients infected with HIV.

### SUMMARY OF THE INVENTION

Without being bound by any particular theory of the invention, fibrosis in lymphatic tissues in response to HIV infection is believed to result in depletion and relatively limited reconstitution of CD4⁺ cells in lymphatic tissue and that the extent of collagen deposition in the lymphatic tissue is correlated with the extent of depletion and impaired reconstitution of CD4⁺ cells in lymphatic tissue. The results described herein indicate that the use of an anti-fibrotic agent can provide measurable beneficial effects in the extent of collagen deposition in lymphatic tissue and the size of the CD4⁺ T cell population in patients infected with HIV.

Accordingly, one aspect of the invention provides method of treatment including administering a therapeutically effective amount of an anti-fibrotic agent and a HIV therapeutic agent to a patient that is diagnosed with HIV. In another aspect, provided herein is a method of treating a patient diagnosed with HIV and receiving an HIV therapeutic agent, the improvement comprising further administering to the patient an anti-fibrotic agent.

The amount of the HIV therapeutic agent administered can be a reduced amount relative to the amount which would be administered to a patient diagnosed with HIV in the absence of the anti-fibrotic agent, or the amount of the HIV therapeutic agent can be the same amount which would administered to a patient diagnosed with HIV in the absence of the anti-fibrotic agent.

The administering of the anti-fibrotic agent can commence when the patient has a T cell count of at least about 350 cells per mm³ in the absence of treatment with a HIV therapeutic agent. In related aspects, the administration can commence when the patient diagnosed with HIV has a T cell count of at least about 400 cells per mm³, or at least about 450 cells per mm³, or at least about 500 cells per mm³ or greater. In further aspects, the administration can commence when the patient diagnosed with HIV has a T cell count of less than about 350 cells per mm³. In related aspects, the administration can commence when the patient diagnosed with HIV has a T cell count of less than about 300 cells per mm³, or less than about 250 cells per mm³, or less than about 200 cells per mm³, or less than about 150 cells per mm³, or less than about 100 cells per mm³ or fewer.

A further embodiment of the invention provides a method of treatment including administering a therapeutically effective amount of an anti-fibrotic agent to a patient diagnosed with HIV prior to administering a therapeutically effective amount of a HIV therapeutic agent to the patient diagnosed with HIV. In one aspect of this embodiment, the administration of the anti-fibrotic agent can commence when the patient diagnosed with HIV has a T cell count of at least about 350 cells per mm³. In other embodiments, the administration can commence when the patient diagnosed with HIV has a T cell count of at least about 400 cells per mm³, or at least about 450 cells per mm³, or at least about 500 cells per mm³ or greater. In further aspects, the administration can commence when the patient diagnosed with HIV has a T cell count of less than about 350 cells per mm³. In related aspects, the administration can commence when the patient diagnosed with HIV has a T cell count of less than about 300 cells per mm³, or less than about 250 cells per mm³, or less than about 200 cells per mm³, or less than about 150 cells per mm³, or less than about 100 cells per mm³ or fewer. In other aspects, the patient is one who is a pregnant woman, one with HIV-associated nephropathy, or one who is coinfected with hepatitis B virus (HBV) when treatment of HBV is indicated.

Administration of the HIV therapeutic agent, in some embodiments, commences while the patient diagnosed with HIV has a T cell count of less than 350 cells per mm³. In related aspects, the administration can commence when the patient diagnosed with HIV has a T cell count of less than about 300 cells per mm³, or less than about 250 cells per mm³, or less than about 200 cells per mm³, or less than about 150 cells per mm³, or less than about 100 cells per mm³ or fewer.

With respect to administration of the anti-fibrotic agent and the HIV therapeutic agent, the methods disclosed herein contemplate, in various aspects, the commencement of either one of or both of the anti-fibrotic agent and the HIV therapeutic agent to be related to the T cell count of the patient diagnosed with HIV. For example, in some embodiments, administration of the anti-fibrotic agent commences while the patient diagnosed with HIV has a T cell count that is, for example, at least about 350 cells per mm³. In another embodiment, administration of the anti-fibrotic agent commences while the patient diagnosed with HIV has a T cell count that is, for example, at least about 350 cells per mm³ and administration of the HIV therapeutic agent commences while the patient diagnosed with HIV has a T cell count that is, for example, less than about 350 cells per mm³. In a further embodiment, the anti-fibrotic agent is co-administered with the HIV therapeutic agent while the patient diagnosed with HIV has a T cell count that is, for example, at least about 350 cells per mm³. In still further embodiments, the anti-fibrotic agent is co-administered with the HIV therapeutic agent while the patient diagnosed with HIV has a T cell count that is, for example, less than about 350 cells per mm³. In alternatives, the administration of either agent can be commenced according to the other thresholds disclosed herein.

In any of the methods described herein, the HIV therapeutic agent preferably is selected from the group consisting of a nucleoside reverse transcriptase inhibitor, a non-nucleoside reverse transcriptase inhibitor, a protease inhibitor, a CCR5 antagonist an integrase inhibitor, a fusion inhibitor, and combinations thereof, although other HIV therapeutic agents can be selected by a person of ordinary skill in the art.

Methods according to the invention are disclosed herein to be effective at reducing the median percent area of a lymphatic tissue T cell zone occupied by collagen in a patient diagnosed with HIV relative to a patient diagnosed with HIV that was not administered an anti-fibrotic agent. A method described herein preferably will reduce the median percent area of a lymphatic tissue T cell zone occupied by collagen in a patient diagnosed with HIV relative to a patient diagnosed with HIV that was not administered an anti-fibrotic agent by at least 5%, at least 10%, at least 15%, at least 20%, at least 25%, at least 30%, at least 35%, at least 40%, at least 45%, at least 50%, at least 55%, or at least 60%. In some aspects, the reduction in median percent area of a lymphatic tissue T cell zone occupied by collagen in a patient diagnosed with HIV relative to a patient diagnosed with HIV that was not administered an anti-fibrotic agent is measured about 2 weeks after commencing the anti-fibrotic agent. In various aspects, the reduction is measured about 3 weeks, about 4 weeks, about 5 weeks, about 6 weeks, about 7 weeks, about 8 weeks, about 9 weeks, about 10 weeks, about 12 weeks, about 14 weeks, about 16 weeks, about 18 weeks, about 20 weeks, about 25 weeks, about 30 weeks, about 35 weeks, about 40 weeks, about 45 weeks, about 50 weeks, about 52 weeks or more after commencing the anti-fibrotic agent.

Preferably, a method described herein will also be effective at increasing the number of CD4⁺ T cells in a patient infected with HIV relative to a patient infected with HIV that was not administered an anti-fibrotic agent. In various aspects, the number of CD4⁺ T cells is increased by about 10, or about 20, or about 30, or about 40, or about 50, or about 60, or about 70, or about 80, or about 90, or about 100, or about 110, or about 120, or about 130, or about 140, or about 150, or about 160, or about 170, or about 180, or about 190, or about 200, or about 250, or about 300, or about 350, or about 400, or about 450, or about 500 cells per mm³. In some aspects, the increase in the number of CD4⁺ T cells is detected after about 1 week of administration of an anti-fibrotic agent. In various aspects, the increase is detected after about 2 weeks, about 3 weeks, about 4 weeks, about 5 weeks, about 6 weeks, about 7 weeks, about 8 weeks, about 9 weeks, about 10 weeks, about 12 weeks, about 14 weeks, about 16 weeks, about 18 weeks, about 20 weeks, about 25 weeks, about 30 weeks, about 35 weeks, about 40 weeks, about 45 weeks, about 50 weeks, about 52 weeks or more of administration of an anti-fibrotic agent.

In one embodiment, practice of the methods of the invention is effective to preserve or maintain a patient's ability to increase the number of CD4⁺ cells in a patient that is administered anti-fibrotic therapy to an extent greater than what would be possible in the absence of treatment according to the method (e.g., in the absence of anti-fibrotic therapy). For example, the increase in the number of CD4⁺ cells may not be realized while being administered the anti-fibrotic agent, but would be realized during treatment with an HIV therapeutic, e.g. ART. It is contemplated that preservation of the patient's ability to increase the number of CD4⁺ cells during subsequent treatment with a HIV therapeutic would result in a greater increase and/or a more prolonged increase in the number of CD4⁺ cells, as compared to a patient who did not receive anti-fibrotic therapy. In related aspects, administration of the anti-fibrotic agent to a patient diagnosed with HIV according to a method disclosed herein results in the prevention of a decrease in CD4⁺ T cells relative to a patient diagnosed with HIV that does not receive anti-fibrotic treatment.

Administration of the anti-fibrotic agent to a patient diagnosed with HIV according to a method disclosed herein preferably results in the attenuation of the rate of collagen deposition in lymphatic tissue relative to patients diagnosed with HIV that do not receive anti-fibrotic treatment. More preferably, administration of the anti-fibrotic agent to a patient diagnosed with HIV according to a method disclosed herein results in the prevention of an increase in collagen in lymphatic tissue relative to patients diagnosed with HIV that do not receive anti-fibrotic treatment.

In some embodiments, the anti-fibrotic agent is pirfenidone or a pirfenidone analog. Accordingly, in an embodiment, a method of treating a patient diagnosed with HIV is provided comprising administering to the patient a therapeutically effective amount of pirfenidone or a pirfenidone analog, optionally in combination with a therapeutically effective amount of a HIV therapeutic agent.

In another embodiment, administration of pirfenidone or a pirfenidone analog is alternated in periods of time with administration of the HIV therapeutic agent. For example, the alternating administration can be performed due to a contraindication of administration of the HIV therapeutic agent with pirfenidone or a pirfenidone analog. In addition or in the alternative, the contraindication can be selected from the group consisting of an adverse drug interaction, HIV resistance to a HIV therapeutic agent, and combinations thereof. Thus, anti-fibrotic administration is continued while the HIV therapy is discontinued, in order to impede fibrosis of lymph tissue and preserve lymph node function.

Administration of pirfenidone or a pirfenidone analog preferably is commenced at the time the patient is diagnosed with HIV. Administration of pirfenidone or a pirfenidone analog can be stopped when contraindicated. For example, the contraindication can be selected from the group consisting of an adverse drug interaction, HIV resistance to a HIV therapeutic agent, and combinations thereof.

In an embodiment, a method of treating a patient diagnosed with HIV is provided comprising administering to said patient a therapeutically effective amount of pirfenidone or a pirfenidone analog in the absence of a HIV therapeutic agent for a first period of time; and administering a therapeutically effective amount of HIV therapeutic agent in the absence of administration of pirfenidone or a pirfenidone analog (i.e., in the absence of administration of pirfenidone and in the absence of administration of pirfenidone analogs) for a second period of time following the first period of time. Optionally, an anti-fibrotic agent can be administered in combination with a HIV therapeutic agent for a third period of time following the first period of time and prior to the second period of time. In one aspect, the first period of time is one week. In related aspects, the first period of time can be about 2, 3, 4, 5, 6, 7, 8, 9, 10, 12, 14, 16, 18, 20, 25, 30, 35, 40, 45, 50, or about 52 weeks. In another aspect, the third period of time is one week. In related aspects, the third period of time can be about 2, 3, 4, 5, 6, 7, 8, 9, 10, 12, 14, 16, 18, 20, 25, 30, 35, 40, 45, 50, or about 52 weeks. In another aspect, the second period of time is one day. In related aspects, the second period of time can be 2, 3, 4, 5, 6, or 7 days. In further aspects, the second period of time can be about 2, 3, 4, 5, 6, 7, 8, 9, 10, 12, 14, 16, 18, 20, 25, 30, 35, 40, 45, 50, or about 52 weeks. In still further aspects, the second period of time can be about 2, 3, 4, 5, 6, 7, 8, 9, 10, 12, 14, 16, 18, 20, 25, 30, 35, 40, 45, 50 or more years.

As described above, in any of the methods described herein, the patient diagnosed with HIV can be treated with an anti-fibrotic agent prior to treatment with an HIV therapeutic agent. In the alternative, in any of the methods described herein the patient diagnosed with HIV can be one who has previously been treated with an HIV therapeutic agent. In yet a further alternative, in any of the methods described herein the patient diagnosed with HIV can be one that failed to respond to prior antiretroviral therapy.

The therapeutic agent having an anti-fibrotic effect can be combined with a pharmaceutically acceptable carrier according to any principle of pharmaceutics. The HIV therapeutic agent can be combined with the same pharmaceutically acceptable carrier together with the anti-fibrotic agent, or with the same or a different pharmaceutically acceptable carrier in a separate dosage form. The route of administration preferably is oral.

The therapeutically effective amount of pirfenidone, for example, preferably is a total daily dose in a range of about 50 mg to about 4800 mg, or in a range of about 50 mg to about 2400 mg.

In embodiments, the therapeutically effective amount of the anti-fibrotic agent is administered in divided doses, e.g. three times a day or two times a day. In the alternative, is the therapeutically effective amount of the anti-fibrotic agent can be administered in a single dose once a day.

The anti-fibrotic therapeutic preferably therapeutic is pirfenidone or compound of formula (I), (II), (III), (IV), or (V) or a pharmaceutically acceptable salt, ester, solvate, or prodrug thereof:

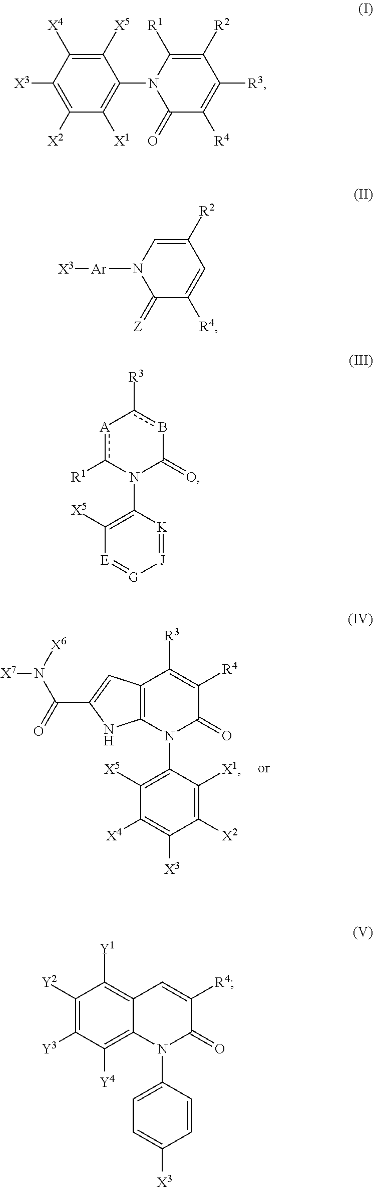

wherein

A is N or CR²; B is N or CR⁴; E is Nor CX⁴; G is N or CX³; J is N or CX²; K is N or CX¹; a dashed line is a single or double bond,

R¹, R², R³, R⁴, X¹, X², X³, X⁴, X⁵, Y¹, Y², Y³, and Y⁴ are independently selected from the group consisting of H, deuterium, C₁-C₁₀ alkyl, C₁-C₁₀ deuterated alkyl, substituted C₁-C₁₀ alkyl, C₁-C₁₀ alkenyl, substituted C₁-C₁₀ alkenyl, C₁-C₁₀ thioalkyl, C₁-C₁₀ alkoxy, substituted C₁-C₁₀ alkoxy, cycloalkyl, substituted cycloalkyl, heterocycloalkyl, substituted heterocycloalkyl, heteroalkyl, substituted heteroalkyl, aryl, substituted aryl, heteroaryl, substituted heteroaryl, halogen, hydroxyl, C₁-C₁₀ alkoxyalkyl, substituted C₁-C₁₀ alkoxyalkyl, C₁-C₁₀ carboxy, substituted C₁-C₁₀ carboxy, C₁-C₁₀ alkoxycarbonyl, substituted C₁-C₁₀ alkoxycarbonyl, CO-uronide, CO-monosaccharide, CO-oligosaccharide, and CO-polysaccharide;

X⁶ and X⁷ are independently selected from the group consisting of hydrogen, aryl, substituted aryl, heteroaryl, substituted heteroaryl, cycloalkyl, substituted cycloalkyl, heterocycloalkyl, substituted heterocycloalkyl, alkylenylaryl, alkylenylheteroaryl, alkylenylheterocycloalkyl, alkylenylcycloalkyl, or X⁶ and X⁷ together form an optionally substituted 5 or 6 membered heterocyclic ring; and

Ar is pyridinyl or phenyl; and Z is O or S.

In some embodiments, A is N or CR²; B is N or CR⁴; E is N, N⁺X⁴ or CX⁴; G is N, N⁺X³ or CX³; J is N, N⁺X² or CX²; K is N, N⁺X¹ or CX¹; a dashed line is a single or double bond,

R¹, R², R³, R⁴, X¹, X², X³, X⁴, X⁵, Y¹, Y², Y³, and Y⁴ are independently selected from the group consisting of H, deuterium, optionally substituted C₁-C₁₀ alkyl, optionally substituted C₁-C₁₀ deuterated alkyl, optionally substituted C₁-C₁₀ alkenyl, optionally substituted C₁-C₁₀ thioalkyl, optionally substituted C₁-C₁₀ alkoxy, optionally substituted cycloalkyl, optionally substituted heterocycloalkyl, optionally substituted heteroalkyl, optionally substituted aryl, optionally substituted heteroaryl, optionally substituted amido, optionally substituted sulfonyl, optionally substituted amino, optionally substituted sulfonamido, optionally substituted sulfoxyl, cyano, nitro, halogen, hydroxyl, SO₂H₂, optionally substituted C₁-C₁₀ alkoxyalkyl, optionally substituted C₁-C₁₀ carboxy, optionally substituted C₁-C₁₀ alkoxycarbonyl, CO-uronide, CO-monosaccharide, CO-oligosaccharide, and CO-polysaccharide;

X⁶ and X⁷ are independently selected from the group consisting of hydrogen, optionally substituted aryl, optionally substituted heteroaryl, optionally substituted cycloalkyl, optionally substituted heterocycloalkyl, optionally substituted alkylenylaryl, optionally substituted alkylenylheteroaryl, optionally substituted alkylenylheterocycloalkyl, optionally substituted alkylenylcycloalkyl, or X⁶ and X⁷ together form an optionally substituted 5 or 6 membered heterocyclic ring; and

Ar is optionally substituted pyridinyl or optionally substituted phenyl; and Z is O or S.

Preferably, the anti-fibrotic agent is pirfenidone, a pirfenidone derivative described herein, or a pharmaceutically acceptable salt, ester, solvate (including hydrates), or prodrug of any of the foregoing.

The anti-fibrotic therapeutic agent administered to said patient can comprise a compound of formula (II)

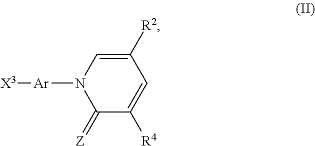

wherein

X³ is H, OH, or C₁₋₁₀alkoxy, Z is O, and R² is methyl, C(═O)H, C(═O)CH₃, C(═O)O-glucosyl, fluoromethyl, difluoromethyl, trifluoromethyl, methylmethoxyl, methylhydroxyl, or phenyl; and R⁴ is H or hydroxyl,

or a salt, ester, solvate, or prodrug thereof.

The anti-fibrotic therapeutic agent administered to said patient can be selected from the group consisting of

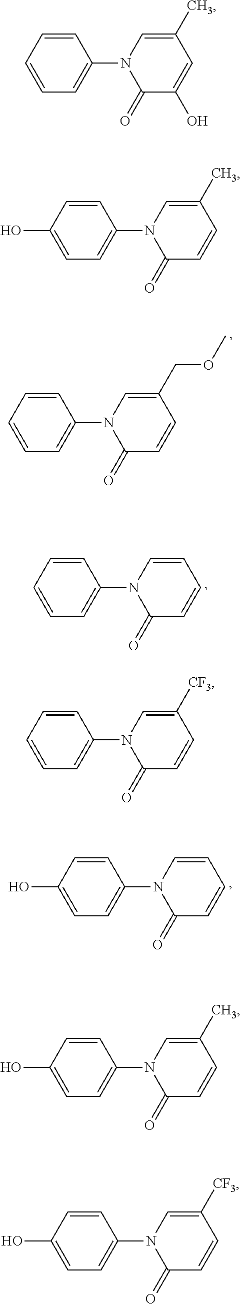

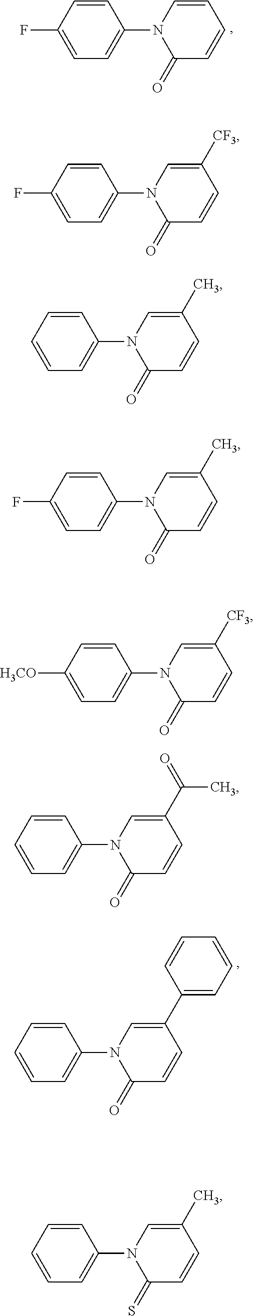

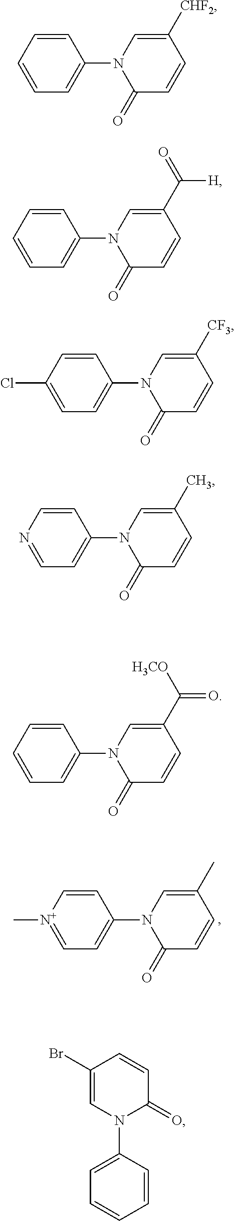

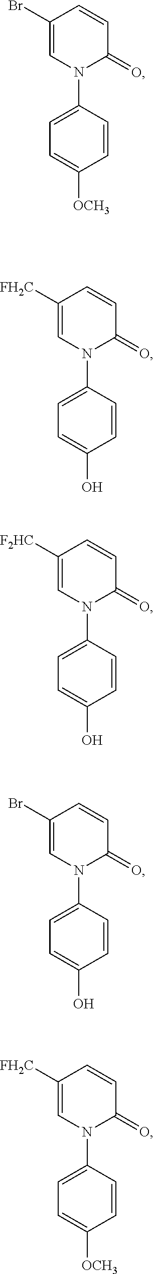

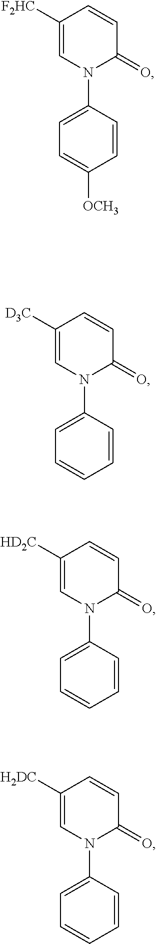

a compound as listed in Table 1, below,
and pharmaceutically acceptable salts, esters, solvates, and prodrugs thereof.

In embodiments, the patient can be human.

### BRIEF DESCRIPTION OF THE DRAWINGS

FIG. 1 depicts an outline of the regimen employed in a pilot study to determine potential for anti-fibrotic effect of pirfenidone in a non-human primate model.

FIG. 2 depicts the quantitation in lymph node and rectal biopsy samples of naïve versus central memory (T_{CM}) T cells in pirfenidone-treated versus non-treated animals.

FIG. 3 depicts changes in peripheral blood CD4 T cell count (A), CD4% (B) and plasma SIV RNA load (C) and are plotted against time. Animals with the ID beginning with AZ are treated with pirfenidone (200 mg/kg BID) and those beginning with AY are not. AZ06 appears to be an “elite controller” and has lower plasma viral loads than the other animals. AY25 is now deceased and appears to have been a “rapid progressor” with consistently high levels of replicating virus. There was no obvious difference in CD4 count, CD4%, or viral load in peripheral blood between the two groups.

FIG. 4 shows the measures of fibrosis (A, C, and E) and the size of the CD4⁺ T cell population (B, D, and F) as analyzed compared to time from infection. Tissues analyzed were lymph node (A, B), Peyer's Patch (C, D), and Lamina Propria (E, F). In lymph nodes of animals given pirfenidone have less fibrosis and higher CD4 numbers than control animals. The CD4 population is also higher in Peyer's Patches.

FIG. 5 depicts in vivo results of pirfenidone administration to monkeys to inhibit TGFβ resulting in a significant difference in terms of collagen (FIG. 5A) and the size of the T cell population (FIG. 5B).

FIG. 6 depicts a study protocol wherein pirfenidone is administered from Week −2 (relative to SIV infection) through the end of the study and co-administration of antiretroviral therapy is initiated at Week 8 and continued through the end of the study.

FIG. 7 depicts the area of T cell zone (TZ) occupied by collagen by treatment group.

FIG. 8 depicts the CD4 T Cells in TZ by treatment group.

FIG. 9 depicts the absolute naïve CD4 T Cells in TZ by treatment group.

FIG. 10 depicts the percentage of CD4 T Cells in GALT by treatment group.

FIG. 11 depicts a comparison of area staining positive for fibrosis (A&B) and CD4⁺ T cells (C&D) in the protocols described in FIG. 1 (A&C) and FIG. 6 (B&D).

FIG. 12 depicts a composite presentation of area staining positive for fibrosis (A) and area staining positive for CD4⁺ T cells (B). ARV+ Pirfenidone and ARV only groups are taken from the protocol shown in FIG. 6. Pirfenidone only and no treatment groups are taken from the protocol shown in FIG. 1.

### DETAILED DESCRIPTION OF THE INVENTION

Pirfenidone (PFD) is an orally active, anti-fibrotic agent. The invention contemplates that pirfenidone exhibits specific and potent attenuation of T cell zone fibrosis and preferably also an increase in CD4⁺ T cell populations in lymphatic tissue in patients diagnosed with HIV relative to those patients diagnosed with HIV who are not administered pirfenidone.

Pirfenidone is a small drug molecule whose chemical name is 5-methyl-1-phenyl-2-(1H)-pyridone. It is a non-peptide synthetic molecule with a molecular weight of 185.23 daltons. Its chemical elements are expressed as C₁₂H₁₁NO, and its structure and synthesis are known. Several pirfenidone Investigational New Drug Applications (INDs) have been filed with the U.S. Food and Drug Administration. Human investigations are ongoing or have been completed for pulmonary fibrosis, renal glomerulosclerosis, and liver cirrhosis. There have been other Phase II studies that used pirfenidone to attempt to treat benign prostate hypertrophy, hypertrophic scarring (keloids), and rheumatoid arthritis.

Pirfenidone is being investigated for therapeutic benefits to patients suffering from fibrosis conditions such as Hermansky-Pudlak Syndrome (HPS), associated pulmonary fibrosis and idiopathic pulmonary fibrosis (IPF). Pirfenidone is also being investigated for a pharmacologic ability to prevent or remove excessive scar tissue found in fibrosis associated with injured tissues including that of lungs, skin, joints, kidneys, prostate glands, and livers.

Pirfenidone has been reported to inhibit excessive biosynthesis or release of various cytokines such as TNF-α, TGF-β1, bFGF, PDGF, and EGF [Zhang et al., 1998, Australian and New England J Ophthalmology 26:S74-S76; Cain et al., 1998, Int'l J Immunopharmacology 20:685-695]. Pirfenidone has also been reported to decrease collagen expression and to alter the balance of matrix metalloproteinases (MMPs) and their endogenous inhibitors (tissue inhibitor of metalloproteinases or TIMPs).

### DEFINITIONS

In describing and claiming the invention, the following terminology will be used in accordance with the definitions set forth below, unless expressly indicated otherwise in context.

The acronyms “ART” and “ARV” are used interchangeably herein and mean antiretroviral therapy.

As used herein, the term “pharmaceutically acceptable carrier” includes any suitable pharmaceutically acceptable carrier, including the standard pharmaceutical carriers such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, in any one of the methods described herein the term “infected with HIV” refers to patients infected with HIV and, in the alternative, patients infected with HIV and diagnosed with HIV infection.

The salts, e.g., pharmaceutically acceptable salts, of the disclosed anti-fibrotic therapeutics may be prepared by reacting an appropriate base or acid with a stoichiometric equivalent of the therapeutic. Similarly, pharmaceutically acceptable derivatives (e.g., esters), metabolites, hydrates, solvates and prodrugs of the therapeutic may be prepared by methods generally known to those skilled in the art. Thus, another embodiment provides methods of using compounds that are prodrugs of an active compound. In general, a prodrug is a compound which is metabolized in vivo (e.g., by a metabolic transformation such as deamination, dealkylation, de-esterification, and the like) to provide an active compound. A “pharmaceutically acceptable prodrug” means a compound which is, within the scope of sound medical judgment, suitable for pharmaceutical use in a patient without undue toxicity, irritation, allergic response, and the like, and effective for the intended use, including a pharmaceutically acceptable ester as well as a zwitterionic form, where possible, of the therapeutic. As used herein, the term “pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Representative examples of particular esters include, but are not limited to, formates, acetates, propionates, butyrates, acrylates and ethylsuccinates. Examples of pharmaceutically-acceptable prodrug types are described in Higuchi and Stella, Pro-drugs as Novel Delivery Systems, Vol. 14 of the A.C.S. Symposium Series, and in Roche, ed., Bioreversible Carriers in Drug Design, American Pharmaceutical Association and Pergamon Press, 1987, both of which are incorporated herein by reference.

The compounds and compositions for use in the methods described herein may also include metabolites. As used herein, the term “metabolite” means a product of metabolism of an anti-fibrotic agent that exhibits an activity in vitro or in vivo in common with the base anti-fibrotic therapeutic. The anti-fibrotic compounds and compositions described herein may also include hydrates and solvates. As used herein, the term “solvate” refers to a complex formed by a solute (herein, the therapeutic) and a solvent. Such solvents for the purpose of the embodiments preferably should not negatively interfere with the biological activity of the solute. Solvents may be, by way of example, water, ethanol, or acetic acid.

In view of the foregoing, reference herein to a particular compound or genus of compounds will be understood to include the various forms described above, including pharmaceutically acceptable salts, esters, other prodrugs, metabolites and solvates (including hydrates) thereof.

As used herein an “effective” amount or a “therapeutically effective amount” of an anti-fibrotic agent or a HIV therapeutic agent refers to a nontoxic but sufficient amount of the agent(s) to provide the desired effect. For example one desired effect would be the reduction of T cell zone fibrosis in lymphatic tissue in a patient diagnosed with HIV relative to a patient diagnosed with HIV that was not administered the therapeutic(s). An alternative desired effect for the anti-fibrotic agent or HIV therapeutic agent of the present disclosure would include an increase in the CD4⁺ cell population in the T cell zone of lymphatic tissue in a patient diagnosed with HIV relative to a patient diagnosed with HIV that was not administered the therapeutic(s). The amount that is “effective” may vary from subject to subject, depending on the age and general condition of the individual, mode of administration, regimen for administration, and the like. Thus, it is not always possible to specify a universal “effective amount.” However, an appropriate “effective” amount in any individual case may be determined by one of ordinary skill in the art using routine experimentation.

### Human Immunodeficiency Virus (HIV)

Naïve and memory T lymphocyte numbers are maintained constant in adult animals to ensure that the organism can mount an immune response to a variety of new antigens while keeping high levels of memory cells to previously encountered pathogens [Freitas et al., 2000, Annu Rev Immunol 18:83-111; Marrack et al. 2000, Nat Immunol 1:107-12; Goldrath et al., 1999, Nature 402:255-62]. In intact animals naïve T cells divide very slowly, while memory cells have a higher rate of division [Tough et al., 1994, J Exp Med 179:1127-35]. Furthermore, in T cell deficient mice, transferred naïve T cells rapidly proliferate in the absence of antigen, to reconstitute the lymphocyte pool while undergoing a limited differentiation process [Oehen et al., 1999, Eur J Immunol 29:608-14; Murali-Krishna et al., 2000, J Immunol 165:1733-7; Goldrath et al., 2000, J Exp Med 192:557-64; Cho et al., 2000, J Exp Med 192:549-56].

Naïve and memory cells have distinct capacities to traffic in lymphoid and non-lymphoid tissues [Mackay et al., 1990, J Exp Med 171:801-17]. Evidence indicates that memory CD4⁺ and CD8⁺ T cells comprise at least two functionally distinct subsets: i) non-polarized “central memory” T cells (T_{CM}), which express CCR7 and CD62L and home to the T cell areas of secondary lymphoid organs and ii) polarized “effector memory” T cells (T_{EM}), which have lost the expression of CCR7 and have acquired the capacity to migrate to non-lymphoid tissues [Sallusto et al., 1999, Nature 401:708-12; Weninger et al., 2001, J Exp Med 194:953-66; Masopust et al., 2001, Science 291:2413-7; Reinhardt et al., 2001, Nature 410:101-5; Iezzi et al., 2001, J Exp Med 193:987-93; Geginat et al., 2003, Pathologie Biologie 51 64-66].

Human immunodeficiency virus (HIV)/AIDS is associated with profound depletion of CD4⁺ T cells in peripheral blood and throughout the secondary peripheral [Schacker et al., 2002, J. Clin. Investig. 110:1133-1139; Schacker et al., 2002, J. Infect. Dis. 186:1092-1097; Zhang et al., 1998, Proc. Natl. Acad. Sci. USA 95:1154-1159] and gut associated lymphoid tissues (GALT) [Brenchley et al., 2004, J. Exp. Med. 200:749-759; Guadalupe et al., 2003, J. Virol. 77:11708-11717; Mehandru et al., 2004, J. Exp. Med. 200:761-770; Veazey et al., 1998, Science 280:427-431], where most (98%) of these cells reside. Severe depletion occurs within 14 days of HIV acquisition (i.e. during the period of seroconversion) in the lamina propria of GALT (the effector site) and by the time the individual progresses to the chronic stage of disease >50% of CD4⁺ T cells in lymph nodes (LN) are lost [Brenchley et al., 2006, Nat Immunol 7:235-9; Brenchley et al., 2004, J Exp Med 200:749-59; Clayton et al., 1997, Clin Exp Immunol 107:288-92; Guadalupe et al., 2003, J Virol 77:11708-17; Li et al., 2005, Nature 434:1148-52; Mattapallil et al., 2005, Nature 434:1093-7; Vajdy et al., 2001, J Infect Dis 184:1007-14].

Multiple mechanisms have been proposed to explain this depletion, including decreased thymic output [Dion et al., 2004, Immunity 21:757-768; Douek et al., 2001, J. Immunol. 167:6663-6668], direct viral cytopathicity [Ahsan et al., 1998, Semin. Nephrol. 18:422-435; Cao et al., 1996, J. Virol. 70:1340-1354; Casella et al., 1997, Curr. Opin. Hematol. 4:24-31; Gandhi et al., 1998, J. Exp. Med. 187:1113-1122; Lenardo et al., 2002, J. Virol. 76:5082-5093; Stewart et al., 1997, J. Virol. 71:5579-55921, T-cell-mediated cytolysis of infected cells [McMichael et al., 2001, Nature 410:980-987], and chronic immune activation leading to increased rates of apoptosis and attrition of CD4⁺ T-cell naïve and memory pools.

Disruption of the normal T cell zone architecture by fibrosis plays an important role in the well-documented and substantial depletion of naïve CD4⁺ T cells in peripheral blood and lymphatic tissue in HIV-1 infection.

There is increasing recognition that the structure of secondary LT plays a critical role in immune system homeostasis [Kaldjian et al., 2001, Int. Immunol. 13:1243-1253; Gretz et al., 1996, J. Immunol. 157:495-499; Gretz et al., 2000, J. Exp. Med. 192:1425-1440]. These architectural elements provide an organizing structure that supports a unique microenvironment, or niche, necessary to support the immune functions of that tissue. The paracortical T cell zone (TZ), where 98% of CD4⁺ T cells in the human body normally reside [Zhang et al., 1998, Proc. Natl. Acad. Sci. USA. 95:1154-1159], is one such niche. It is organized to mount and resolve an immune response through interaction between naïve and memory T cells with antigen-presenting cells (APCs) and to provide a source of growth factors and cytokines. It is also a site for MHC class I, II, and self-peptide interactions that are necessary to maintain a normal-sized population of CD4+ and CD8+ T cells [Campbell et al., 2002, J. Exp. Med. 195:135-141; Dai et al., 2001, J. Immunol. 167:6711-6715; Dummer et al., 2001, J. Immunol. 166:2460-2468; Rathmell et al., 2001, J. Immunol. 167:6869-6876; Ploix et al., 2001, J. Immunol. 167:6724-6730].

It is within this complex TZ niche that most significant events of HIV-1 pathogenesis occur. Over 99% of virions produced during the course of infection are made in activated CD4⁺ T cells that reside in the TZ, and it is here that the pathological hallmark of HIV-1 infection, depletion of CD4⁺ T cells, is manifest and where adaptive and innate immune defenses interact in an attempt to halt viral replication [Zhang et al., 1998, Proc. Natl. Acad. Sci. USA. 95:1154-1159; Haase et al., 1996, Science. 274:985-989; Haase, 1999, Annu. Rev. Immunol. 17:625-656; Schacker et al., 2001, J. Infect. Dis. 183:555-562]. As the infection progresses, the size of the TZ is diminished and the organizing structure is lost [Pantaleo et al., 1993, AIDS. 7:S19-S23; O'Murchadha et al., 1987, Am. J. Surg. Pathol. 11:94-99; Biberfeld et al., 1985, Cancer Res. 45:4665s-4670s]. This pathological change may limit the ability of the tissue to recover normal function with antiretroviral therapy (ART).

Accordingly, as described herein, methods are provided for treating a patient who is diagnosed with HIV comprising administering to the patient a therapeutically effective amount of an anti-fibrotic agent (e.g., pirfenidone or a pirfenidone analog) and a HIV therapeutic agent, the amount of the anti-fibrotic agent effective to decrease fibrosis in lymphatic tissue relative to a patient that is not treated and the amount of the HIV therapeutic agent effective to increase CD4⁺ T cells in said patient relative to a patient that is not treated. In embodiments, the amount of HIV therapeutic agent administered to the patient can be a reduced amount relative to the amount administered to the patient in the absence of the anti-fibrotic agent.

### Reduction of Fibrosis in Lymphatic Tissue

In embodiments, methods are provided wherein a decrease in fibrosis in lymphatic tissue is achieved when an anti-fibrotic agent and a HIV therapeutic agent are administered to a patient diagnosed with HIV, the decrease relative to a patient diagnosed with HIV that is not so treated. The relative decrease in fibrosis can be at least 5%, at least 10%, at least 15%, at least 20%, at least 25%, at least 30%, at least 35%, at least 40%, at least 45%, at least 50%, at least 55%, or at least 60%. The decrease in fibrosis can be determined by methods known in the art. As an example, the following protocol may be used. Following lymphatic tissue biopsy, a 5 μm sample is cut from the baseline tissue and stained with a trichrome stain using the Masson method. Multiple (approximately 18) images are obtained from the TZ of each tissue sample and imported into PHOTOSHOP CS2 v.9.0 imaging software (ADOBE SYSTEMS INC., San Jose, Calif., USA). Image analysis tools from REINDEER GRAPHICS (Asheville, N.C.) provide high sensitivity for isolating collagen fibers to quantify the percent area occupied by collagen.

### Increase in CD4⁺ T Cells

In embodiments, methods are provided wherein an increase in CD4⁺ T cells in lymphatic tissue is contemplated when an anti-fibrotic agent and a HIV therapeutic agent is administered to a patient diagnosed with HIV, the increase relative to a patient diagnosed with HIV that is not so treated. The relative increase in CD4⁺ T cells can be at least 5%, at least 10%, at least 15%, at least 20%, at least 25%, at least 30%, at least 35%, at least 40%, at least 45%, or at least 50%. The increase in CD4⁺ T cells can be determined by methods known in the art. As an example, the following protocol can be used. Two compartments may be analyzed when quantifying CD4⁺ T cells. These are inguinal LNs and gut tissues. Tissue biopsies (LN and ileal GALT samples) are obtained and processed by immunohistochemical staining for quantitative image analysis to determine the absolute size (e.g., percent area) of the total CD4⁺ cell population.

To quantify the total CD4⁺ T cell population in each compartment, 4-μm sections are prepared from the fixed tissues and stained with antibody for CD4 by using either clone 1F6 (VENTANA MEDICAL SYSTEMS) or clone 4B12 (NEOMARKERS; LAB VISION). Images are captured to quantify the percentage of tissue area occupied by CD4 by using PHOTOSHOP CS2, version 9.0 imaging software (ADOBE SYSTEMS) with plug-ins from REINDEER GRAPHICS.

To test for differences in cell count between treated versus untreated patients, the two-sample t test with equal variance can be used. Hotteling's T² test can be used to test for changes in both compartments investigated simultaneously. Because these methods rely on normality assumptions, standard diagnostics are employed to assess the normality assumption.

In further embodiments, practice of the methods of the present disclosure protect the CD4 T cell population in lymphoid aggregates of the rectum.

### Anti-Fibrotic Agents

Specific anti-fibrotic agents contemplated include pirfenidone and compounds of formula (I), (II), (III), (IV), and (V)

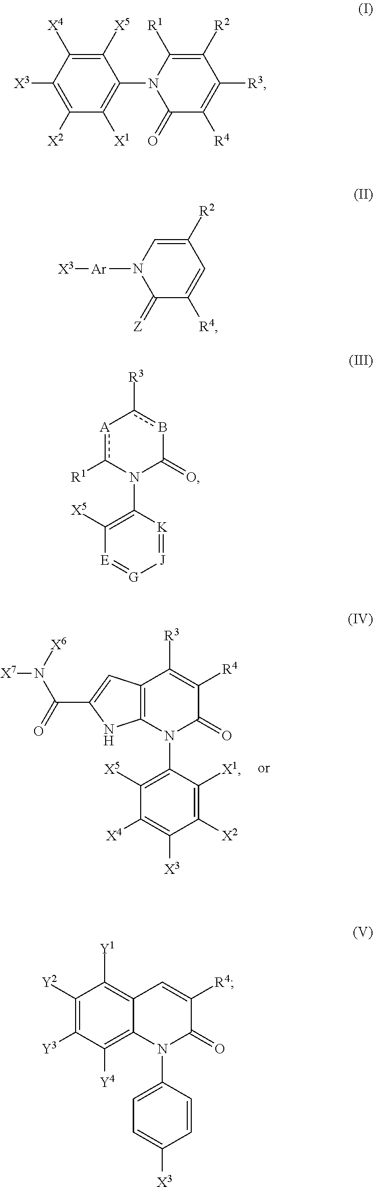

wherein

R¹, R², R³, R⁴, X¹, X², X³, X⁴, X⁵, Y¹, Y², Y³ and Y⁴ are independently selected from the group consisting of H, deuterium, C₁-C₁₀ alkyl, C₁-C₁₀ deuterated alkyl, substituted C₁-C₁₀ alkyl, C₁-C₁₀ alkenyl, substituted C₁-C₁₀ alkenyl, C₁-C₁₀ thioalkyl, C₁-C₁₀ alkoxy, substituted alkoxy, cycloalkyl, substituted cycloalkyl, heterocycloalkyl, substituted heterocycloalkyl, heteroalkyl, substituted heteroalkyl, aryl, substituted aryl, heteroaryl, substituted heteroaryl, halogen, hydroxyl, C₁-C₁₀ alkoxyalkyl, C₁-C₁₀ carboxy, C₁-C₁₀ alkoxycarbonyl, CO-uronide, CO-monosaccharide, CO-oligosaccharide, and CO-polysaccharide;

X⁶ and X⁷ are independently selected from the group consisting of hydrogen, aryl, substituted aryl, heteroaryl, substituted heteroaryl, cycloalkyl, substituted cycloalkyl, heterocycloalkyl, substituted heterocycloalkyl, alkylenylaryl, alkylenylheteroaryl, alkylenylheterocycloalkyl, alkylenylcycloalkyl, or X⁶ and X⁷ together form an optionally substituted 5 or 6 membered heterocyclic ring; and

Ar is pyridinyl or phenyl; and Z is O or S;

or a pharmaceutically acceptable salt, ester, solvate, or prodrug of pirfenidone or the compound of formula (I), (II), (III), (IV), or (V).

In some embodiments, R¹, R², R³, R⁴, X¹, X², X³, X⁴, X⁵, Y¹, Y², Y³, and Y⁴ are independently optionally substituted pyrazinyl, optionally substituted pyridazinyl, optionally substituted pyrrolyl, optionally substituted thiophenyl, optionally substituted thiazolyl, optionally substituted oxazolyl, optionally substituted imidazolyl, optionally substituted isoxazolyl, optionally substituted pyrazolyl, optionally substituted isothiazolyl, optionally substituted napthyl, optionally substituted quinolinyl, optionally substituted isoquinolinyl, optionally substituted quinoxalinyl, optionally substituted benzothiazolyl, optionally substituted benzothiophenyl, optionally substituted benzofuranyl, optionally substituted indolyl, or optionally substituted benzimidazolyl.

The anti-fibrotic therapeutic can be a compound of formula (II), wherein X³ is H, OH, or C₁₋₁₀alkoxy, Z is O, and R² is methyl, C(═O)H, C(═O)CH₃, C(═O)O-glucosyl, fluoromethyl, difluoromethyl, trifluoromethyl, methylmethoxyl, methylhydroxyl, or phenyl; and R⁴ is H or hydroxyl.

Some specific contemplated compounds of formula (II) include

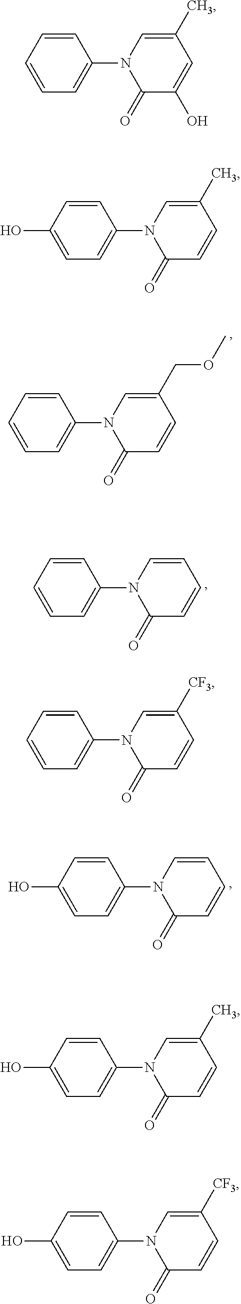

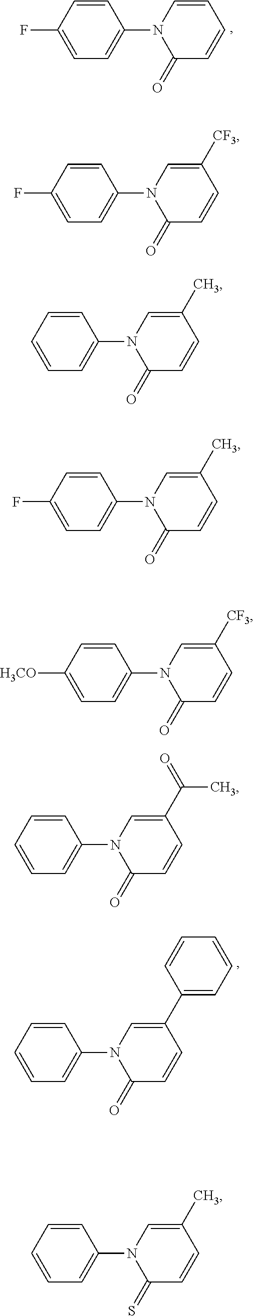

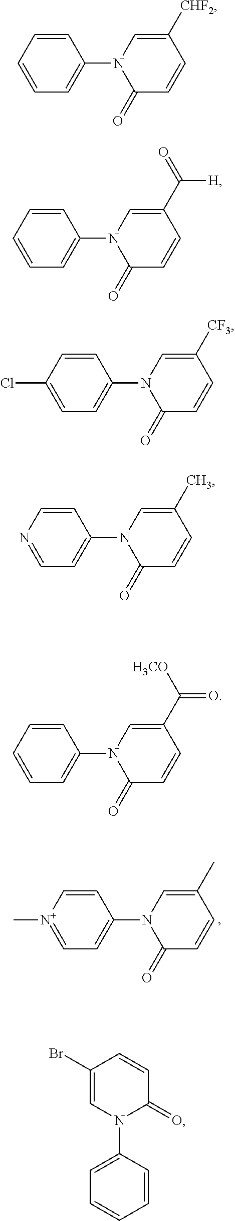

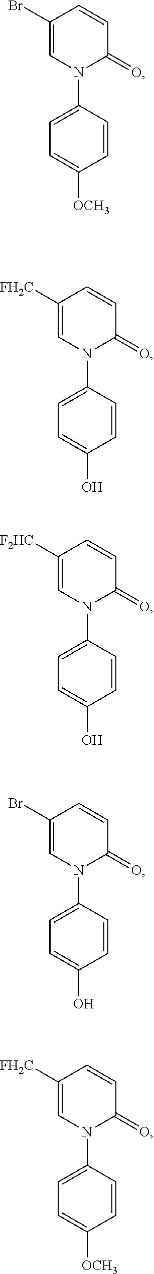

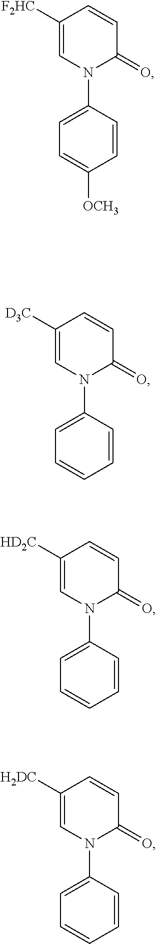

a compound listed in Table 1, below, and pharmaceutically acceptable salts, esters, solvates, and prodrugs thereof.

The term “alkyl” used herein refers to a saturated or unsaturated straight or branched chain hydrocarbon group of one to ten carbon atoms, including, but not limited to, methyl, ethyl, n-propyl, isopropyl, n-butyl, isobutyl, tert-butyl, n-hexyl, and the like. Alkyls of one to six carbon atoms are also contemplated. The term “alkyl” includes “bridged alkyl,” i.e., a bicyclic or polycyclic hydrocarbon group, for example, norbornyl, adamantyl, bicyclo[2.2.2]octyl, bicyclo[2.2.1]heptyl, bicyclo[3.2.1]octyl, or decahydronaphthyl. Alkyl groups optionally can be substituted, for example, with hydroxy (OH), halo, aryl, heteroaryl, cycloalkyl, heterocycloalkyl, and amino. It is specifically contemplated that in the derivatives and analogs described herein the alkyl group consists of 1-40 carbon atoms, preferably 1-25 carbon atoms, preferably 1-15 carbon atoms, preferably 1-12 carbon atoms, preferably 1-10 carbon atoms, preferably 1-8 carbon atoms, and preferably 1-6 carbon atoms. “Heteroalkyl” is defined similarly as alkyl, except the heteroalkyl contains at least one heteroatom independently selected from the group consisting of oxygen, nitrogen, and sulfur.

As used herein, the term “cycloalkyl” refers to a cyclic hydrocarbon group, e.g., cyclopropyl, cyclobutyl, cyclohexyl, and cyclopentyl. “Heterocycloalkyl” is defined similarly as cycloalkyl, except the ring contains one to three heteroatoms independently selected from the group consisting of oxygen, nitrogen, and sulfur. Nonlimiting examples of heterocycloalkyl groups include piperidine, tetrahydrofuran, tetrahydropyran, dihydrofuran, morpholine, thiophene, and the like. Cycloalkyl and heterocycloalkyl groups can be saturated or partially unsaturated ring systems optionally substituted with, for example, one to three groups, independently selected from the group consisting of alkyl, alkyleneOH, C(O)NH₂, NH₂, oxo (═O), aryl, haloalkyl, halo, and OH. Heterocycloalkyl groups optionally can be further N-substituted with alkyl, hydroxyalkyl, alkylenearyl, or alkyleneheteroaryl.

The term “alkenyl” used herein refers to a straight or branched chain hydrocarbon group of two to ten carbon atoms containing at least one carbon double bond including, but not limited to, 1-propenyl, 2-propenyl, 2-methyl-1-propenyl, 1-butenyl, 2-butenyl, and the like.

The term “halo” used herein refers to fluoro, chloro, bromo, or iodo.

The term “alkylene” used herein refers to an alkyl group having a substituent. For example, the term “alkylene aryl” refers to an alkyl group substituted with an aryl group. The alkylene group is optionally substituted with one or more substituent previously listed as an optional alkyl substituent. For example, an alkylene group can be —CH₂CH₂—.

As used herein, the term “alkenylene” is defined identical as “alkylene,” except the group contains at least one carbon-carbon double bond.

As used herein, the term “aryl” refers to a monocyclic or polycyclic aromatic group, preferably a monocyclic or bicyclic aromatic group, e.g., phenyl or naphthyl. Unless otherwise indicated, an aryl group can be unsubstituted or substituted with one or more, and in particular one to four groups independently selected from, for example, halo, alkyl, alkenyl, OCF₃, NO₂, CN, NC, OH, alkoxy, amino, CO₂H, CO₂alkyl, aryl, and heteroaryl. Exemplary aryl groups include, but are not limited to, phenyl, naphthyl, tetrahydronaphthyl, chlorophenyl, methylphenyl, methoxyphenyl, trifluoromethylphenyl, nitrophenyl, 2,4-methoxychlorophenyl, and the like.

As used herein, the term “heteroaryl” refers to a monocyclic or bicyclic ring system containing one or two aromatic rings and containing at least one nitrogen, oxygen, or sulfur atom in an aromatic ring. Unless otherwise indicated, a heteroaryl group can be unsubstituted or substituted with one or more, and in particular one to four, substituents selected from, for example, halo, alkyl, alkenyl, OCF₃, NO₂, CN, NC, OH, alkoxy, amino, CO₂H, CO₂alkyl, aryl, and heteroaryl. Examples of heteroaryl groups include, but are not limited to, thienyl, furyl, pyridyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl.

The term “deuterated alkyl” used herein refers to an alkyl group substituted with one or more deuterium atoms (D).

The term “thioalkyl” used herein refers to one or more thio groups appended to an alkyl group.

The term “hydroxyalkyl” used herein refers to one or more hydroxy groups appended to an alkyl group.

The term “alkoxy” used herein refers to straight or branched chain alkyl group covalently bonded to the parent molecule through an —O— linkage. Examples of alkoxy groups include, but are not limited to, methoxy, ethoxy, propoxy, isopropoxy, butoxy, n-butoxy, sec-butoxy, t-butoxy and the like.

The term “alkoxyalkyl” used herein refers to one or more alkoxy groups appended to an alkyl group.

The term “arylalkoxy” used herein refers to a group having an aryl appended to an alkoxy group. A non-limiting example of an arylalkoxy group is a benzyloxy (Ph-CH₂—O—).

The term “amino” as used herein refers to —NR₂, where R is independently hydrogen or alkyl. Non-limiting examples of amino groups include NH₂ and N(CH₃)₂.

The term “amido” as used herein refers to —NHC(O)alkyl or —NHC(O)H. A non-limiting example of an amido group is —NHC(O)CH₃.

The term “carboxy” or “carboxyl” used herein refers to —COOH or its deprotonated form —COO⁻. C₁₋₁₀carboxy refers to optionally substituted alkyl or alkenyl groups having a carboxy moiety. Examples include, but are not limited to, —CH₂COOH, —CH₂CH(COOH)CH₃, and —CH₂CH₂CH₂COOH.

The term “alkoxycarbonyl” refers to —(CO)—O-alkyl, wherein the alkyl group can optionally be substituted. Examples of alkoxycarbonyl groups include, but are not limited to, methoxycarbonyl group, ethoxycarbonyl group, propoxycarbonyl group, and the like.

The term “alkylcarbonyl” refers to —(CO)-alkyl, wherein the alkyl group can optionally be substituted. Examples of alkylcarbonyl groups include, but are not limited to, methylcarbonyl group, ethylcarbonyl group, propylcarbonyl group, and the like.

The term “sulfonamido” refers to —SO₂NR₂ where R is independently hydrogen or an optionally substituted alkyl group. Examples of a sulfonamido group include, but are not limited to, —SO₂N(CH₃)₂ and —SO₂NH₂.

The term “sulfonyl” refers to —SO2alkyl, wherein the alkyl group can optionally be substituted. One example of a sulfonyl group is methylsulfonyl (e.g., —SO2CH3).

Carbohydrates are polyhydroxy aldehydes or ketones, or substances that yield such compounds upon hydrolysis. Carbohydrates comprise the elements carbon (C), hydrogen (H) and oxygen (O) with a ratio of hydrogen twice that of carbon and oxygen. In their basic form, carbohydrates are simple sugars or monosaccharides. These simple sugars can combine with each other to form more complex carbohydrates. The combination of two simple sugars is a disaccharide. Carbohydrates consisting of two to ten simple sugars are called oligosaccharides, and those with a larger number are called polysaccharides.

The term “uronide” refers to a monosaccharide having a carboxyl group on the carbon that is not part of the ring. The uronide name retains the root of the monosaccharide, but the -ose sugar suffix is changed to -uronide. For example, the structure of glucuronide corresponds to glucose.

As used herein, a radical indicates species with a single, unpaired electron such that the species containing the radical can be covalently bonded to another species. Hence, in this context, a radical is not necessarily a free radical. Rather, a radical indicates a specific portion of a larger molecule. The term “radical” can be used interchangeably with the term “group.”

As used herein, a substituted group is derived from the unsubstituted parent structure in which there has been an exchange of one or more hydrogen atoms for another atom or group. A “substituent group,” as used herein, means a group selected from the following moieties:

(A) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, unsubstituted alkoxy, unsubstituted aryloxy, trihalomethanesulfonyl, trifluoromethyl, and

(B) alkyl, heteroalkyl, cycloalkyl, heterocycloalkyl, aryl, amino, amido, carbonyl, thiocarbonyl, alkoxycarbonyl, silyl, sulfonyl, sulfoxyl, alkoxy, aryloxy, and heteroaryl, substituted with at least one substituent selected from:
(i) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, unsubstituted alkoxy, unsubstituted aryloxy, trihalomethanesulfonyl, trifluoromethyl, and
(ii) alkyl, heteroalkyl, cycloalkyl, heterocycloalkyl, aryl, amino, amido, carbonyl, thiocarbonyl, alkoxycarbonyl, silyl, sulfonyl, sulfoxyl, alkoxy, aryloxy, and heteroaryl, substituted with at least one substituent selected from:
   (a) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, unsubstituted alkoxy, unsubstituted aryloxy, trihalomethanesulfonyl, trifluoromethyl, and
   (b) alkyl, heteroalkyl, cycloalkyl, heterocycloalkyl, aryl, amino, amido, carbonyl, thiocarbonyl, alkoxycarbonyl, silyl, sulfonyl, sulfoxyl, alkoxy, aryloxy, and heteroaryl, substituted with at least one substituent selected from —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, unsubstituted alkoxy, unsubstituted aryloxy, trihalomethanesulfonyl, trifluoromethyl.

In some embodiments, the substituent group is a “size-limited substituent” or “size-limited substituent group,” which refers to a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

In some embodiments, the substituent group is a “lower substituent” or “lower substituent group,” which refers to a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

In some cases, the substituent group(s) is (are) one or more group(s) individually and independently selected from alkyl, cycloalkyl, aryl, fused aryl, heterocyclyl, heteroaryl, hydroxy, alkoxy, aryloxy, mercapto, alkylthio, arylthio, cyano, halo, carbonyl, thiocarbonyl, alkoxycarbonyl, nitro, silyl, trihalomethanesulfonyl, trifluoromethyl, and amino, including mono- and di-substituted amino groups, and the protected derivatives thereof.

The protecting groups that can form the protective derivatives of the above substituents are known to those of skill in the art and can be found in references such as Greene and Wuts, Protective Groups in Organic Synthesis; 3^{rd} Edition, John Wiley and Sons: New York, 2006. Wherever a substituent is described as “optionally substituted” that substituent can be substituted with the above-described substituents.

Asymmetric carbon atoms can be present. All such isomers, including diastereomers and enantiomers, as well as the mixtures thereof, are intended to be included in the scope of the disclosure herein. In certain cases, compounds can exist in tautomeric forms. All tautomeric forms are intended to be included in the scope of the disclosure herein. Likewise, when compounds contain an alkenyl or alkenylene group, there exists the possibility of cis- and trans-isomeric forms of the compounds. Both cis- and trans-isomers, as well as the mixtures of cis- and trans-isomers, are contemplated.

Anti-fibrotic compounds that can be used in the disclosed methods also include those described in U.S. Patent Publication No. 2007/0049624 (US national stage of WO 05/0047256), in International Publication Nos. WO 03/068230, WO 08/003,141, or WO 08/157,786, or in U.S. Pat. Nos. 5,962,478; 6,300,349; 6,090,822; 6,114,353; Re. 40,155; 6,956,044; or 5,310,562. Synthesis of the compounds used in the disclosed methods can be by any means known in the art, including those described in the patents and patent publications listed herein. Other synthetic means can be used and are within the knowledge of the skilled artisan.

Compounds that can be used in the disclosed methods include those described in U.S. Patent Publication No. 2007/0049624 (US national stage of WO 05/0047256), International Publication No. WO 03/068230, WO 08/003,141, WO 08/157,786, or in U.S. Pat. Nos. 5,962,478; 6,300,349; 6,090,822; 6,114,353; Re. 40,155; 6,956,044; or 5,310,562. Synthesis of the compounds used in the disclosed methods can be by any means known in the art, including those described in the patents and patent publications listed herein. Other synthetic means can be used and are within the knowledge of the skilled artisan.

One class of anti-fibrotic compounds contemplated for use in the disclosed methods is a deuterated (D) form of any of the compounds disclosed herein. One specific such compound is a compound having a CD₃ moiety and/or a D to replace any or all of the methyl or hydrogens of the compound, such as pirfenidone. Examples include

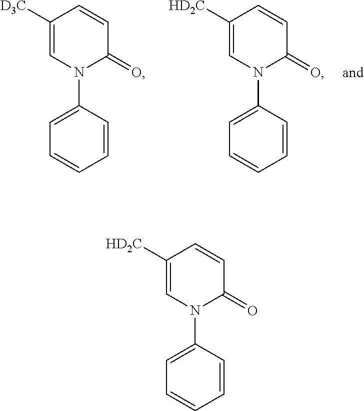

Descriptions of methods of synthesis of these compounds can be found in International Patent Publication No. WO 08/157,786.

Some specific compounds of formula (I), (II), (III), or (IV) are listed in Table 1. Description of the synthesis of these compounds can be found in WO 09/149,188, the disclosure of which is incorporated by reference herein.

TABLE 1

| Cmpd | |
|---|---|
| No. | Structure |
| 1 | 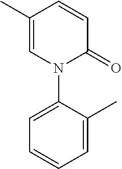 |
| 2 | 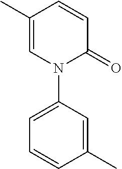 |
| 3 | 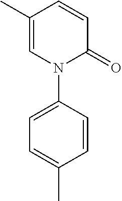 |
| 4 | 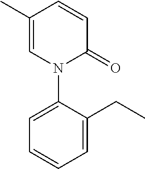 |
| 5 | 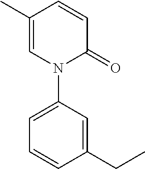 |
| 6 | 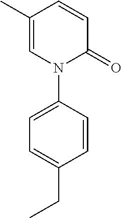 |
| 7 | 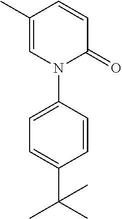 |
| 8 | 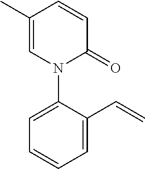 |
| 9 | 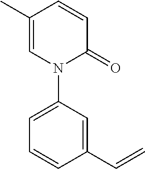 |
| 10 | 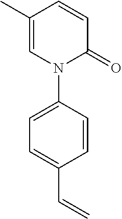 |
| 11 | 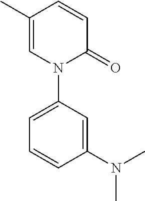 |
| 12 | 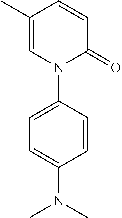 |
| 13 | 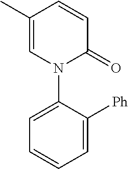 |
| 14 | 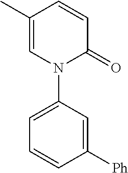 |
| 15 | 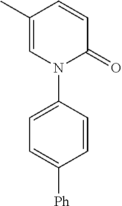 |
| 16 | 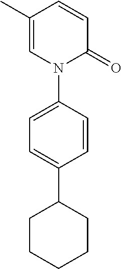 |
| 17 | 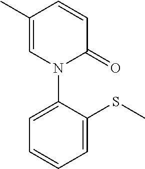 |
| 18 | 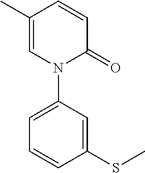 |
| 19 | 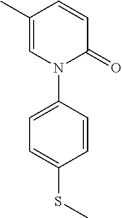 |
| 20 | 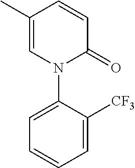 |
| 21 | 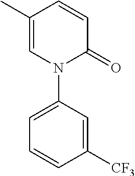 |
| 22 | 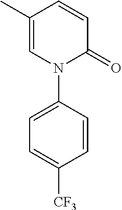 |
| 23 | 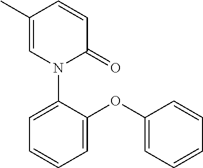 |
| 24 | 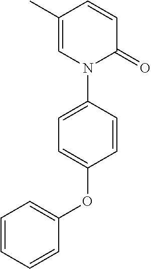 |
| 25 | 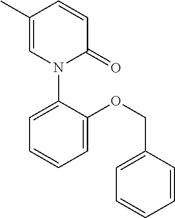 |
| 26 | 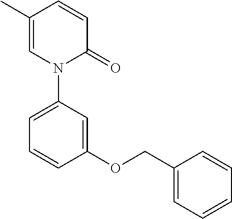 |
| 27 | 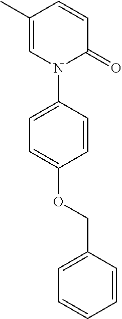 |
| 28 | 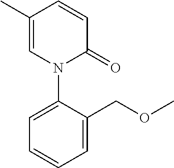 |
| 29 | 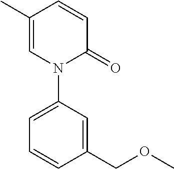 |
| 30 | 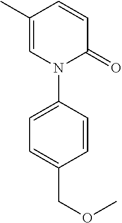 |
| 31 | 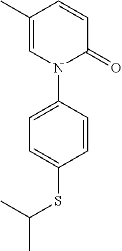 |
| 32 | 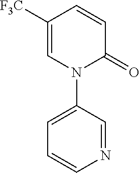 |
| 33 | 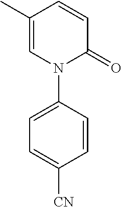 |
| 34 | 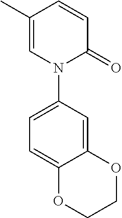 |
| 35 | 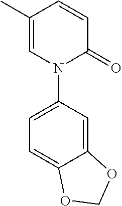 |
| 36 | 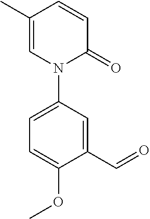 |
| 37 | 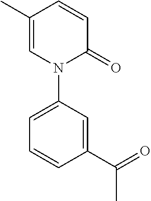 |
| 38 | 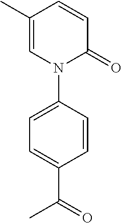 |
| 39 | 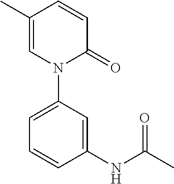 |
| 40 | 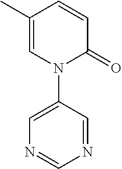 |
| 41 | 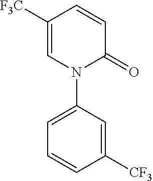 |
| 42 | 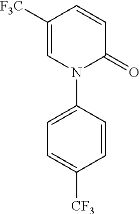 |
| 43 | 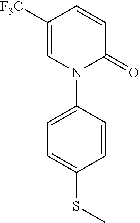 |
| 44 | 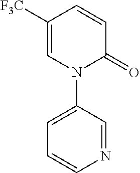 |
| 45 | 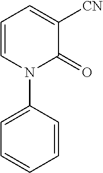 |
| 46 | 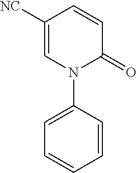 |
| 47 | 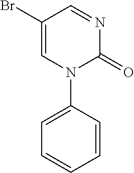 |
| 48 | 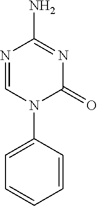 |
| 49 | 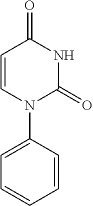 |
| 50 | 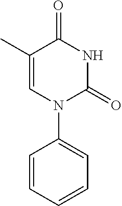 |
| 51 | 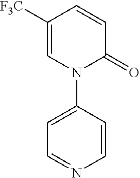 |
| 52 | 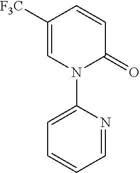 |
| 53 | 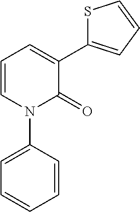 |
| 54 | 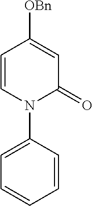 |
| 55 | 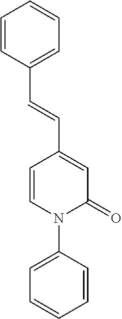 |
| 56 | 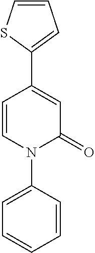 |
| 57 | 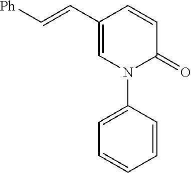 |
| 58 | 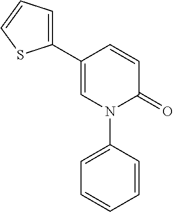 |
| 59 | 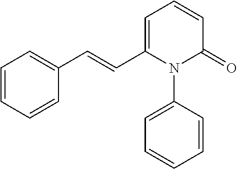 |
| 60 | 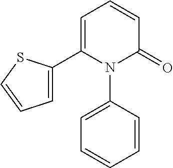 |
| 61 | 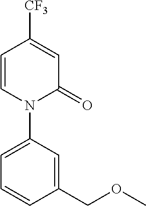 |
| 62 | 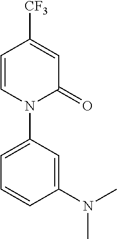 |
| 63 | 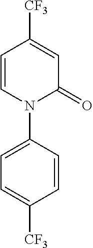 |
| 64 | 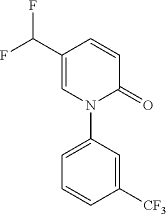 |
| 65 | 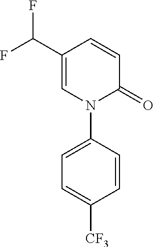 |
| 66 | 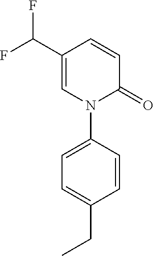 |
| 67 | 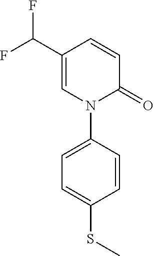 |
| 68 | 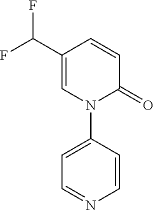 |
| 69 | 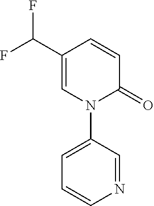 |
| 70 | 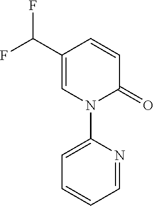 |
| 71 | 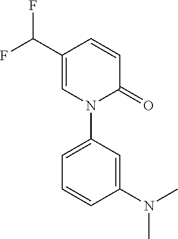 |
| 72 | 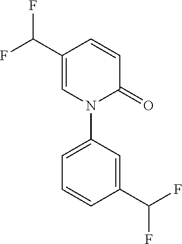 |
| 73 | 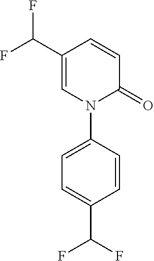 |
| 74 | 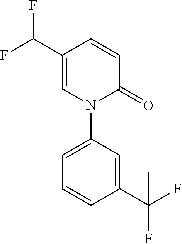 |
| 75 | 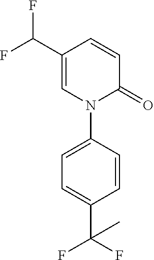 |
| 76 | 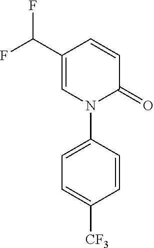 |
| 77 | 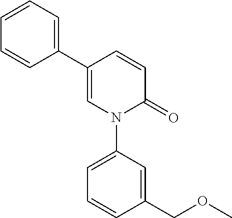 |
| 78 | 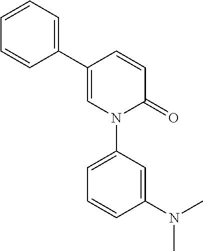 |
| 79 | 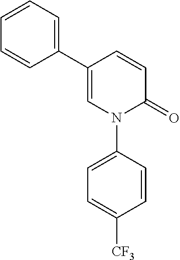 |
| 80 | 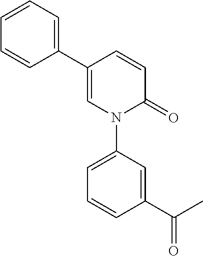 |
| 81 | 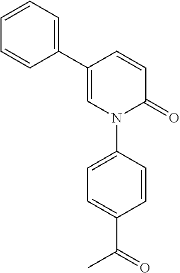 |
| 82 | 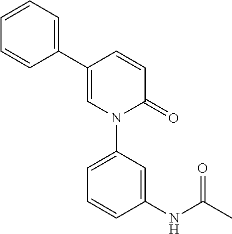 |
| 83 | 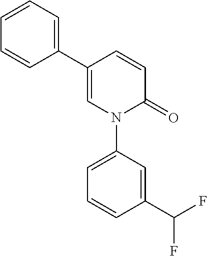 |
| 84 | 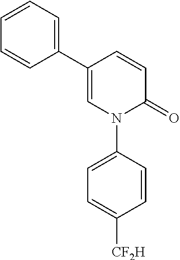 |
| 85 | 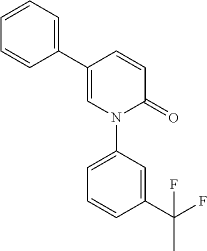 |
| 86 | 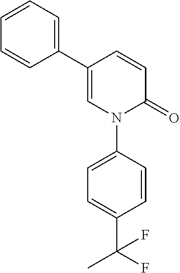 |
| 87 | 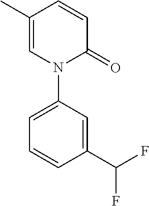 |
| 88 | 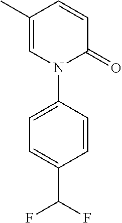 |
| 89 | 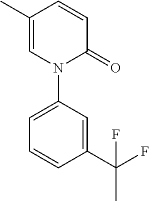 |
| 90 | 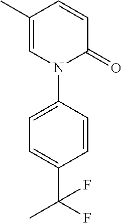 |
| 91 | 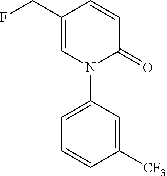 |
| 92 | 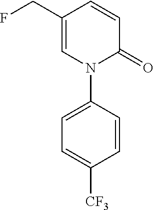 |
| 93 | 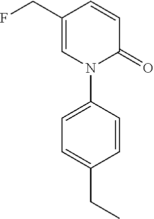 |
| 94 | 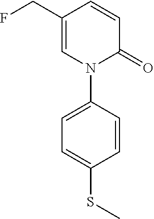 |
| 95 | 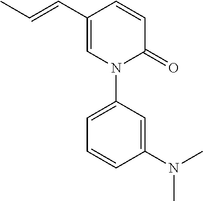 |
| 96 | 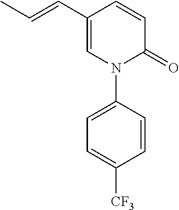 |
| 97 | 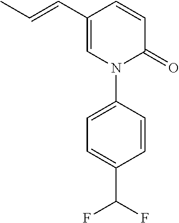 |
| 98 | 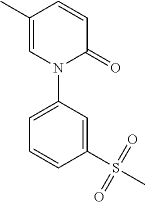 |
| 99 | 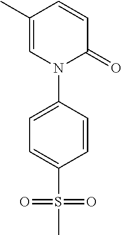 |
| 100 | 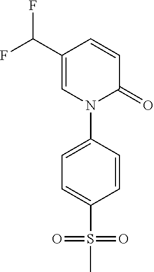 |
| 101 | 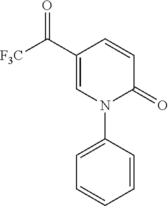 |
| 102 | 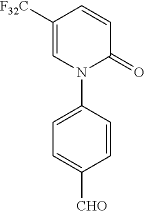 |
| 103 | 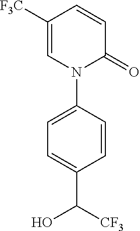 |
| 104 | 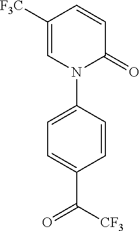 |
| 105 | 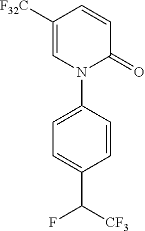 |
| 106 | 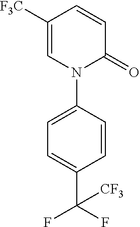 |
| 107 | 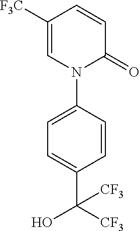 |
| 108 | 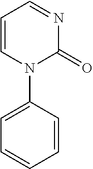 |
| 109 | 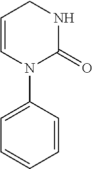 |
| 110 | 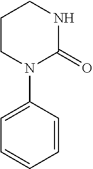 |
| 111 | 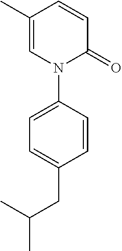 |
| 112 | 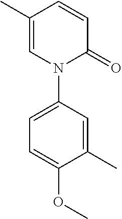 |
| 113 | 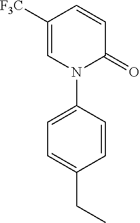 |
| 114 | 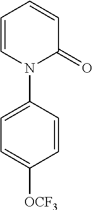 |
| 115 | 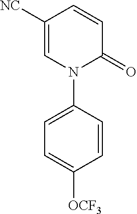 |
| 116 | 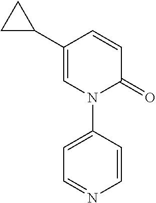 |
| 117 | 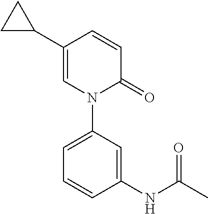 |
| 118 | 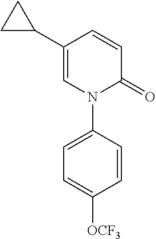 |
| 119 | 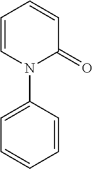 |
| 120 | 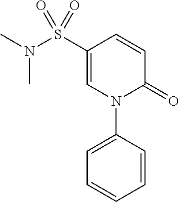 |
| 121 | 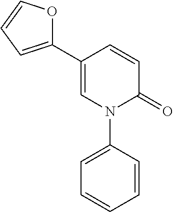 |
| 122 | 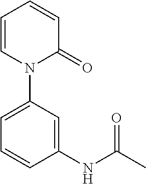 |
| 123 | 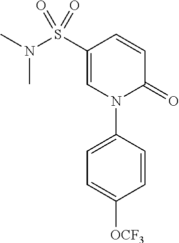 |
| 124 | 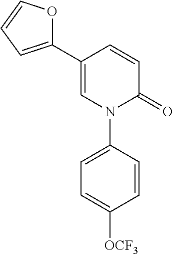 |
| 125 | 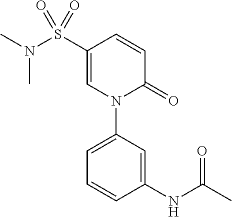 |
| 126 | 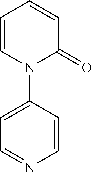 |
| 127 | 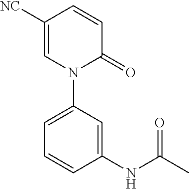 |
| 128 | 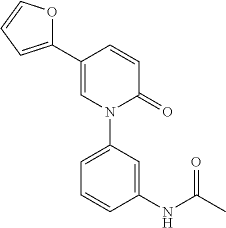 |
| 129 | 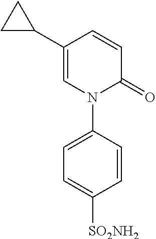 |
| 130 | 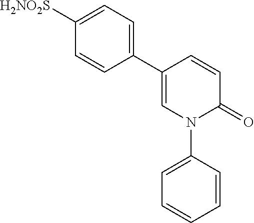 |
| 131 | 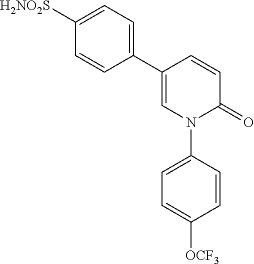 |
| 132 | 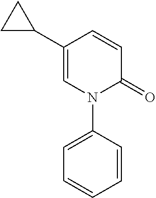 |
| 133 | 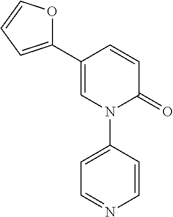 |
| 134 | 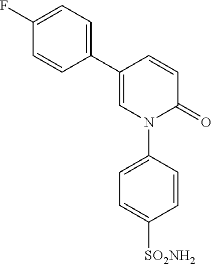 |
| 135 | 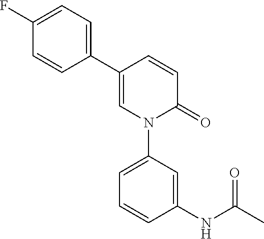 |
| 136 | 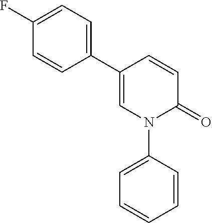 |
| 137 | 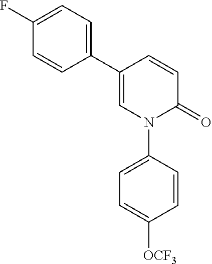 |
| 138 | 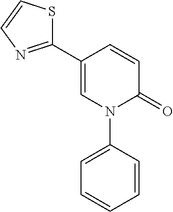 |
| 139 | 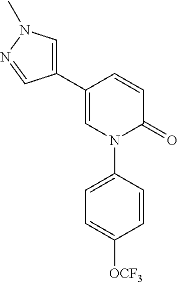 |
| 140 | 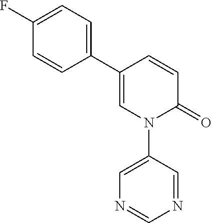 |
| 141 | 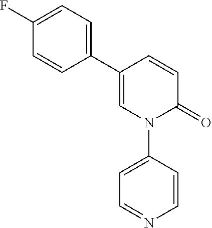 |
| 142 | 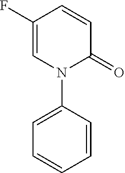 |
| 143 | 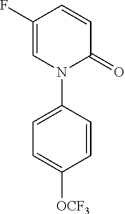 |
| 144 | 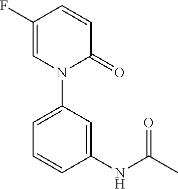 |
| 145 | 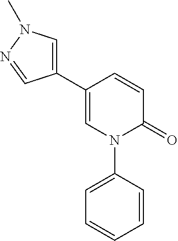 |
| 146 | 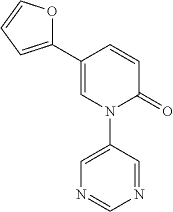 |
| 147 | 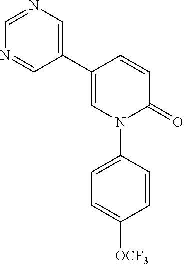 |
| 148 | 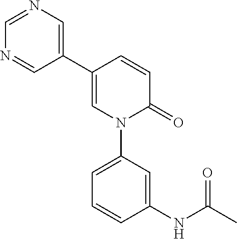 |
| 149 | 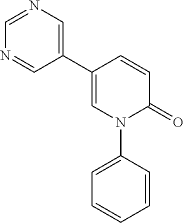 |
| 150 | 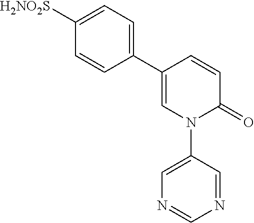 |
| 151 | 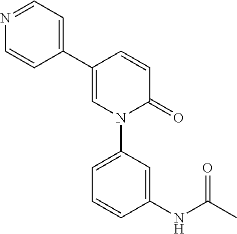 |
| 152 | 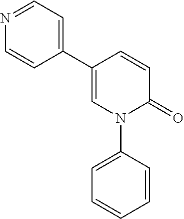 |
| 153 | 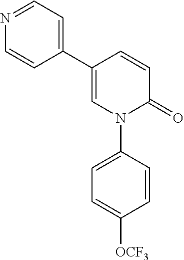 |
| 154 | 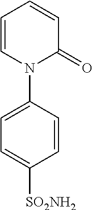 |
| 155 | 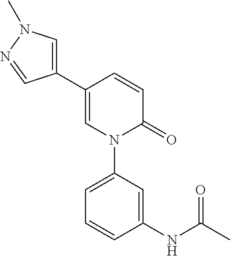 |
| 156 | 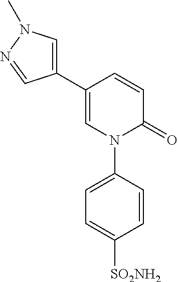 |
| 157 | 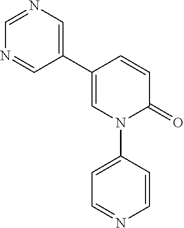 |
| 158 | 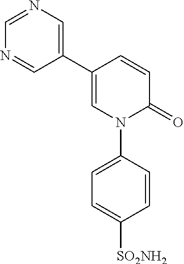 |
| 159 | 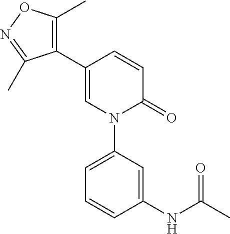 |
| 160 | 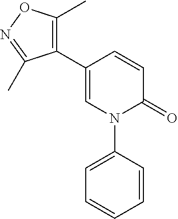 |
| 161 | 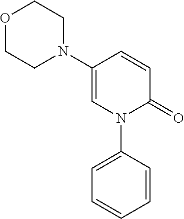 |
| 162 | 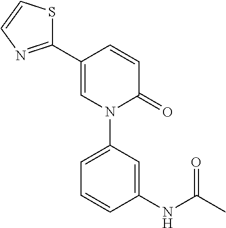 |
| 163 | 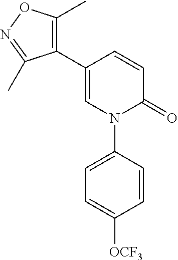 |
| 164 | 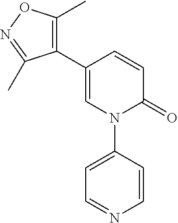 |
| 165 | 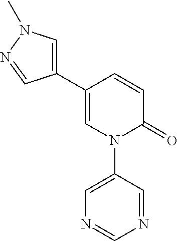 |
| 166 | 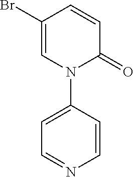 |
| 167 | 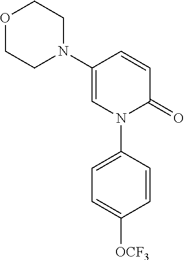 |
| 168 | 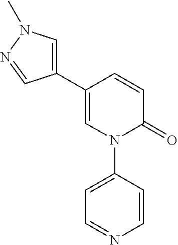 |
| 169 | 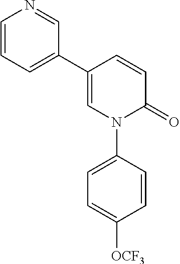 |
| 170 | 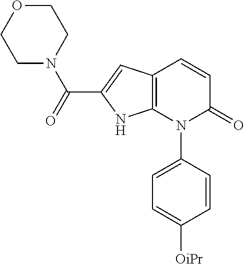 |
| 171 | 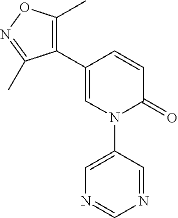 |
| 172 | 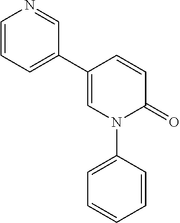 |
| 173 | 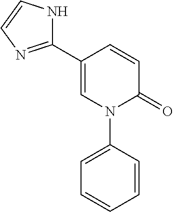 |
| 174 | 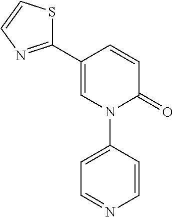 |
| 175 | 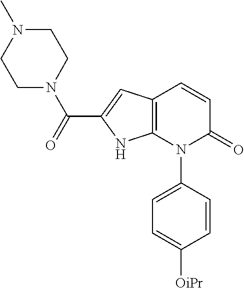 |
| 176 | 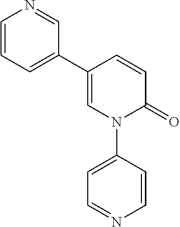 |
| 177 | 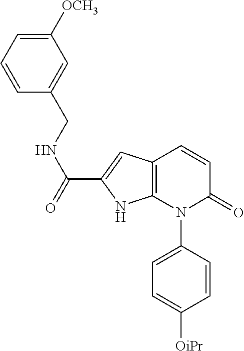 |
| 178 | 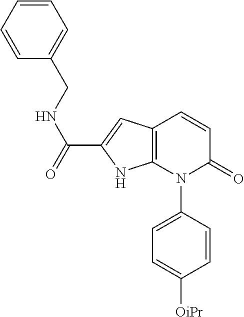 |
| 179 | 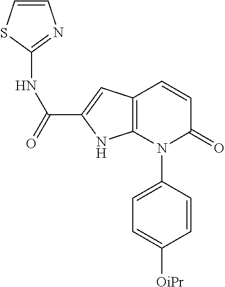 |
| 180 | 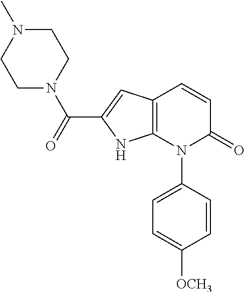 |
| 181 | 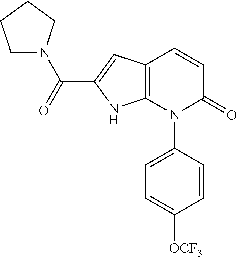 |
| 182 | 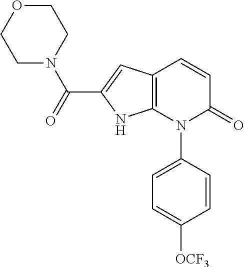 |
| 183 | 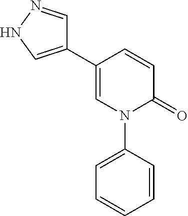 |
| 184 | 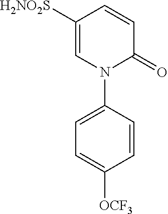 |
| 185 | 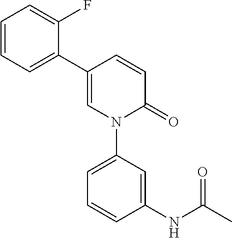 |
| 186 | 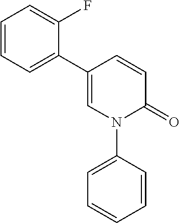 |
| 187 | 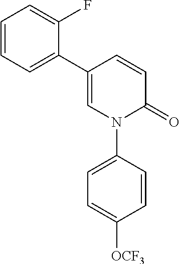 |
| 188 | 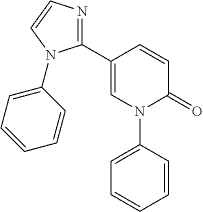 |
| 189 | 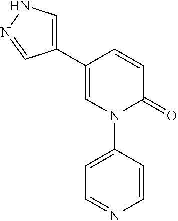 |
| 190 | 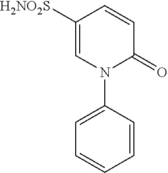 |
| 191 | 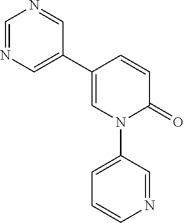 |
| 192 | 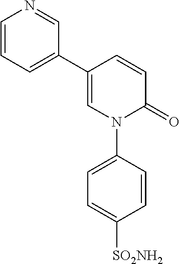 |
| 193 | 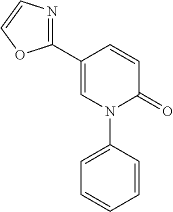 |
| 194 | 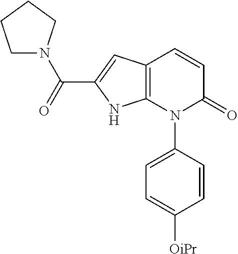 |
| 195 | 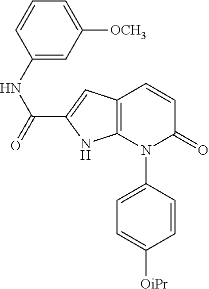 |
| 196 | 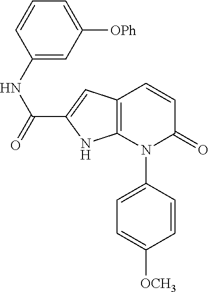 |
| 197 | 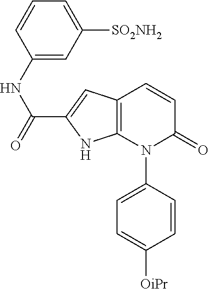 |
| 198 | 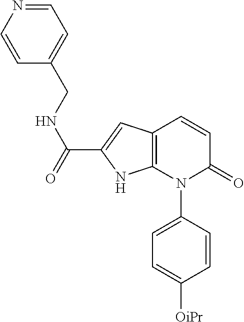 |
| 199 | 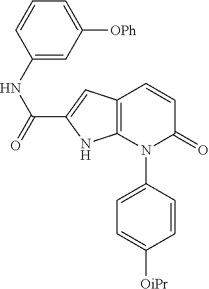 |
| 200 | 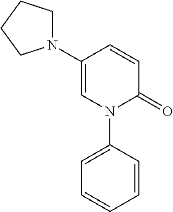 |
| 201 | 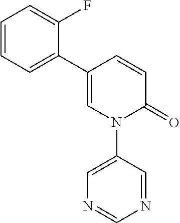 |
| 202 | 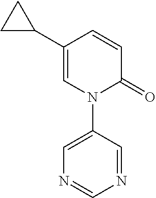 |
| 203 | 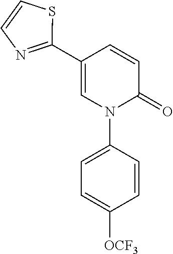 |
| 204 | 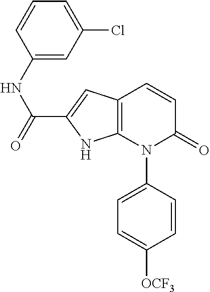 |
| 205 | 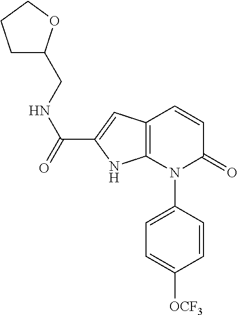 |
| 206 | 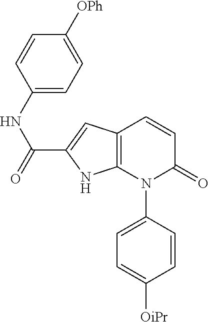 |
| 207 | 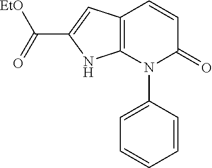 |
| 208 | 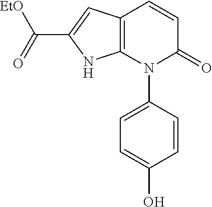 |
| 209 | 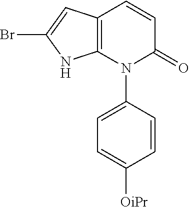 |
| 210 | 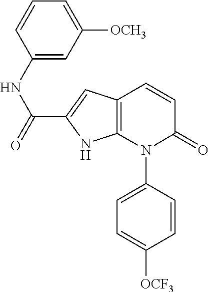 |
| 211 | 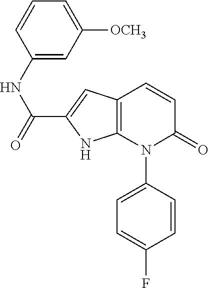 |
| 212 | 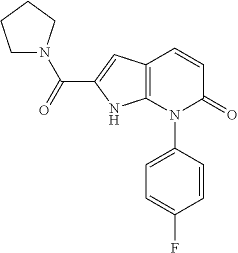 |
| 213 | 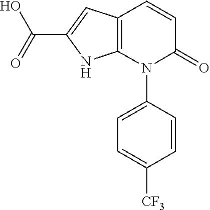 |
| 214 | 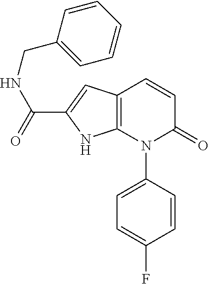 |
| 215 | 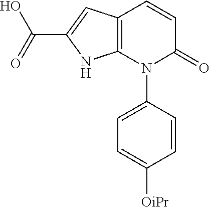 |
| 216 | 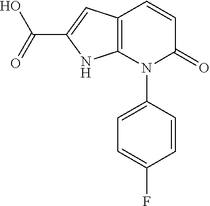 |
| 217 | 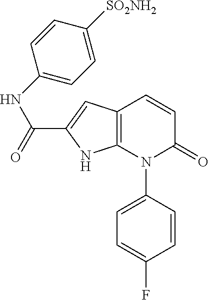 |
| 218 | 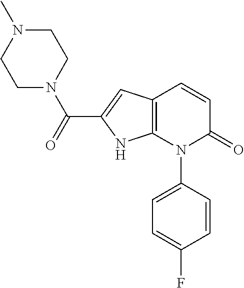 |
| 219 | 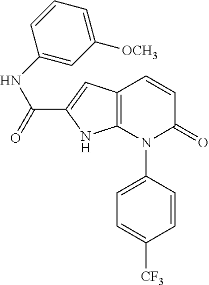 |
| 220 | 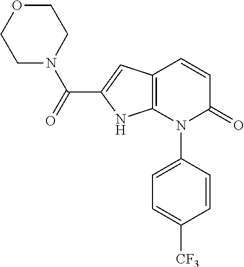 |
| 221 | 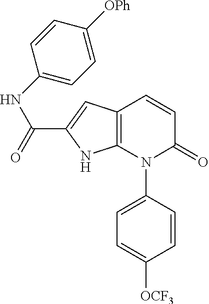 |
| 222 | 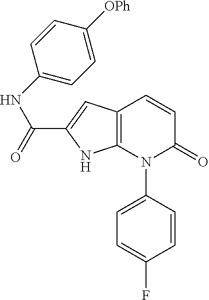 |
| 223 | 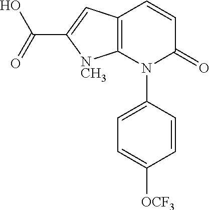 |
| 224 | 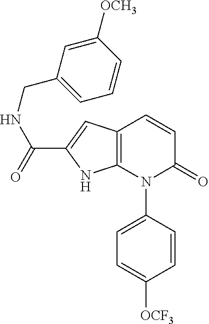 |
| 225 | 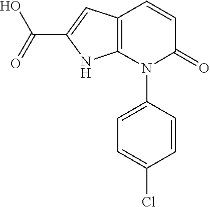 |
| 226 | 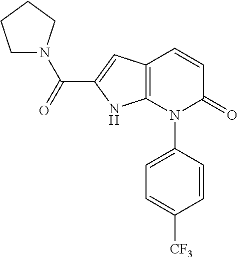 |
| 227 | 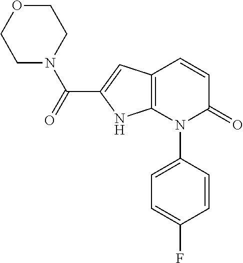 |
| 228 | 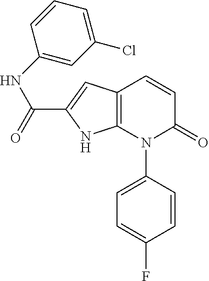 |
| 229 | 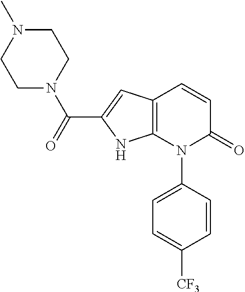 |
| 230 | 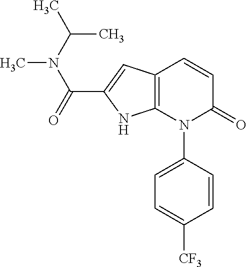 |
| 231 | 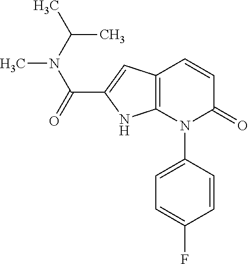 |
| 232 | 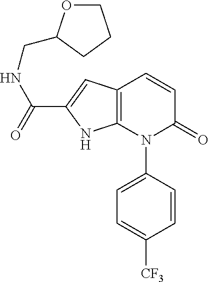 |
| 233 | 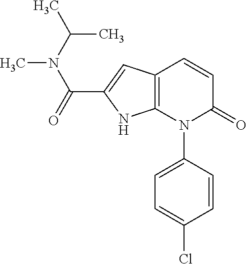 |
| 234 | 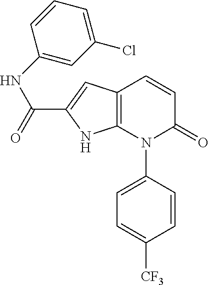 |
| 235 | 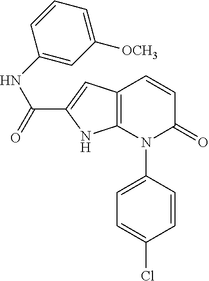 |
| 236 | 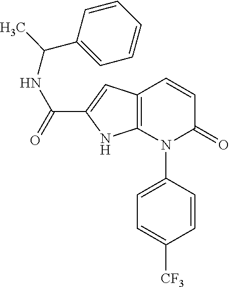 |
| 237 | 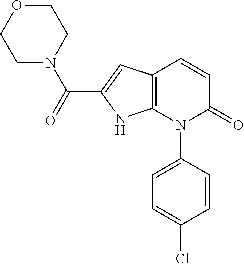 |
| 238 | 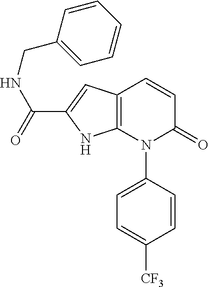 |
| 239 | 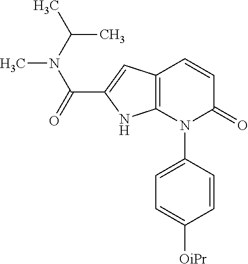 |
| 240 | 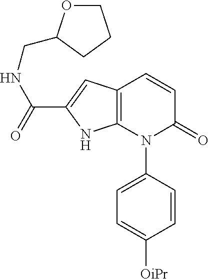 |
| 241 | 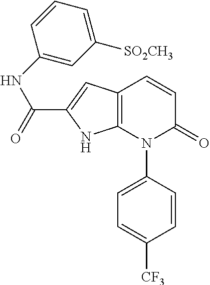 |
| 242 | 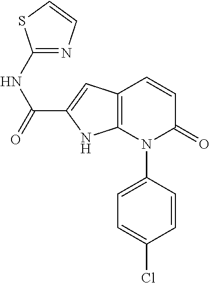 |
| 243 | 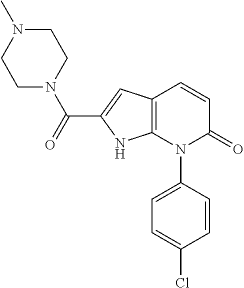 |
| 244 | 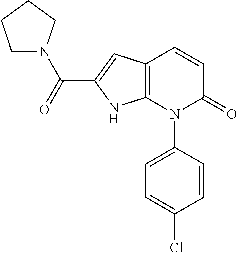 |
| 245 | 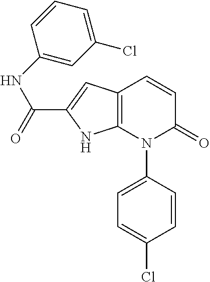 |
| 246 | 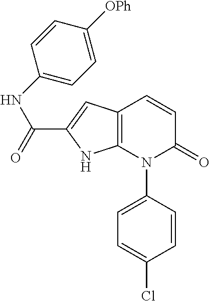 |
| 247 | 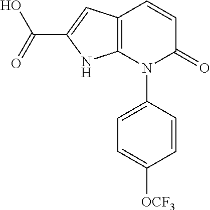 |
| 248 | 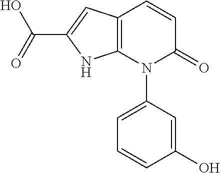 |
| 249 | 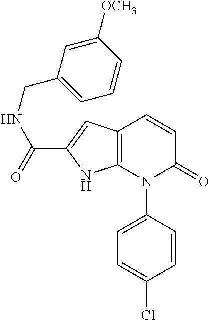 |
| 250 | 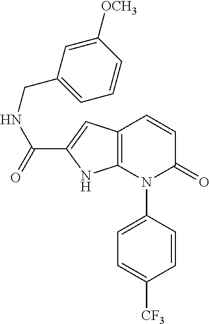 |
| 251 | 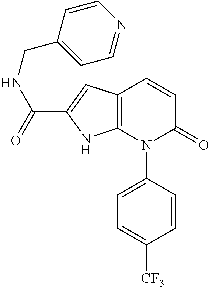 |
| 252 | 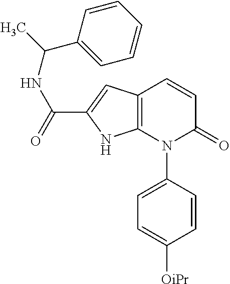 |
| 253 | 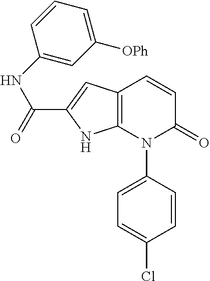 |
| 254 | 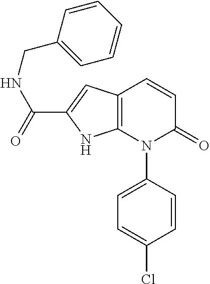 |
| 255 | 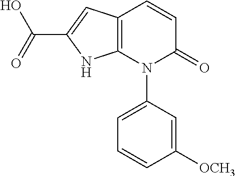 |
| 256 | 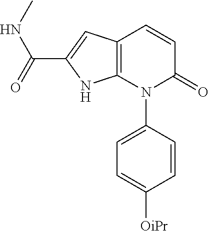 |
| 257 | 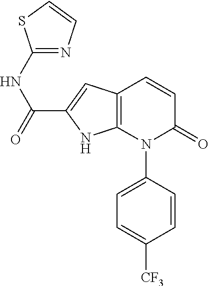 |
| 258 | 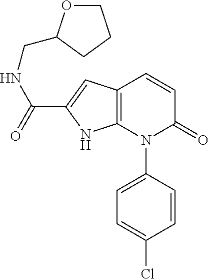 |
| 259 | 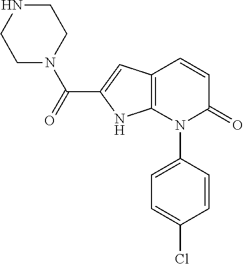 |
| 260 | 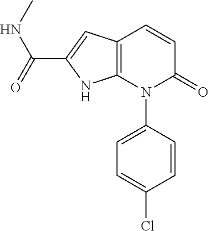 |
| 261 | 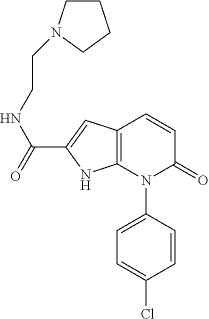 |
| 262 | 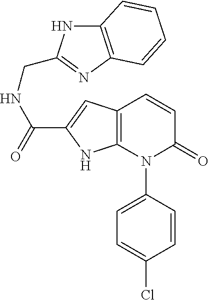 |
| 263 | 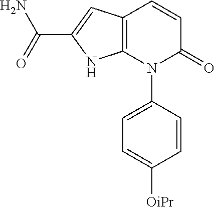 |
| 264 | 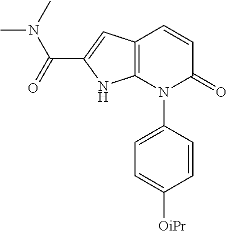 |
| 265 | 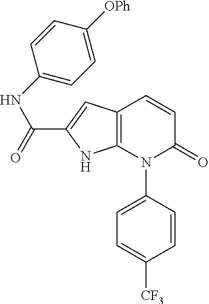 |
| 266 | 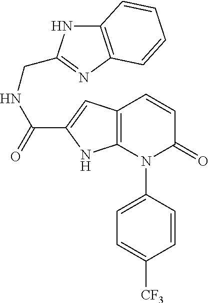 |
| 267 | 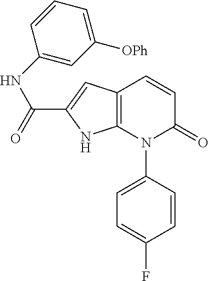 |
| 268 | 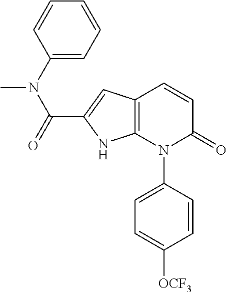 |
| 269 | 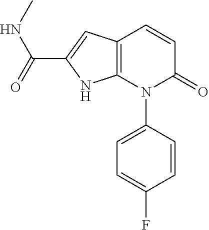 |
| 270 | 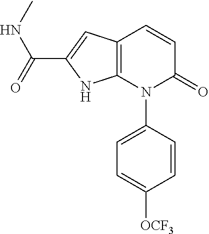 |
| 271 | 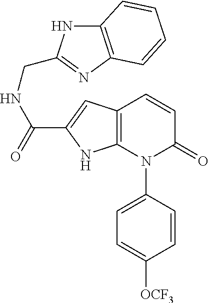 |
| 272 | 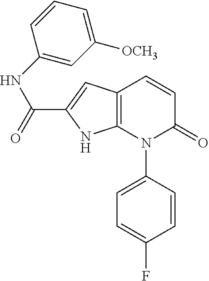 |
| 273 | 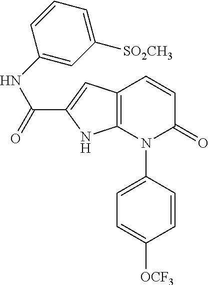 |
| 274 | 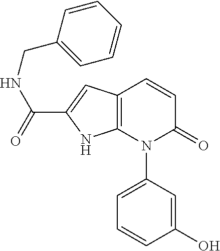 |
| 275 | 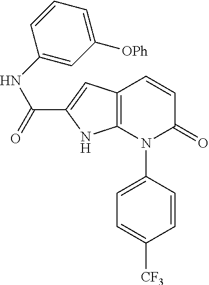 |
| 276 | 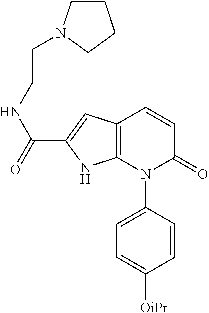 |
| 277 | 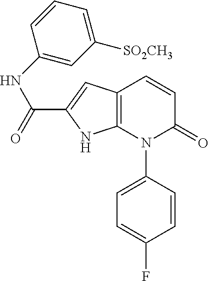 |
| 278 | 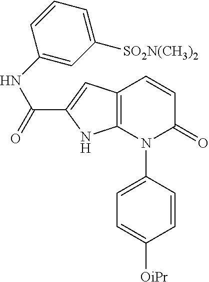 |
| 279 | 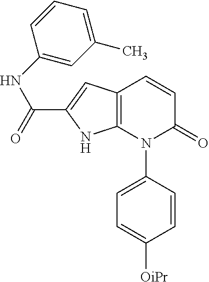 |
| 280 | 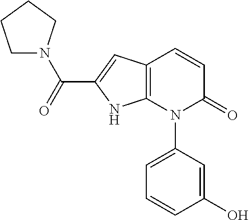 |
| 281 | 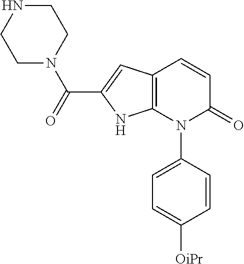 |
| 282 | 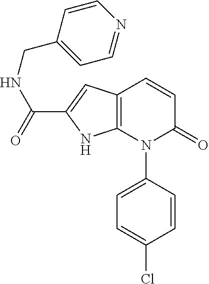 |
| 283 | 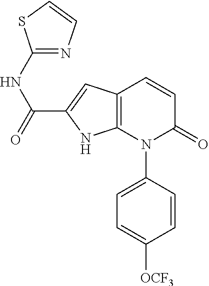 |
| 284 | 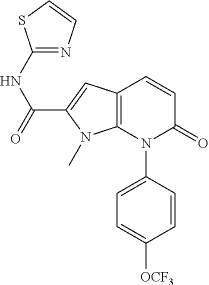 |
| 285 | 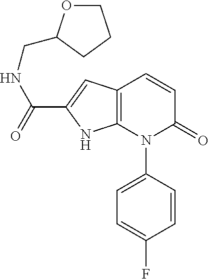 |
| 286 | 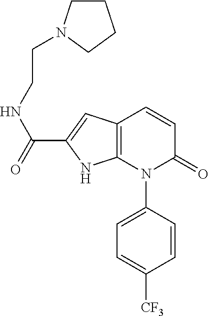 |
| 287 | 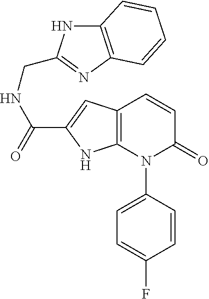 |
| 288 | 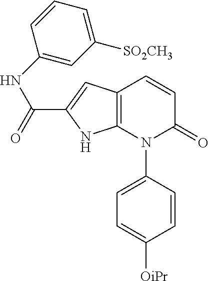 |
| 289 | 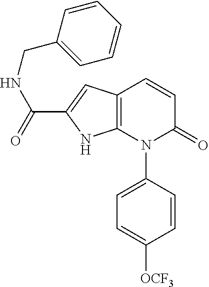 |
| 290 | 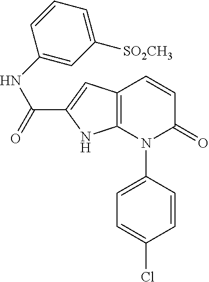 |
| 291 | 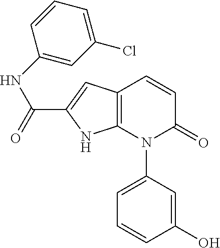 |
| 292 | 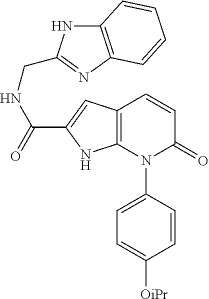 |
| 293 | 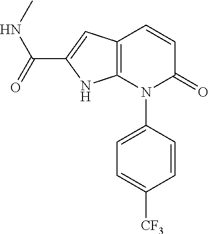 |
| 294 | 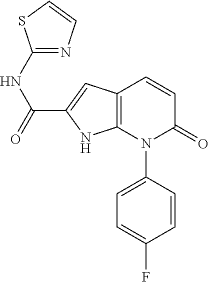 |
| 295 | 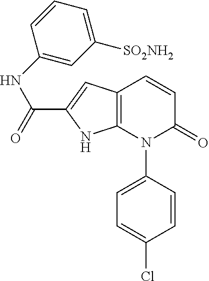 |
| 296 | 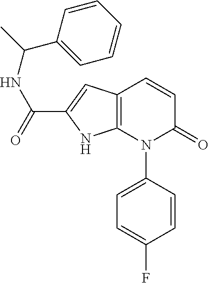 |
| 297 | 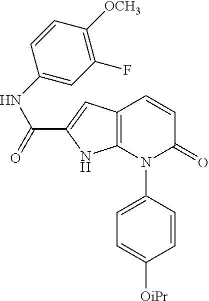 |
| 298 | 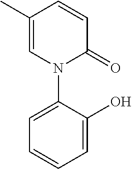 |
| 299 | 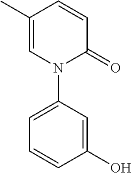 |
| 300 | 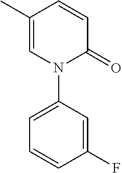 |
| 301 | 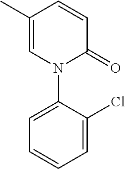 |
| 302 | 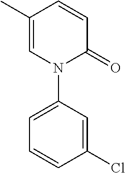 |
| 303 | 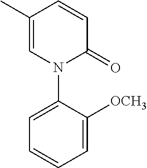 |
| 304 | 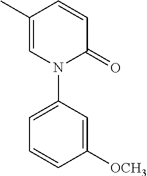 |
| 305 | 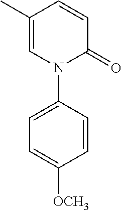 |
| 306 | 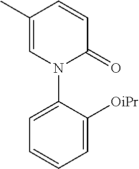 |
| 307 | 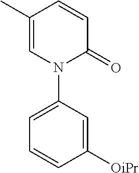 |
| 308 | 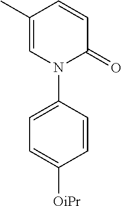 |
| 309 | 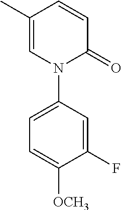 |
| 310 | 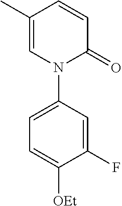 |
| 311 | 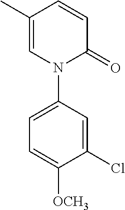 |
| 312 | 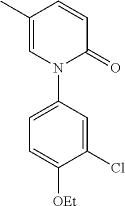 |
| 313 | 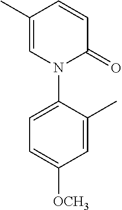 |
| 314 | 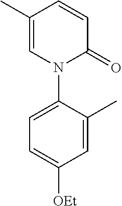 |
| 315 | 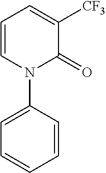 |
| 316 | 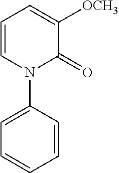 |
| 317 | 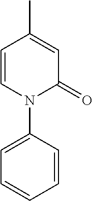 |
| 318 | 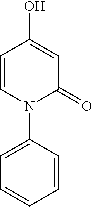 |
| 319 | 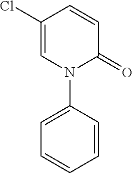 |
| 320 | 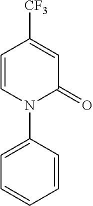 |
| 321 | 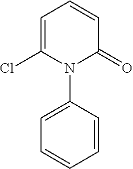 |
| 322 | 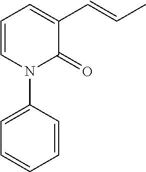 |
| 323 | 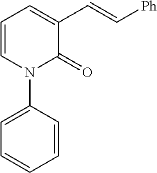 |
| 324 | 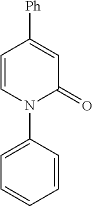 |
| 325 | 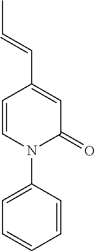 |
| 326 | 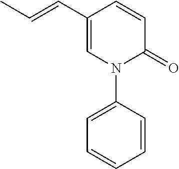 |
| 327 | 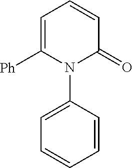 |
| 328 | 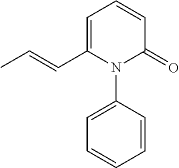 |
| 329 | 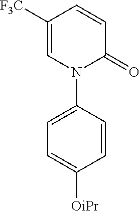 |
| 330 | 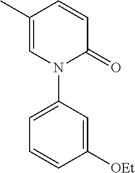 |
| 331 | 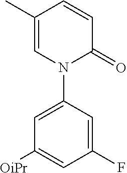 |
| 332 | 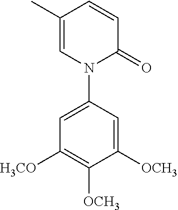 |
| 333 | 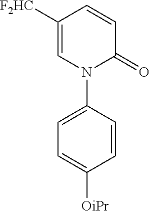 |
| 334 | 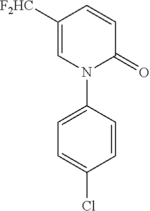 |
| 335 | 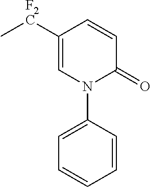 |
| 336 | 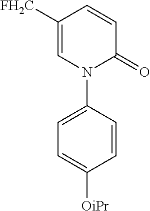 |
| 337 | 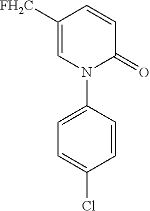 |
| 338 | 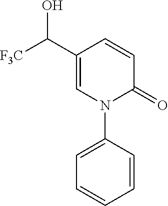 |
| 339 | 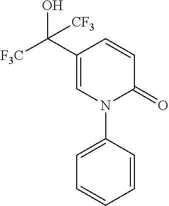 |
| 340 | 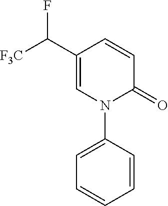 |
| 341 | 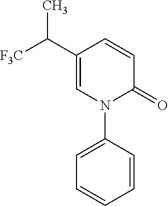 |
| 342 | 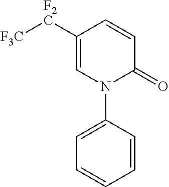 |
| 343 | 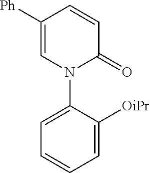 |
| 344 | 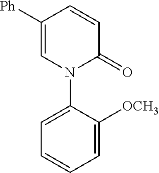 |
| 345 | 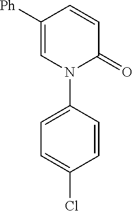 |
| 346 | 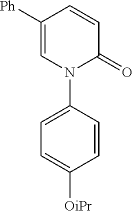 |
| 347 | 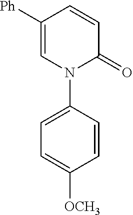 |
| 348 | 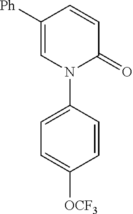 |
| 349 | 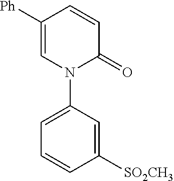 |
| 350 | 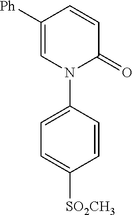 |
| 351 | 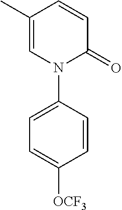 |
| 352 | 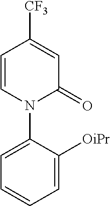 |
| 353 | 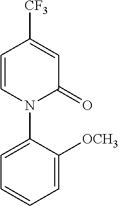 |
| 354 | 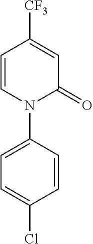 |
| 355 | 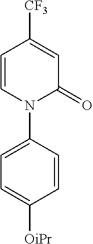 |
| 356 | 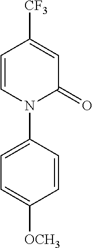 |
| 357 | 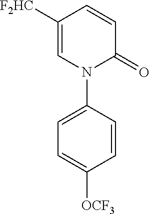 |
| 358 | 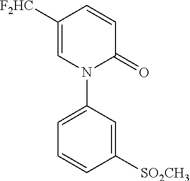 |
| 359 | 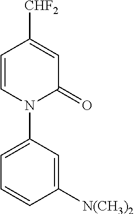 |
| 360 | 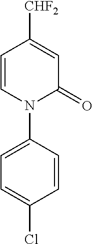 |
| 361 | 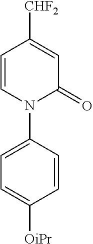 |
| 362 | 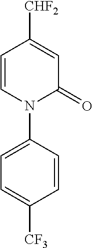 |
| 363 | 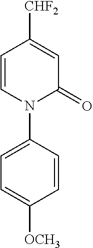 |
| 364 | 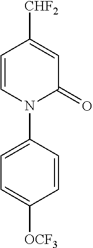 |
| 365 | 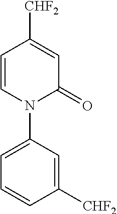 |
| 366 | 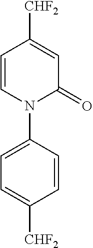 |
| 367 | 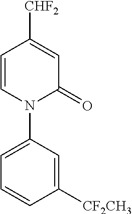 |
| 368 | 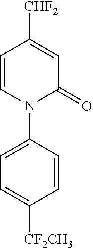 |
| 369 | 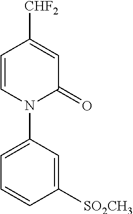 |
| 370 | 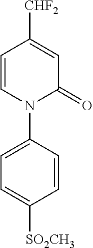 |
| 371 | 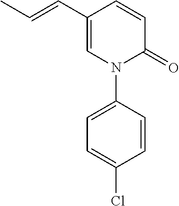 |
| 372 | 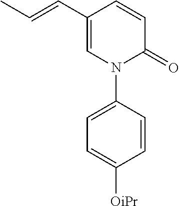 |
| 373 | 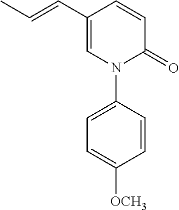 |
| 374 | 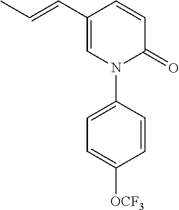 |
| 375 | Intentionally blank |
| 376 | 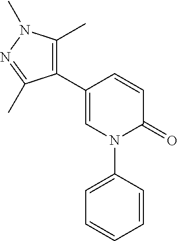 |
| 377 | 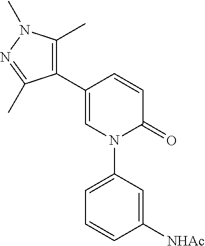 |
| 378 | 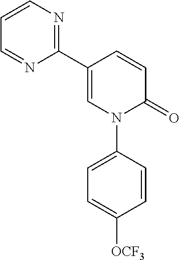 |
| 379 | 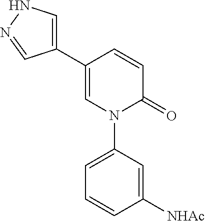 |
| 380 | 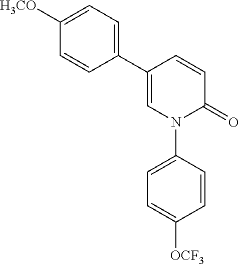 |
| 381 | 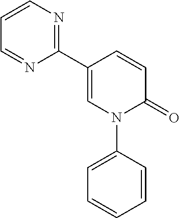 |
| 382 | 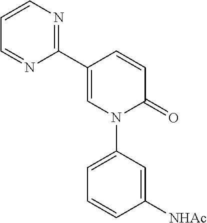 |
| 383 | 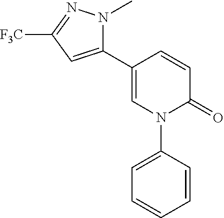 |
| 384 | 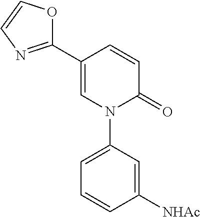 |
| 385 | 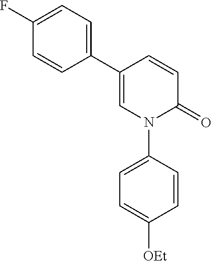 |
| 386 | 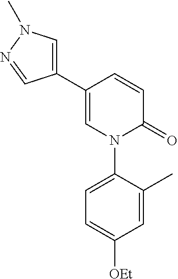 |
| 387 | 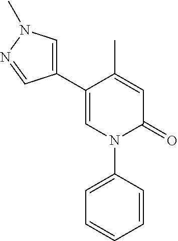 |
| 388 | 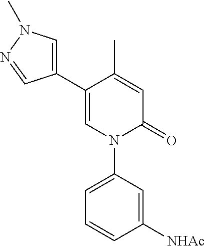 |
| 389 | 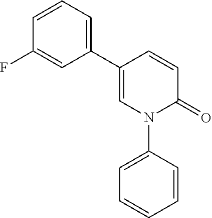 |
| 390 | 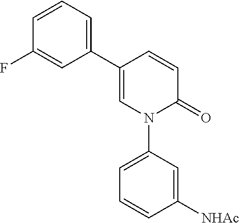 |
| 391 | 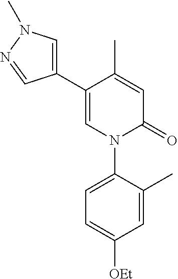 |
| 392 | 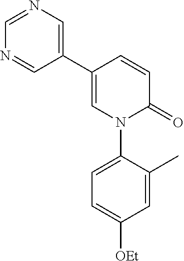 |
| 393 | 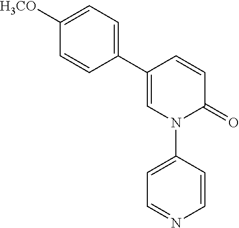 |
| 394 | 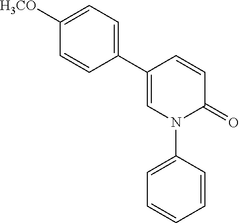 |
| 395 | 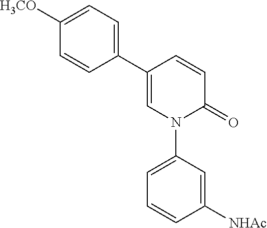 |
| 396 | 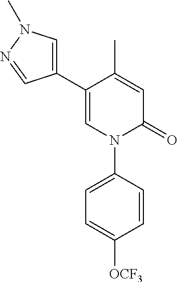 |
| 397 | 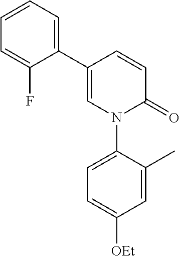 |
| 398 | 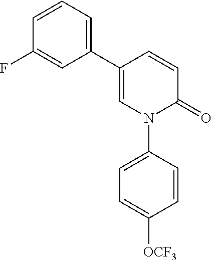 |
| 399 | 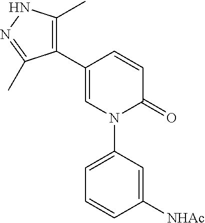 |
| 400 | 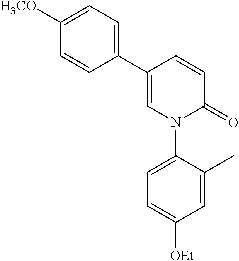 |
| 401 | 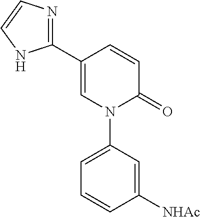 |
| 402 | 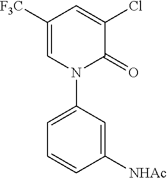 |
| 403 | 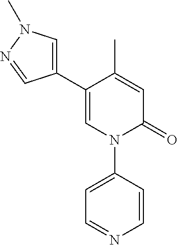 |
| 404 | 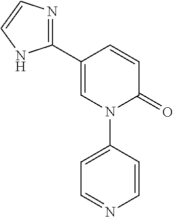 |
| 405 | 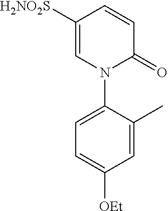 |
| 406 | 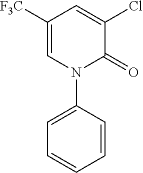 |
| 407 | 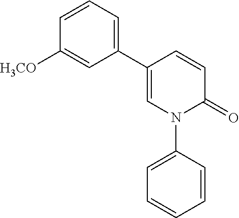 |
| 408 | 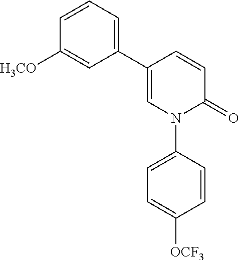 |
| 409 | 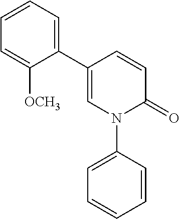 |
| 410 | 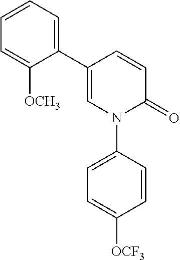 |
| 411 | 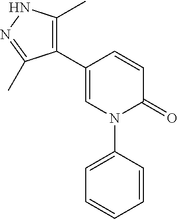 |
| 412 | 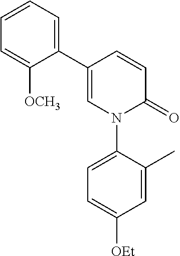 |
| 413 | 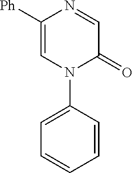 |
| 414 | 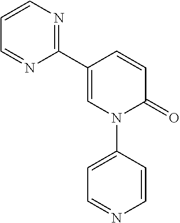 |
| 415 | 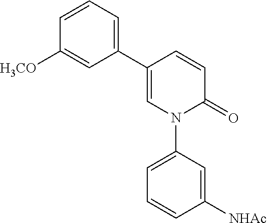 |
| 416 | 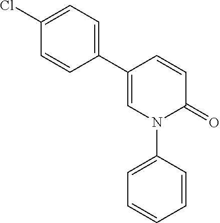 |
| 417 | 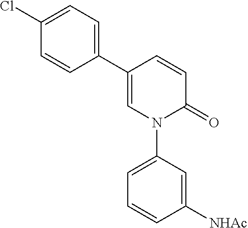 |
| 418 | 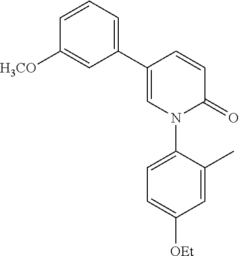 |
| 419 | 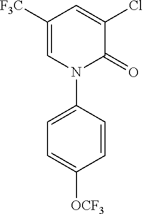 |
| 420 | 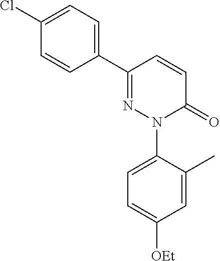 |
| 421 | 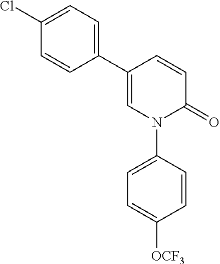 |
| 422 | 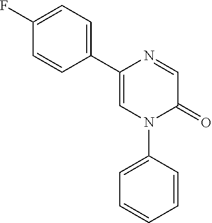 |
| 423 | 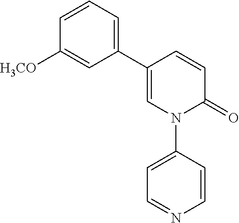 |
| 424 | 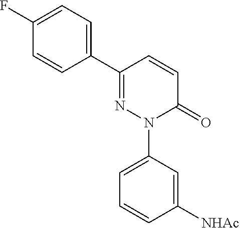 |
| 425 | 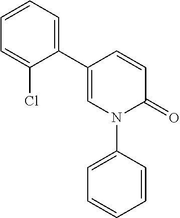 |
| 426 | 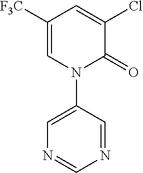 |
| 427 | 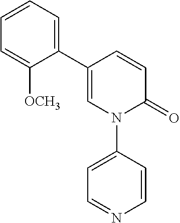 |
| 428 | 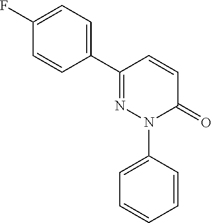 |
| 429 | 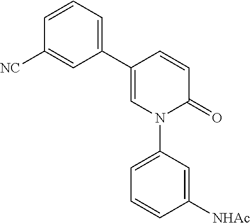 |
| 430 | 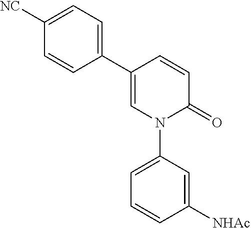 |
| 431 | 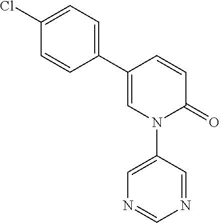 |
| 432 | 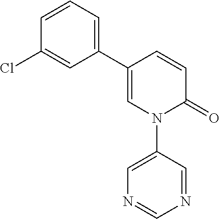 |
| 433 | 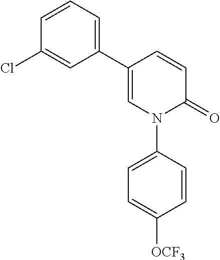 |
| 434 | 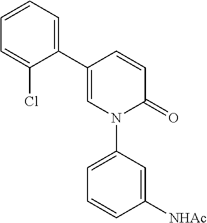 |
| 435 | 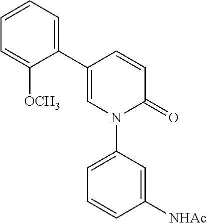 |
| 436 | 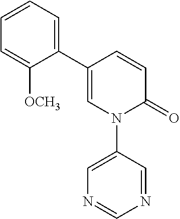 |
| 437 | 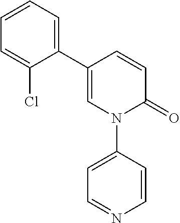 |
| 438 | 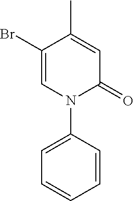 |
| 439 | 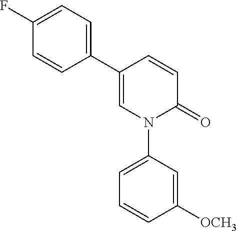 |
| 440 | 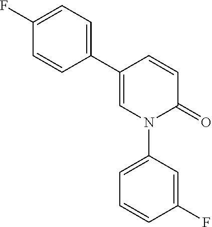 |
| 441 | 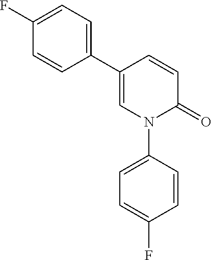 |
| 442 | 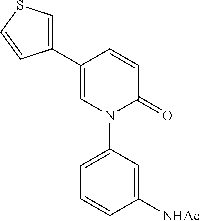 |
| 443 | 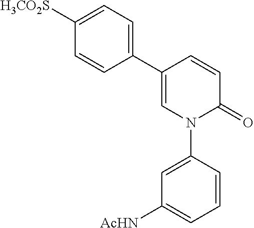 |
| 444 | 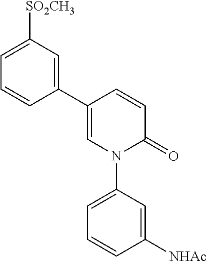 |
| 445 | 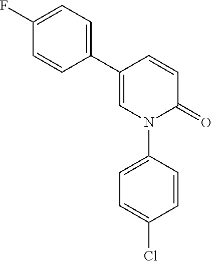 |
| 446 | 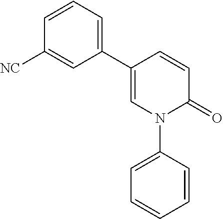 |
| 447 | 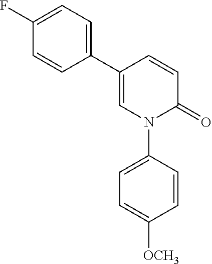 |
| 448 | 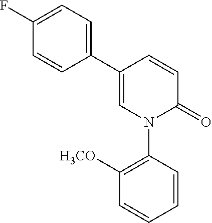 |
| 449 | 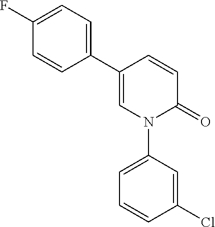 |
| 450 | 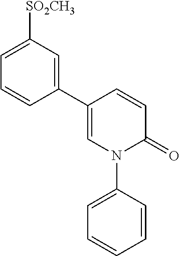 |
| 451 | 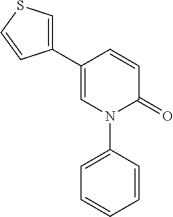 |
| 452 | 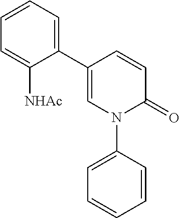 |
| 453 | 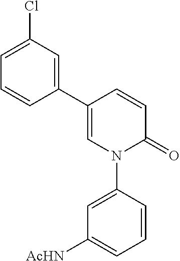 |
| 454 | 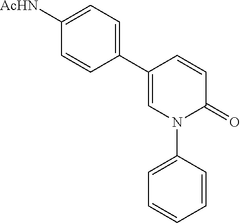 |
| 455 | 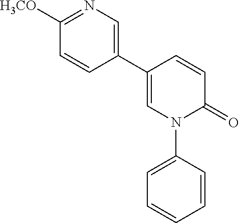 |
| 456 | 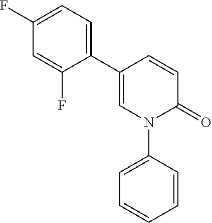 |
| 457 | 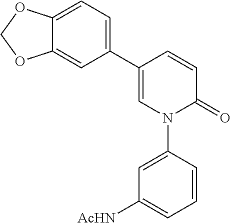 |
| 458 | 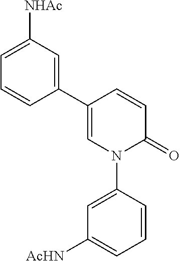 |
| 459 | 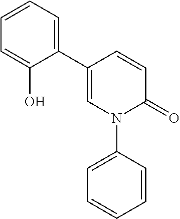 |
| 460 | 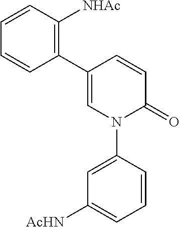 |
| 461 | 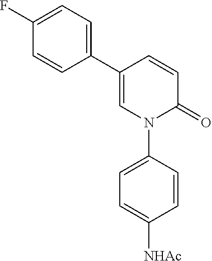 |
| 462 | 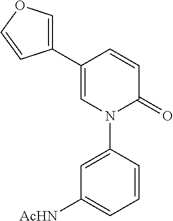 |
| 463 | 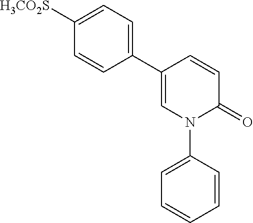 |
| 464 | 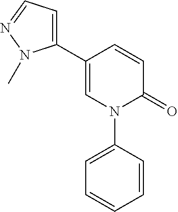 |
| 465 | 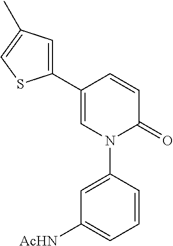 |
| 466 | 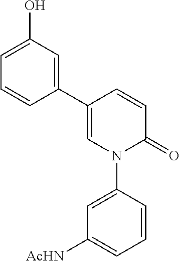 |
| 467 | 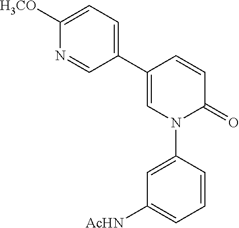 |
| 468 | 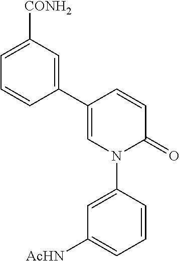 |
| 469 | 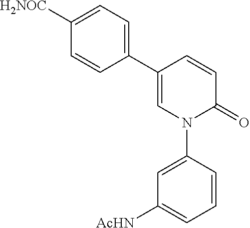 |
| 470 | 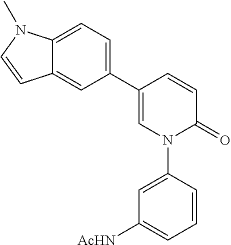 |
| 471 | 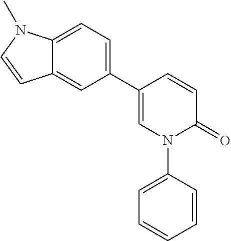 |
| 472 | 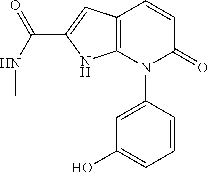 |
| 473 | 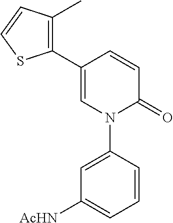 |
| 474 | 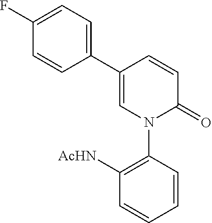 |
| 475 | 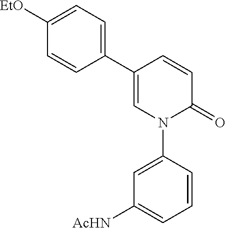 |
| 476 | 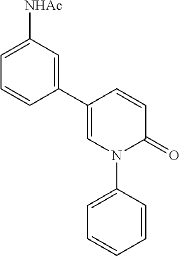 |
| 477 | 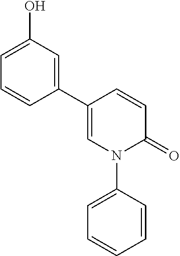 |
| 478 | 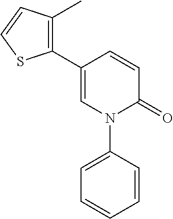 |
| 479 | 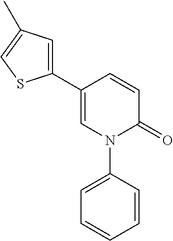 |
| 480 | 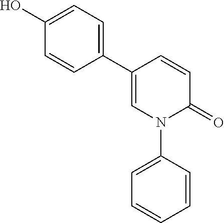 |
| 481 | 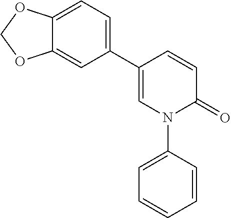 |
| 482 | 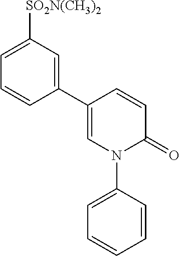 |
| 483 | 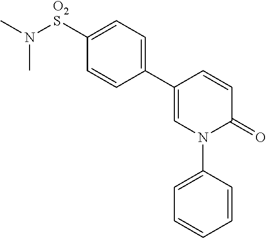 |
| 484 | 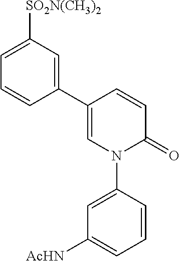 |
| 485 | 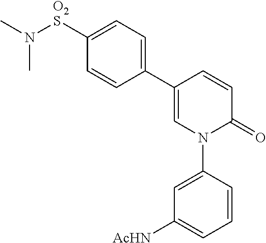 |
| 486 | 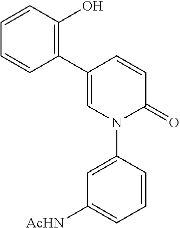 |
| 487 | 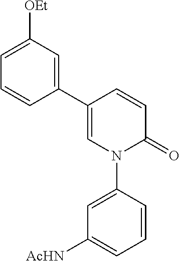 |

Other specific anti-fibrotic compounds of formula (I), (II), (III), or (IV) also include the following compounds.

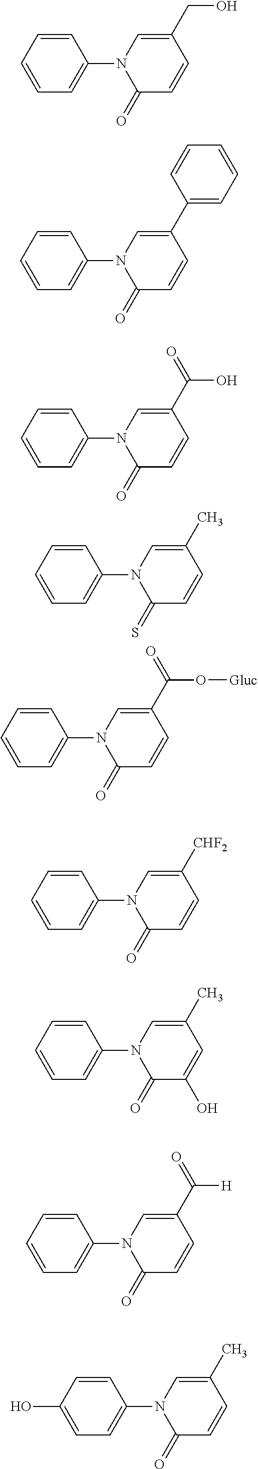

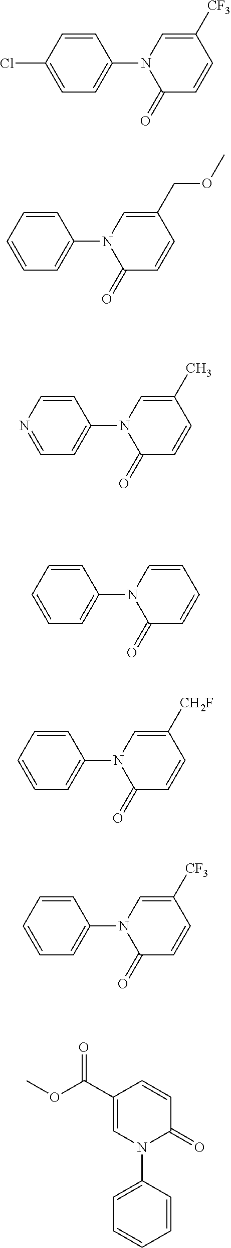

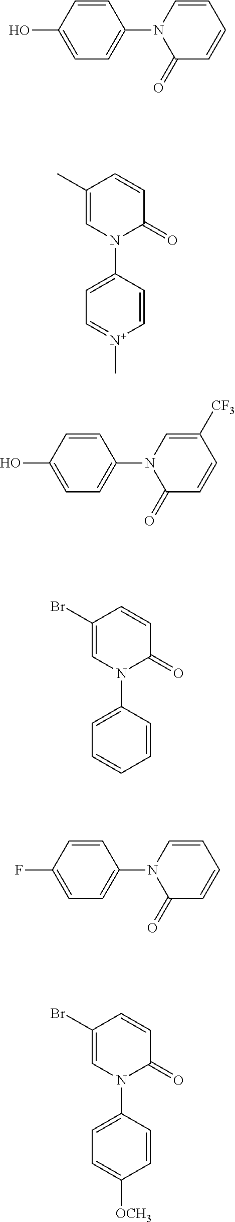

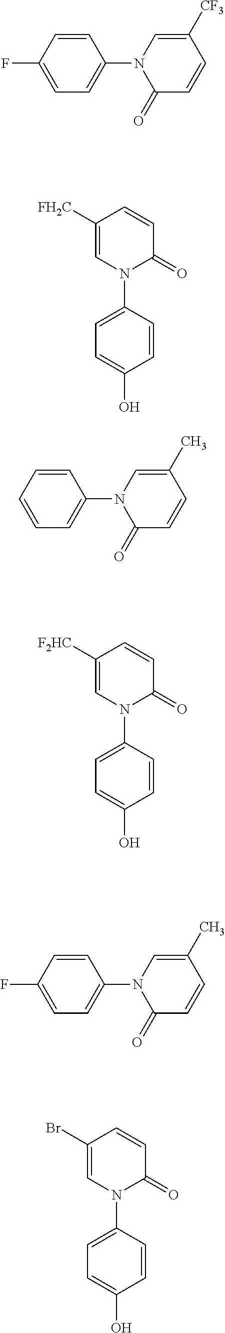

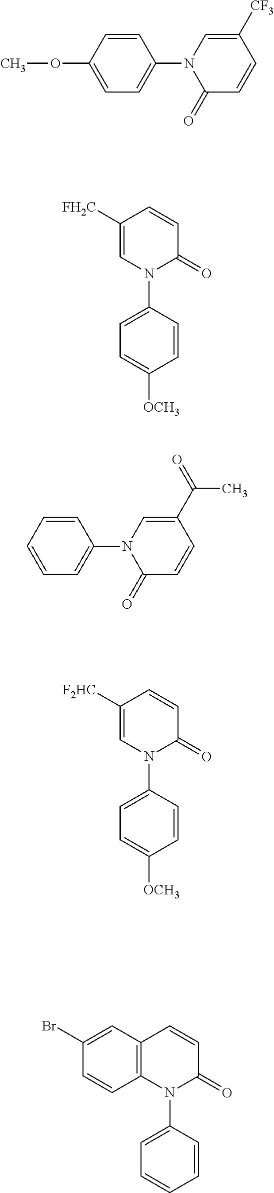

Other anti-fibrotic compounds contemplated for use in the disclosed methods include compounds of Genus I, II, III, and IV, below. Synthesis of compounds of Genus I, II, III, and IV are described in detail in International Patent Publication No. WO 07/062,167, incorporated by reference in its entirety herein.

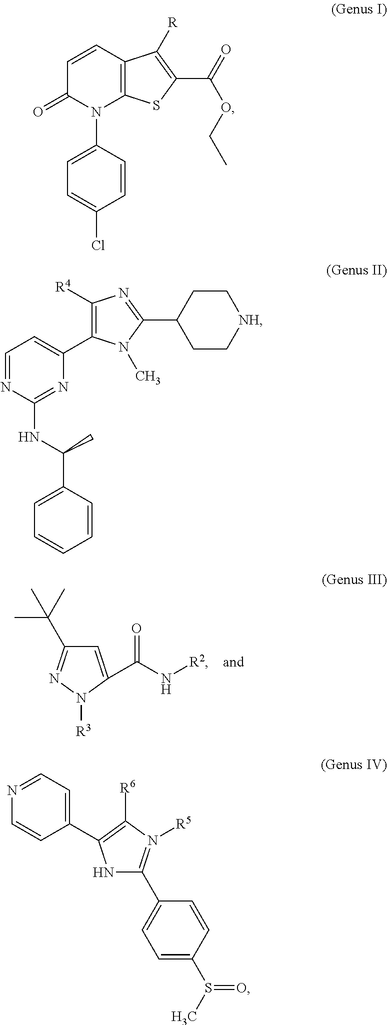

wherein each of R, R², R³, R⁴, R⁵, and R⁶ is independently selected from the group consisting of H, halo, cyano, nitro, hydroxy, optionally substituted C₁₋₆ alkyl, optionally substituted C₃₋₇ cycloalkyl, optionally substituted C₄₋₁₀ alkylcycloalkyl, optionally substituted C₂₋₆ alkenyl, optionally substituted C₁₋₆ alkoxy, optionally substituted C_{6 or 10} aryl, optionally substituted pyridinyl, optionally substituted pyrimidinyl, optionally substituted thienyl, optionally substituted furanyl, optionally substituted thiazolyl, optionally substituted oxazolyl, optionally substituted phenoxy, optionally substituted thiophenoxy, optionally substituted sulphonamido, optionally substituted urea, optionally substituted thiourea, optionally substituted amido, optionally substituted keto, optionally substituted carboxyl, optionally substituted carbamyl, optionally substituted sulphide, optionally substituted sulphoxide, optionally substituted sulphone, optionally substituted amino, optionally substituted alkoxyamino, optionally substituted alkyoxyheterocyclyl, optionally substituted alkylamino, optionally substituted alkylcarboxy, optionally substituted carbonyl, optionally substituted spirocyclic cycloalkyl, optionally substituted pyrazinyl, optionally substituted pyridazinyl, optionally substituted pyrrolyl, optionally substituted thiophenyl, optionally substituted thiazolyl, optionally substituted oxazolyl, optionally substituted imidazolyl, optionally substituted isoxazolyl, optionally substituted pyrazolyl, optionally substituted isothiazolyl, optionally substituted napthyl, optionally substituted quinolinyl, optionally substituted isoquinolinyl, optionally substituted quinoxalinyl, optionally substituted benzothiazolyl, optionally substituted benzothiophenyl, optionally substituted benzofuranyl, optionally substituted indolyl, and optionally substituted benzimidazolyl, or a pharmaceutically acceptable salt, ester, solvate or prodrug thereof.

The salts, e.g., pharmaceutically acceptable salts, of the disclosed therapeutics may be prepared by reacting the appropriate base or acid with a stoichiometric equivalent of the therapeutic. Similarly, pharmaceutically acceptable derivatives (e.g., esters), metabolites, hydrates, solvates and prodrugs of the therapeutic may be prepared by methods generally known to those skilled in the art. Thus, another embodiment provides compounds that are prodrugs of an active compound. In general, a prodrug is a compound which is metabolized in vivo (e.g., by a metabolic transformation such as deamination, dealkylation, de-esterification, and the like) to provide an active compound. A “pharmaceutically acceptable prodrug” means a compound which is, within the scope of sound medical judgment, suitable for pharmaceutical use in a patient without undue toxicity, irritation, allergic response, and the like, and effective for the intended use, including a pharmaceutically acceptable ester as well as a zwitterionic form, where possible, of the therapeutic. As used herein, the term “pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Representative examples of particular esters include, but are not limited to, formates, acetates, propionates, butyrates, acrylates and ethylsuccinates. Examples of pharmaceutically-acceptable prodrug types are described in Higuchi and Stella, Pro-drugs as Novel Delivery Systems, Vol. 14 of the A.C.S. Symposium Series, and in Roche, ed., Bioreversible Carriers in Drug Design, American Pharmaceutical Association and Pergamon Press, 1987, both of which are incorporated herein by reference.

The compounds and compositions described herein may also include metabolites. As used herein, the term “metabolite” means a product of metabolism of a compound of the embodiments or a pharmaceutically acceptable salt, analog, or derivative thereof, that exhibits a similar activity in vitro or in vivo to a disclosed therapeutic. The compounds and compositions described herein may also include hydrates and solvates. As used herein, the term “solvate” refers to a complex formed by a solute (herein, the therapeutic) and a solvent. Such solvents for the purpose of the embodiments preferably should not negatively interfere with the biological activity of the solute. Solvents may be, by way of example, water, ethanol, or acetic acid. In view of the foregoing, reference herein to a particular compound or genus of compounds will be understood to include the various forms described above, including pharmaceutically acceptable salts, esters, prodrugs, metabolites and solvates thereof.

Additional anti-fibrotic agents contemplated for use in the methods of the present disclosure can be any agent that affects fibrosis. Contemplated agents include, but are not limited to, those that reduce the activity of transforming growth factor-beta (TGF-β) (including but not limited to GC-1008 (Genzyme/MedImmune); lerdelimumab (CAT-152; Trabio, Cambridge Antibody); metelimumab (CAT-192, Cambridge Antibody,); LY-2157299 (Eli Lilly); ACU-HTR-028 (Opko Health)) including antibodies that target one or more TGF-β isoforms, inhibitors of TGF-β receptor kinases TGFBR1 (ALK5) and TGFBR2, and modulators of post-receptor signaling pathways; chemokine receptor signaling; endothelin receptor antagonists including inhibitors that target both endothelin receptor A and B and those that selectively target endothelin receptor A (including but not limited to ambrisentan; avosentan; bosentan; clazosentan; darusentan; BQ-153; FR-139317, L-744453; macitentan; PD-145065; PD-156252; PD163610; PS-433540; S-0139; sitaxentan sodium; TBC-3711; zibotentan); agents that reduce the activity of connective tissue growth factor (CTGF) (including but not limited to FG-3019, FibroGen), and also including other CTGF-neutralizing antibodies; matrix metalloproteinase (MMP) inhibitors (including but not limited to MMPI-12, PUP-1 and tigapotide triflutate); agents that reduce the activity of epidermal growth factor receptor (EGFR) including but not limed to erlotinib, gefitinib, BMS-690514, cetuximab, antibodies targeting EGF receptor, inhibitors of EGF receptor kinase, and modulators of post-receptor signaling pathways; agents that reduce the activity of platelet derived growth factor (PDGF) (including but not limited to Imatinib mesylate (Novartis)) and also including PDGF neutralizing antibodies, antibodies targeting PDGF receptor (PDGFR), inhibitors of PDGFR kinase activity, and post-receptor signaling pathways; agents that reduce the activity of vascular endothelial growth factor (VEGF) (including but not limited to axitinib, bevacizumab, BIBF-1120, CDP-791, CT-322, IMC-18F1, PTC-299, and ramucirumab) and also including VEGF-neutralizing antibodies, antibodies targeting the VEGF receptor 1 (VEGFR1, Flt-1) and VEGF receptor 2 (VEGFR2, KDR), the soluble form of VEGFR1 (sFlt) and derivatives thereof which neutralize VEGF, and inhibitors of VEGF receptor kinase activity; inhibitors of multiple receptor kinases such as BIBF-1120 which inhibits receptor kinases for vascular endothelial growth factor, fibroblast growth factor, and platelet derived growth factor; agents that interfere with integrin function (including but not limited to STX-100 and IMGN-388) and also including integrin targeted antibodies; agents that interfere with the pro-fibrotic activities of IL-4 (including but not limited to AER-001, AMG-317, APG-201, and sIL-4Rα) and IL-13 (including but not limited to AER-001, AMG-317, anrukinzumab, CAT-354, cintredekin besudotox, MK-6105, QAX-576, SB-313, SL-102, and TNX-650) and also including neutralizing anti-bodies to either cytokine, antibodies that target IL-4 receptor or IL-13 receptor, the soluble form of IL-4 receptor or derivatives thereof that is reported to bind and neutralize both IL-4 and IL-13, chimeric proteins including all or part of IL-13 and a toxin particularly pseudomonas endotoxin, signaling though the JAK-STAT kinase pathway; agents that interfere with epithelial mesenchymal transition including inhibitors of mTor (including but not limited to AP-23573); agents that reduce levels of copper such as tetrathiomolybdate; agents that reduce oxidative stress including N-acetyl cysteine and tetrathiomolybdate; and interferon gamma. Also contemplated are agents that are inhibitors of phosphodiesterase 4 (PDE4) (including but not limited to Roflumilast); inhibitors of phosphodiesterase 5 (PDE5) (including but not limited to mirodenafil, PF-4480682, sildenafil citrate, SLx-2101, tadalafil, udenafil, UK-369003, vardenafil, and zaprinast); or modifiers of the arachidonic acid pathway including cyclooxygenase and 5-lipoxegenase inhibitors (including but not limited to Zileuton). Further contemplated are compounds that reduce tissue remodeling or fibrosis including prolyl hydrolase inhibitors (including but not limited to 1016548, CG-0089, FG-2216, FG-4497, FG-5615, FG-6513, fibrostatin A (Takeda), lufironil, P-1894B, and safironil) and peroxisome proliferator-activated receptor (PPAR)-gamma agonists. (including but not limited to pioglitazone and rosiglitazone).

Other specific anti-fibrotic agents contemplated include relaxin, ufironil, surifonil, a TGF-β antibody, CAT-192, CAT-158; ambresentan, thelin; FG-3019, a CTGF antibody; anti-EGFR antibody; a EGFR kinase inhibitor; tarceva; gefitinib; PDGF antibody, PDGFR kinase inhibitor; gleevec; BIBF-1120, VEGF, FGF, and PDGF receptor inhibitor; anti-integrin antibody; IL-4 antibody; tetrathiomolybdate, a copper chelating agent; interferon-gamma; NAC, a cysteine pro-drug; hepatocyte growth factor (HGF); KGF; angiotension receptor blockers, ACE inhibitors, rennin inhibitors; COX and LO inhibitors; Zileuton; monteleukast; avastin; statins; PDE5 inhibitors, such as sildenafil, udenafil, tadalafil, vardenafil, or zaprinast; rofumilast; etanercept (Enbrel); procoagulant; prostaglandins, such as PGE2, PRX-08066, a 5HT2B receptor antagonist; cintredekin besudotox, a chimeric human IL13 conjugated to a genetically engineered Pseudomonas exotoxin; roflumilast, a PDE4 inhibitor; FG-3019, an anti-connective tissue growth factor human monoclonal antibody; GC-1008, a TGF-β human monoclonal antibody; treprostinil, a prostacyclin analog; interferon-α; QAX-576, a IL13 modulator; WEB 2086, a PAF-receptor antagonist; imatinib mesylate; FG-1019; Suramin; Bosentan; IFN-1b; anti-IL-4; anti-IL-13; taurine, niacin, NF-κB antisense oligonucleotides; and nitric oxide synthase inhibitors.

### HIV Therapeutic Agents

One group of HIV therapeutic agents contemplated include nucleoside reverse transcriptase inhibitors, non-nucleoside reverse transcriptase inhibitors, protease inhibitors, CCR5 antagonists, integrase inhibitors and fusion inhibitors.

Nucleoside Reverse Transcriptase Inhibitors (NRTIs) contemplated by the invention include, but are not limited to, Abacavir (ABC) (ZIAGEN), TRIZIVIR, EPZICOM, Didanosine (ddI) (VIDEX EC), Emtricitabine (EMTRIVA), ATRIPLA, TRUVADA, Lamivudine (EPIVIR), COMBIVIR, EPZICOM, Stavudine (ZERIT), Tenofovir Disoproxil Fumarate (VIREAD), ATRIPLA, TRUVADA, and Zidovudine (RETROVIR).

Non-Nucleoside Reverse Transcriptase Inhibitors (NNRTIs) contemplated by the invention include, but are not limited to, Delavirdine (RESCRIPTOR), Efavirenz (SUSTIVA), Etravirine (INTELENCE), and Nevirapine (VIRAMUNE).

Protease Inhibitors (PIs) contemplated by the invention, but are not limited to, include Atazanavir (REYATAZ), Darunavir (PREZISTA), Fosamprenavir (LEXIVA), Indinavir (CRIXIVAN), Lopinavir+Ritonavir (KALETRA), Nelfinavir (VIRACEPT), Ritonavir (NORVIR), Saquinavir (INVIRASE), and Tipranavir (APTIVUS).

A Fusion Inhibitor contemplated by the invention is Enfuvirtide (FUZEON).

A CCR5 Antagonist contemplated by the invention is Maraviroc (SELZENTRY).

An Integrase Inhibitor contemplated by the invention is Raltegravir (ISENTRESS).

HIV therapeutics can also include non-antiretroviral therapeutics. Examples include, but are not limited to atovaquone (MEPRON, BW566C80), azithromycin (ZITHROMAX), Bactrim (SEPTRA, TMP/SMX), ciprofloxacin (CIPRO), clarithromycin (BIAXIN), co-enzyme Q, colony stimulating factor (G-CSF, NEUPOGEN), dapsone, DHEA (dihydroepiandrostenedione), erythropoietin (EPOGEN, PROCRIT), ethambutol (MYAMBUTOL), fluconazole (DIFLUCAN), foscarnet (FOSCAVIR), ganciclovir (CYTOVENE, DHPG, valcyte, valganciclovir), interleukin 2 (IL-2), dronabinol (MARINOL), MEGACE (megestrol acetate), NAC (N-acetyl cysteine), deca-durabolin (DURABOLIN, NANDROLONE), oxandrolone (OXANDRIN, ANAVAR), rifabutin (MYCOBUTIN, ANSAMYCIN), human growth hormone (SEROSTIM), testosterone (ANDROGEL, ANDRODERM, DELATESTRYL, TESTODERM, VIRILON). Preferably, the non-antiretroviral therapeutic is one that boosts CD4 cell count, including but not limited to interleukins (e.g., Interleukin-2, Interleukin-7).

Combination therapy using combinations of HIV therapeutic agents is also contemplated for use in the methods described herein, and is within the skill in the art of the clinician to determine.

### Dosing and Pharmaceutical Formulations

The anti-fibrotic agents disclosed herein can be dosed at a total amount of about 1 to about 4800 mg per day, or about 30 to about 3600 mg to day, or about 50 to about 2400 mg per day, for example 2403 mg per day. The dosage can be divided, for example into two or three doses over the day, or can be given in a single daily dose. Specific amounts of the total daily amount of the therapeutic contemplated for the disclosed methods include about 50 mg, about 100 mg, about 150 mg, about 200 mg, about 250 mg, about 267 mg, about 300 mg, about 350 mg, about 400 mg, about 450 mg, about 500 mg, about 534 mg, about 550 mg, about 600 mg, about 650 mg, about 700 mg, about 750 mg, about 800 mg, about 850 mg, about 900 mg, about 950 mg, about 1000 mg, about 1050 mg, about 1068 mg, about 1100 mg, about 1150 mg, about 1200 mg, about 1250 mg, about 1300 mg, about 1335 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1869 mg, about 1900 mg, about 1950 mg, about 2000 mg, about 2050 mg, about 2100 mg, about 2136 mg, about 2150 mg, about 2200 mg, about 2250 mg, about 2300 mg, about 2350 mg, about 2400 mg, and about 2403 mg. The foregoing amounts and ranges are contemplated for pirfenidone and pirfenidone analogs. Additionally, dosing regimens for the anti-fibrotic agents are contemplated. Dosing regimens for use in the methods of the present disclosure can be selected from those disclosed in U.S. Pat. No. 7,696,236 (which is incorporated herein by reference in its entirety).

Dosages of the anti-fibrotic agent can alternately be administered as a dose measured in mg active agent per kg body weight. Contemplated mg/kg doses of the disclosed therapeutics include about 1 mg/kg to about 60 mg/kg. Specific ranges of doses in mg/kg include about 1 mg/kg to about 20 mg/kg, about 5 mg/kg to about 20 mg/kg, about 10 mg/kg to about 20 mg/kg, about 25 mg/kg to about 50 mg/kg, and about 30 mg/kg to about 60 mg/kg.

The HIV therapeutic agents disclosed herein can be administered according to the Guidelines for the Use of Antiretroviral Agents in HIV-1-Infected Adults and Adolescents [Panel on Antiretroviral Guidelines for Adults and Adolescents, Guidelines for the use of antiretroviral agents in HIV-1-infected adults and adolescents, Department of Health and Human Services. Nov. 3, 2008; 1-139]. According to these Guidelines, antiretroviral therapy is to be initiated in patients with a history of an AIDS-defining illness or with a CD4 T-cell count less than 350 cells/mm³. The data supporting this recommendation are stronger for those with a CD4 T-cell count less than 200 cells/mm³ and with a history of AIDS than for those with CD4 T-cell counts between 200 and 350 cells/mm³. Antiretroviral therapy should also be initiated in the following groups of patients regardless of CD4 T-cell count: (a) pregnant women; (b) patients with HIV-associated nephropathy; and (c) patients coinfected with hepatitis B virus (HBV) when treatment of HBV is indicated. The guidelines are summarized in Table 2, below.

TABLE 2

| CLINICAL CONDITION AND/OR CD4 COUNT | RECOMMENDATIONS |
|---|---|
| History of AIDS-defining | ANTIRETROVIRAL THERAPY SHOULD BE |
| illness | INITIATED. |
| CD4 count <200 cells/mm³ | |
| CD4 count 200-350 cells/mm³ | |
| Pregnant women* | |
| Persons with HIV-associated | |
| nephropathy | |
| Persons coinfected with | |
| hepatitis B virus (HBV), when | |
| HBV treatment is indicated | |
| (Treatment with fully | |
| suppressive antiviral drugs | |
| active against both HIV and | |
| HBV is recommended.) | |
| PATIENTS WITH CD4 COUNT >350 | THE OPTIMAL TIME TO INITIATE THERAPY IN |
| CELLS/MM³ WHO DO NOT MEET | ASYMPTOMATIC PATIENTS WITH CD4 COUNT |
| ANY OF THE SPECIFIC | >350 CELLS/MM³ IS NOT WELL DEFINED. |
| CONDITIONS LISTED ABOVE. | PATIENT SCENARIOS AND COMORBIDITIES |
| | SHOULD BE TAKEN INTO CONSIDERATION. |

*For women who do not require antiretroviral therapy for their own health, consideration can be given to discontinuing antiretroviral drugs postpartum.

Antiretroviral therapy may be considered in some patients with CD4 T-cell counts greater than 350 cells/mm³. The guidelines to be considered are outlined in Table 3, below.

TABLE 3

| POTENTIAL BENEFITS OF EARLY THERAPY | POTENTIAL RISKS OF EARLY THERAPY |
|---|---|
| INCLUDE: | INCLUDE: |
| MAINTENANCE OF A HIGHER CD4 COUNT | DEVELOPMENT OF TREATMENT-RELATED |
| AND PREVENTION OF POTENTIALLY | SIDE EFFECTS AND TOXICITIES |
| IRREVERSIBLE DAMAGE TO THE IMMUNE | DEVELOPMENT OF DRUG RESISTANCE |
| SYSTEM | BECAUSE OF INCOMPLETE VIRAL SUPPRESSION, |
| DECREASED RISK FOR HIV-ASSOCIATED | RESULTING IN LOSS OF FUTURE TREATMENT |
| COMPLICATIONS THAT CAN SOMETIMES OCCUR | OPTIONS |
| AT CD4 COUNTS >350 CELLS/MM³, INCLUDING | LESS TIME FOR THE PATIENT TO LEARN |
| TUBERCULOSIS, NON-HODGKIN'S LYMPHOMA, | ABOUT HIV AND ITS TREATMENT AND LESS |
| KAPOSI'S SARCOMA, PERIPHERAL | TIME TO PREPARE FOR THE NEED FOR |
| NEUROPATHY, HPV-ASSOCIATED | ADHERENCE TO THERAPY |
| MALIGNANCIES, AND HIV-ASSOCIATED | INCREASED TOTAL TIME ON MEDICATION, |
| COGNITIVE IMPAIRMENT | WITH GREATER CHANCE OF TREATMENT |
| DECREASED RISK OF NONOPPORTUNISTIC | FATIGUE |
| CONDITIONS, INCLUDING CARDIOVASCULAR | PREMATURE USE OF THERAPY BEFORE THE |
| DISEASE, RENAL DISEASE, LIVER DISEASE, AND | DEVELOPMENT OF MORE EFFECTIVE, LESS |
| NON-AIDS-ASSOCIATED MALIGNANCIES AND | TOXIC, AND/OR BETTER STUDIED |
| INFECTIONS | COMBINATIONS OF ANTIRETROVIRAL DRUGS |
| DECREASED RISK OF HIV TRANSMISSION TO | TRANSMISSION OF DRUG-RESISTANT VIRUS |
| OTHERS, WHICH WILL HAVE POSITIVE PUBLIC | IN PATIENTS WHO DO NOT MAINTAIN FULL |
| HEALTH IMPLICATIONS | VIROLOGIC SUPPRESSION |

An updated set of guidelines has also been made available [Guidelines for the Use of Antiretroviral Agents in HIV-1-Infected Adults and Adolescents (Developed by the DHHS Panel on Antiretroviral Guidelines for Adults and Adolescents—A Working Group of the Office of AIDS Research Advisory Council (OARAC)), Dec. 1, 2009]. The updated guidelines recommend the following with respect to initiation of ARV.
In this updated version of the guidelines, the Panel recommends earlier initiation of antiretroviral therapy with the following specific recommendations:
Antiretroviral therapy should be initiated in all patients with a history of an AIDS-defining illness or with CD4 count <350 cells/mm³ (AI).
Antiretroviral therapy should also be initiated, regardless of CD4 count, in patients with the following conditions: pregnancy (AI), HIV-associated nephropathy (AII), and hepatitis B virus (HBV) coinfection when treatment of HBV is indicated (AIII).
Antiretroviral therapy is recommended for patients with CD4 counts between 350 and 500 cells/mm³. The Panel was divided on the strength of this recommendation: 55% of Panel members for strong recommendation (A) and 45% for moderate recommendation (B) (A/B-II).
For patients with CD4 counts >500 cells/mm³, 50% of Panel members favor starting antiretroviral therapy (B); the other 50% of members view treatment as optional (C) in this setting (B/C-III).
Patients initiating antiretroviral therapy should be willing and able to commit to lifelong treatment and should understand the benefits and risks of therapy and the importance of adherence (AIII). Patients may choose to postpone therapy, and providers may elect to defer therapy, based on clinical and/or psychosocial factors on a case-by-case basis.

Accordingly, timing of HIV antiretroviral therapy relative to administration of the anti-fibrotic agents disclosed herein is a consideration. In one embodiment, a method of treating a patient diagnosed with human immunodeficiency virus (HIV) is provided comprising the step of administering to said patient a therapeutically effective amount of an anti-fibrotic agent, preferably pirfenidone or a compound described herein, and a HIV therapeutic agent, said amount of anti-fibrotic effective to decrease fibrosis in lymphatic tissue in said patient relative to a patient that is not treated with the anti-fibrotic agent, and said amount of the HIV therapeutic agent effective to increase CD4⁺ T cells in said patient relative to a patient that is not treated with the HIV therapeutic agent.

In another embodiment, a method of treating a patient diagnosed with HIV is provided comprising the step of administering to said patient a therapeutically effective amount of an anti-fibrotic agent, preferably pirfenidone or a compound described herein, and an HIV therapeutic agent, wherein said administering commences while the patient has a T cell count of at least 350 cells per mm³, or greater than 350 cells per mm³ In specific embodiments, the patient will be one who is a pregnant woman, one with HIV-associated nephropathy, one who is coinfected with HBV, or one having any combination of the foregoing three factors. In another embodiment, a method of treating a patient diagnosed with HIV is provided comprising the step of administering to said patient a therapeutically effective amount of an anti-fibrotic agent, preferably pirfenidone or a compound described herein, and an HIV therapeutic agent, wherein said administering commences while the patient has a T cell count below 350 cells/mm³.

In yet another embodiment, a method of treating a patient diagnosed with HIV is provided comprising the step of administering to said patient a therapeutically effective amount of an anti-fibrotic agent (preferably pirfenidone or a compound described herein) in the absence of treatment with a HIV therapeutic agent, prior to administering an anti-fibrotic agent (preferably pirfenidone or a compound described herein) in combination with HIV therapeutic agent. The timing of administration of the anti-fibrotic agent without HIV therapeutic agent relative to the commencement of administration of the anti-fibrotic agent with HIV therapeutic agent can be determined, for example, by CD4⁺ T cell counts and measurements of degree of fibrosis, for example using methods known in the art as well as described herein for such counts and measurements. In one embodiment, the anti-fibrotic agent is administered to a patient diagnosed with HIV when the percent area of T cell zone fibrosis is at least about 5%. In various aspects, the anti-fibrotic agent is administered to a patient diagnosed with HIV when the percent area of T cell zone fibrosis is at least 6%, or at least 7%, or at least 8%, or at least 9%, or at least 10%, or at least 11%, or at least 12%, or at least 13%, or at least 14%, or at least 15%, or at least 16%, or at least 17%, or at least 18%, or at least 19%, or at least 20%, or at least 25%, or at least 30%, or at least 35%, or at least 40% or higher. In other aspects, the commencement of administration of the anti-fibrotic agent with HIV therapeutic agent to a patient diagnosed with HIV is determined by the appropriateness of treatment. The appropriateness of treatment is determined by factors including but not limited to the Guidelines for the Use of Antiretroviral Agents as described herein and any severe (Grade II or worse) adverse drug interaction. Appropriateness of treatment can be determined by the clinician of skill in the art.

In another embodiment, it is contemplated that the anti-fibrotic agent is administered to a patient diagnosed with HIV when the percent area of the T cell zone occupied by CD4⁺ cells is at least about 50%. In various aspects, the anti-fibrotic agent is administered to a patient diagnosed with HIV when the percent area of the T cell zone occupied by CD4⁺ cells is at least about 49%, or at least 48%, or at least 47%, or at least 46%, or at least 45%, or at least 44%, or at least 43%, or at least 42%, or at least 41%, or at least 40%, or at least 39%, or at least 38%, or at least 37%, or at least 36%, or at least 35%, or at least 34%, or at least 33%, or at least 32%, or at least 31%, or at least 30%, or at least 29%, or at least 28%, or at least 27%, or at least 26%, or at least 25%, or at least 20%, or at least 15%, or at least 10%, or at least 5%, or lower.

It will be understood that in some embodiments of the invention, both percent area of T cell zone fibrosis and percent area of the T cell zone occupied by CD4⁺ cells will be calculated, and therefore that both quantities can be used to determine the timing of commencement of administration of an anti-fibrotic agent as disclosed herein. It is also contemplated that the timing of commencement of administration of an anti-fibrotic agent and a HIV therapeutic agent is within the skill of the art of the clinician to determine in view of the disclosure herein. For example, this can occur when the CD4⁺ T cell count is below 350 cells per mm³ (e.g., see Tables 2 and 3).

It is contemplated that dosages and timing of HIV therapeutic administration can vary between adult/adolescent patients and pediatric patients. For pediatric administration, the HIV therapeutic agents disclosed herein can be dosed according to the Guidelines for the Use of Antiretroviral Agents in Pediatric HIV Infection [Working Group on Antiretroviral Therapy and Medical Management of HIV-Infected Children, Guidelines for the Use of Antiretroviral Agents in Pediatric HIV Infection, Feb. 23, 2009; pp 1-139].

As disclosed by the above-referenced guidelines, a number of factors may need to be considered in making decisions about initiating and changing antiretroviral therapy in children, including: severity of HIV disease and risk of disease progression, as determined by age, presence or history of HIV-related or AIDS-defining illnesses, level of CD4 cell immunosuppression, and magnitude of HIV plasma viremia; availability of appropriate (and palatable) drug formulations and pharmacokinetic information on appropriate dosing in the child's age group; potency, complexity (e.g., dosing frequency, food and fluid requirements), and potential short- and long-term adverse effects of the antiretroviral regimen; effect of initial regimen choice on later therapeutic options; presence of comorbidity that could affect drug choice, such as tuberculosis, hepatitis B or C virus infection, or chronic renal or liver disease; potential antiretroviral drug interactions with other prescribed, over-the-counter, or complementary/alternative medications taken by the child; and the ability of the caregiver and child to adhere to the regimen.

Initiation of antiretroviral therapy is recommended for infants aged <12 months, regardless of clinical status, CD4 percentage, or viral load. CD4 percentage can be calculated according to methods known in the art. For example, single-platform technology (SPT), a process in which absolute counts of lymphocyte subsets are measured from a single tube by a single instrument, may be used. SPT incorporates internal calibrator beads of known quantity in the analysis of specimens by three- or four-color flow cytometry. With CD45 gating, the relative numbers of beads and lymphocyte subsets are enumerated, and their absolute numbers and percentage values are calculated [Centers for Disease Control and Prevention, Guidelines for performing single-platform absolute CD4⁺ T-cell determinations with CD45 gating for persons infected with human immunodeficiency virus MMWR 2003; 52(No. RR-2): 1-13].

Issues associated with adherence must be fully assessed and discussed with the HIV-infected infant's caregivers before therapy is initiated. Initiation of antiretroviral therapy is recommended for children age >1 year with AIDS or significant symptoms (clinical category C or most clinical category B conditions), regardless of CD4 percentage/count or plasma HIV RNA level. Initiation of antiretroviral therapy is also recommended for children age ≧1 year who have met the age-related CD4 threshold for initiating treatment (CD4 <25% for children aged 1 to <5 years and <350 cells/mm³ for children ≧5 years), regardless of symptoms or plasma HIV RNA level. Initiation of antiretroviral therapy should be considered for children age ≧1 year who are asymptomatic or have mild symptoms and have CD4 ≧25% for children aged 1 to <5 years or ≧350 cells/mm³ for children ≧5 years and have plasma HIV RNA ≧100,000 copies/mL. Initiation of antiretroviral therapy may be deferred for children age ≧1 year who are asymptomatic or have mild symptoms and who have CD4 ≧25% for children aged 1 to <5 years and ≧350 cell/mm³ for children ≧5 years and have plasma HIV RNA <100,000 copies/mL. Because the risk of disease progression slows in children age ≧1 year, the option of deferring treatment can be considered for older children. It is clear that children with clinical AIDS or significant symptoms are at high risk of disease progression and death; treatment is recommended for all such children, regardless of immunologic or virologic status. However, children age ≧1 year with mild clinical symptoms or who are asymptomatic are at lower risk of disease progression than those with more severe clinical symptoms [Working Group on Antiretroviral Therapy and Medical Management of HIV-Infected Children. Guidelines for the Use of Antiretroviral Agents in Pediatric HIV Infection. Feb. 23, 2009; pp 1-139]. Preferred regimens for initial therapy of children are known to those of skill in the art, and are outlined in the aforementioned guidelines.

The compounds described herein (anti-fibrotics, alone or together with HIV therapeutics) may be formulated in pharmaceutical compositions with a pharmaceutically acceptable excipient, carrier, or diluent. The compound or composition comprising the compound can be administered by any route that permits treatment of the disease or condition. A preferred route of administration is oral administration. Additionally, the compound or composition comprising the compound may be delivered to a patient using any standard route of administration, including parenterally, such as intravenously, intraperitoneally, intrapulmonary, subcutaneously or intramuscularly, intrathecally, transdermally, rectally, orally, nasally or by inhalation. Slow release formulations may also be prepared from the agents described herein in order to achieve a controlled release of one or more active agents in contact with the body fluids, for example in the gastro intestinal tract, and to provide a substantially constant and effective level of one or more active agents in the blood plasma. A crystal form may be embedded for this purpose in a polymer matrix of a biological degradable polymer, a water-soluble polymer or a mixture of both, and optionally suitable surfactants. Embedding can mean in this context the incorporation of micro-particles in a matrix of polymers. Controlled release formulations are also obtained through encapsulation of dispersed micro-particles or emulsified micro-droplets via known dispersion or emulsion coating technologies.

Administration may take the form of single dose administration, or the compound of the embodiments can be administered over a period of time, either in divided doses or in a continuous-release formulation or administration method (e.g., a pump). However the compounds of the embodiments are administered to the subject, the amounts of compound administered and the route of administration chosen should be selected to permit efficacious treatment of the disease condition.

In an embodiment, the pharmaceutical compositions may be formulated with pharmaceutically acceptable excipients such as carriers, solvents, stabilizers, adjuvants, diluents, etc., depending upon the particular mode of administration and dosage form. The pharmaceutical compositions should generally be formulated to achieve a physiologically compatible pH, and may range from a pH of about 3 to a pH of about 11, preferably about pH 3 to about pH 7, depending on the formulation and route of administration. In alternative embodiments, it may be preferred that the pH is adjusted to a range from about pH 5.0 to about pH 8. More particularly, the pharmaceutical compositions may comprise a therapeutically or prophylactically effective amount of at least one compound as described herein, together with one or more pharmaceutically acceptable excipients. Optionally, the pharmaceutical compositions may comprise a combination of the compounds described herein, or may include a second active ingredient useful in the treatment or prevention of bacterial infection (e.g., anti-bacterial or anti-microbial preservative agents).

Formulations for parenteral or oral administration are most typically solids, liquid solutions, emulsions or suspensions, while inhalable formulations for pulmonary administration are generally liquids or powders, with powder formulations being generally preferred. A preferred pharmaceutical composition may also be formulated as a lyophilized solid that is reconstituted with a physiologically compatible solvent prior to administration. Alternative pharmaceutical compositions may be formulated as syrups, creams, ointments, tablets, and the like.

The term “pharmaceutically acceptable excipient” refers to an excipient for administration of a pharmaceutical agent, such as the compounds described herein. The term refers to any pharmaceutical excipient that may be administered without undue toxicity.

Pharmaceutically acceptable excipients are determined in part by the particular composition being administered, as well as by the particular method used to administer the composition. Accordingly, there exists a wide variety of suitable formulations of pharmaceutical compositions (see, e.g., Remington's Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants (e.g., ascorbic acid), chelating agents (e.g., EDTA), carbohydrates (e.g., dextrin, hydroxyalkylcellulose, and/or hydroxyalkylmethylcellulose), stearic acid, liquids (e.g., oils, water, saline, glycerol and/or ethanol) wetting or emulsifying agents, pH buffering substances, binders (e.g., povidone, microcrystalline cellulose, hydroxymethyl cellulose, hydroxypropylcellulose), disintegrants (e.g., agar-agar, algins, calcium carbonate, carboxymethylcellulose, cellulose, clays, colloid silicon dioxide, croscarmellose sodium, crospovidone, gums, magnesium aluminium silicate, methylcellulose, polacrilin potassium, sodium alginate, low substituted hydroxypropylcellulose, and cross-linked polyvinylpyrrolidone hydroxypropylcellulose, sodium starch glycolate, and starch), and the like. Liposomes are also included within the definition of pharmaceutically acceptable excipients.

The pharmaceutical compositions described herein may be formulated in any form suitable for an intended method of administration. When intended for oral use for example, tablets, troches, lozenges, aqueous or oil suspensions, non-aqueous solutions, dispersible powders or granules (including micronized particles or nanoparticles), emulsions, hard or soft capsules, syrups or elixirs may be prepared. Compositions intended for oral use may be prepared according to any suitable method, including methods known to the art for the manufacture of pharmaceutical compositions, and such compositions may contain one or more agents including sweetening agents, flavoring agents, coloring agents and preserving agents, in order to provide a palatable preparation.

Pharmaceutically acceptable excipients particularly suitable for use in conjunction with tablets and capsules include, for example, inert diluents, such as celluloses, calcium or sodium carbonate, lactose, calcium or sodium phosphate; disintegrating agents, such as cross-linked povidone, maize starch, or alginic acid; binding agents, such as povidone, microcrystalline cellulose, starch, gelatin or acacia; and lubricating agents, such as magnesium stearate, stearic acid or talc.

Tablets may be uncoated or may be coated by known techniques including microencapsulation to modify release properties such as by providing delayed release and/or sustained release properties. For example, a coating can be used to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules wherein one or more active ingredients are mixed with an inert solid diluent, for example celluloses, lactose, calcium phosphate or kaolin, a binder, such as povidone and/or microcrystalline cellulose, and a disintegrant, or as soft gelatin capsules wherein one or more active ingredients are mixed with non-aqueous or oil medium, such as glycerin, propylene glycol, polyethylene glycol, peanut oil, liquid paraffin or olive oil.

In another embodiment, pharmaceutical compositions may be formulated as suspensions comprising a compound of the embodiments in admixture with at least one pharmaceutically acceptable excipient suitable for the manufacture of a suspension.

In yet another embodiment, pharmaceutical compositions may be formulated as dispersible powders and granules suitable for preparation of a suspension by the addition of suitable excipients.

Excipients suitable for use in connection with suspensions include suspending agents (e.g., sodium carboxymethylcellulose, methylcellulose, hydroxypropyl methylcellulose, sodium alginate, polyvinylpyrrolidone, gum tragacanth, gum acacia); dispersing or wetting agents (e.g., a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate)); and thickening agents (e.g., carbomer, beeswax, hard paraffin or cetyl alcohol). The suspensions may also contain one or more preservatives (e.g., acetic acid, methyl or n-propyl p-hydroxy-benzoate); one or more coloring agents; one or more flavoring agents; and one or more sweetening agents such as sucrose or saccharin.

The pharmaceutical compositions may also be in the form of oil-in water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth; naturally occurring phosphatides, such as soybean lecithin, esters or partial esters derived from fatty acids; hexitol anhydrides, such as sorbitan monooleate; and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

Additionally, the pharmaceutical compositions may be in the form of a sterile injectable preparation, such as a sterile injectable aqueous emulsion or oleaginous suspension. This emulsion or suspension may be formulated by a person of ordinary skill in the art using those suitable dispersing or wetting agents and suspending agents, including those mentioned above. The sterile injectable preparation may also be a sterile injectable solution or suspension in a non-toxic parenterally acceptable diluent or solvent, such as a solution in 1,2-propane-diol.

The sterile injectable preparation may also be prepared as a lyophilized powder. Among the acceptable vehicles and solvents that may be employed are water, Ringer's solution, and isotonic sodium chloride solution. In addition, sterile fixed oils may be employed as a solvent or suspending medium. For this purpose any bland fixed oil may be employed including synthetic mono- or diglycerides. In addition, fatty acids (e.g., oleic acid) may likewise be used in the preparation of injectables.

To obtain a stable water-soluble dose form of a pharmaceutical composition, a pharmaceutically acceptable salt of a compound described herein may be dissolved in an aqueous solution of an organic or inorganic acid, such as 0.3 M solution of succinic acid, or more preferably, citric acid. If a soluble salt form is not available, the compound may be dissolved in a suitable co-solvent or combination of co-solvents. Examples of suitable co-solvents include alcohol, propylene glycol, polyethylene glycol 300, polysorbate 80, glycerin and the like in concentrations ranging from about 0 to about 60% of the total volume. In one embodiment, one or more active compounds are dissolved in DMSO and diluted with water.

The pharmaceutical composition may also be in the form of a solution of a salt form of an active ingredient in an appropriate aqueous vehicle, such as water or isotonic saline or dextrose solution. Also contemplated are compounds which have been modified by substitutions or additions of chemical or biochemical moieties which make them more suitable for delivery (e.g., increase solubility, bioactivity, palatability, decrease adverse reactions, etc.), for example by esterification, glycosylation, PEGylation, etc.

In a preferred embodiment, the compounds described herein may be formulated for oral administration in a lipid-based formulation suitable for low solubility compounds. Lipid-based formulations can generally enhance the oral bioavailability of such compounds.

As such, a preferred pharmaceutical composition comprises a therapeutically or prophylactically effective amount of a compound described herein, together with at least one pharmaceutically acceptable excipient selected from the group consisting of medium chain fatty acids and propylene glycol esters thereof (e.g., propylene glycol esters of edible fatty acids, such as caprylic and capric fatty acids) and pharmaceutically acceptable surfactants, such as polyoxyl 40 hydrogenated castor oil.

In an alternative preferred embodiment, cyclodextrins may be added as aqueous solubility enhancers. Preferred cyclodextrins include hydroxypropyl, hydroxyethyl, glucosyl, maltosyl and maltotriosyl derivatives of α-, β-, and γ-cyclodextrin. A particularly preferred cyclodextrin solubility enhancer is hydroxypropyl-o-cyclodextrin (BPBC), which may be added to any of the above-described compositions to further improve the aqueous solubility characteristics of the compounds of the embodiments. In one embodiment, the composition comprises about 0.1% to about 20% hydroxypropyl-o-cyclodextrin, more preferably about 1% to about 15% hydroxypropyl-o-cyclodextrin, and even more preferably from about 2.5% to about 10% hydroxypropyl-o-cyclodextrin. The amount of solubility enhancer employed will depend on the amount of the compound of the invention in the composition.

The invention will be more fully understood by reference to the following examples which detail exemplary embodiments of the invention. They should not, however, be construed as limiting the scope of the invention. All citations throughout the disclosure are hereby expressly incorporated by reference.

### EXAMPLES

### Example 1

This example describes quantification methods which can be used in conjunction with the methods of the present disclosure. The following examples are quantitative methods described in the context of monkeys. Modifications to the protocols may be necessary for other mammals, including humans.

### CD4⁺ Cell Quantitation

Venous blood will be used to measure CD4⁺ cell count by flow cytometry. Tissue biopsies (LN and ileal GALT samples) are divided; one portion will be processed by immunohistochemical staining for quantitative image analysis to determine the absolute size of the total CD4⁺ cell population, and the remaining portion will be processed by flow cytometry to proportionately quantify CD4⁺ cells (total, naïve, central memory [CM], and effector memory [EM] T cells). These methods have been published elsewhere [Schacker et al., 2006, Clinical and Vaccine Immunology 13:556-60; Schacker et al., 2005, AIDS 19:2169-71; Schacker et al., 2002, J Clin Invest 110:1133-9; Schacker J Infect Dis 186:1092-7]. For flow cytometry, a portion of LN tissue and GALT will be placed on ice and CD4⁺ cells are isolated within 24 hours by gently separating the cells from surrounding tissue by using a mesh screen. One million cells are washed once in FACS wash (PBS supplemented with 0.1% sodium azide and 2% bovine serum albumin; Sigma). After aspiration of the supernatant, cells will be stained with peridin clorophylla protein-conjugated CD4, allophycocyanin conjugated CD8, phycoerythrin conjugated CD27, and fluorescein isothiocyanate-conjugated CD45RO (all BD Pharmingen), and incubated for 30 min at 4° C., followed by another wash. Cells will be fixed with 1% paraformaldahyde (Electron Microscopy Sciences) and analyzed on a FACS Calibur flow cytometer (BD Pharmingen). Lymphocytes will be gated on the basis of characteristic forward and side scatter properties, followed by separation into CD4⁺ T cells and CD8⁺ T cells on the basis of expression of CD4 and CD8. Naive T cells are classified by expression of CD27 without expression of CD45RO, as described elsewhere [Brenchley et al., 2003, Blood 101:2711-20]. CM T cells will be classified by coexpression of CD27 and CD45RO, and EM T cells are classified by lack of CD27 expression.

To quantify the total CD4⁺ T cell population in each compartment, 4-μm sections will be prepared from the fixed tissues and stained with antibody for CD4 by using either clone 1F6 (Ventana Medical Systems) or clone 4B12 (Neomarkers; Lab Vision). Images will be captured to quantify the percentage of tissue area occupied by CD4 by using PHOTOSHOP CS2, version 9.0 imaging software (ADOBE SYSTEMS, Inc.) with plug-ins from REINDEER GRAPHICS. Inc.

A second method that may be used to quantify central memory CD4⁺ cells in Peyer patches of GALT is as follows. Immunofluorescent images of sections triple labeled with antibodies against CD4, CD27, and CD45R0 are obtained and combined in PHOTOSHOP imaging software (ADOBE SYSTEMS) to unambiguously label CM cells in Peyer patches by using fixed tissue. Cells that are CD4⁺, CD27⁺, and CD45R0⁺ can be manually counted.

### Quantitation of Amount of Fibrosis

To quantify collagen in the TZ, methods similar to those previously published can be used [Schacker et al., 2002, J. Clin. Investig. 110:1133-1139]. Sections (4 μm) are stained using a modified trichrome stain to identify collagen fibers, and approximately 18 images from the TZ are captured and transported into PHOTOSHOP CS imaging software (ADOBE SYSTEMS, Inc.). Image analysis tools from REINDEER GRAPHICS, Inc. (Asheville, N.C.) may provide better sensitivity for isolating collagen fibers to quantify the percent area occupied by collagen.

While the methods presented herein can be used to practice the claimed methods, it will be understood by one of skill in the art that modifications may be made to improve quantitation of the parameters described herein.

### Example 2

This example describes the use pirfenidone in a non-human primate [Rhesus Macaque (Macaca mulatta)] model of Simian Immunodeficiency Virus (SIV) infection to show that pirfenidone inhibits fibrosis caused by viral replication in lymphatic tissues.

In this study, six animals were divided into two groups of three animals each. One group received pirfenidone for 2 weeks and then was infected with SIV MAC239. The other group did not receive pirfenidone. Pirfenidone was continued for 12 weeks following infection and then discontinued for 12 weeks (i.e., week 24) and then restarted. Thus, the pirfenidone-treated group would receive pirfenidone from week −2 to week 12 (14 weeks) and from week 24 to week 36 (12 weeks) for a total of 24 weeks. The other group of three animals did not receive pirfenidone at any time point. The dose of pirfenidone used was 200 mg/kg orally, twice daily (BID) for a total daily dose of 400 mg/kg/day. This corresponds to a human dose of approximately 6.5 mg/kg BID.

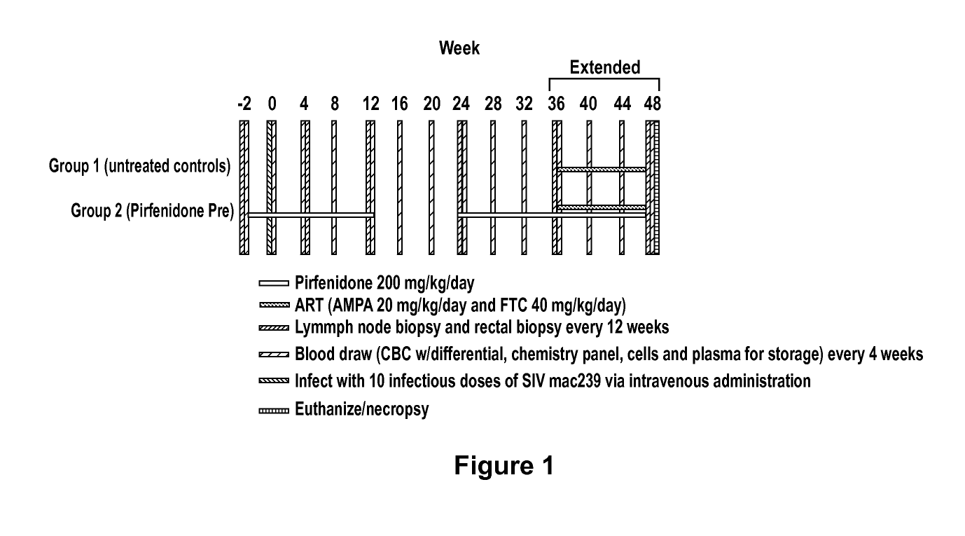

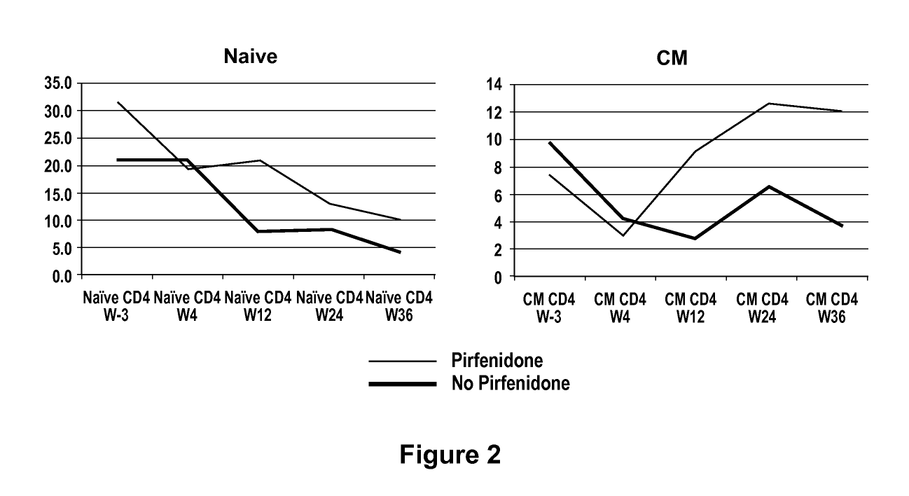

A diagram of the protocol can be seen in FIG. 1. Lymph node and rectal biopsy samples were collected at day 0 and again at weeks 4, 12, 24, and 36 after infection. Samples were analyzed by immunohistochemistry and quantitative image analysis to precisely determine the absolute size of the CD4 T cell population in the T cell zone (determined to be the percent of area that is occupied by CD4 T cells) and the degree to which the space is occupied by collagen (determined to be the percent area that is occupied by collagen). Percent of naïve versus central memory (T_{CM}) T cells were also quantitated and are shown in FIG. 2.

Pirfenidone was found to be well-tolerated without adverse effects or toxicity. One control animal (AY99) was euthanized after week 24 because of rapid progression to AIDS.

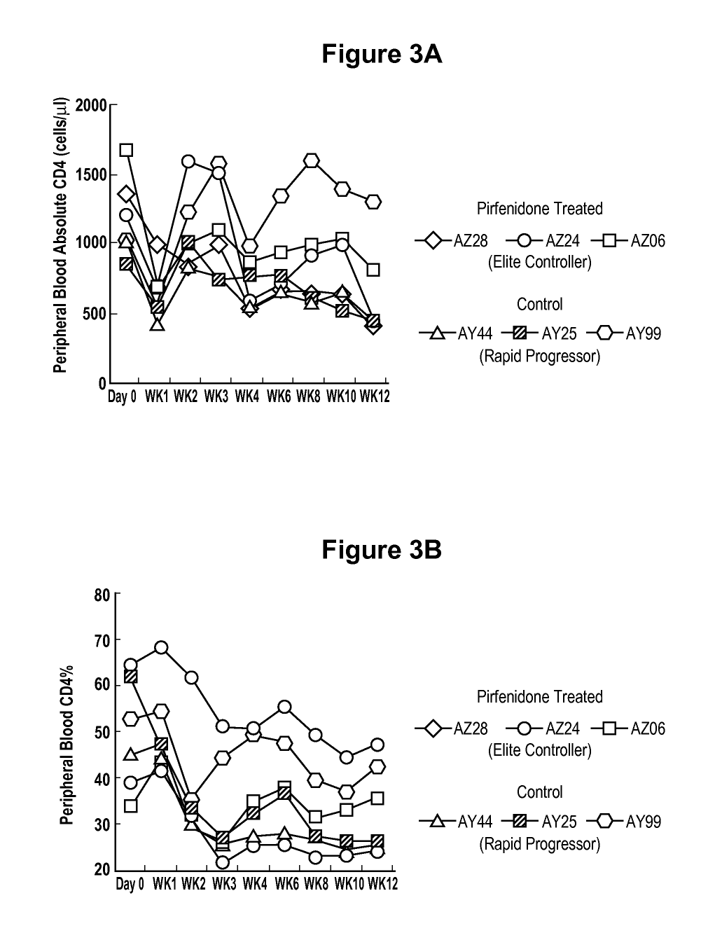

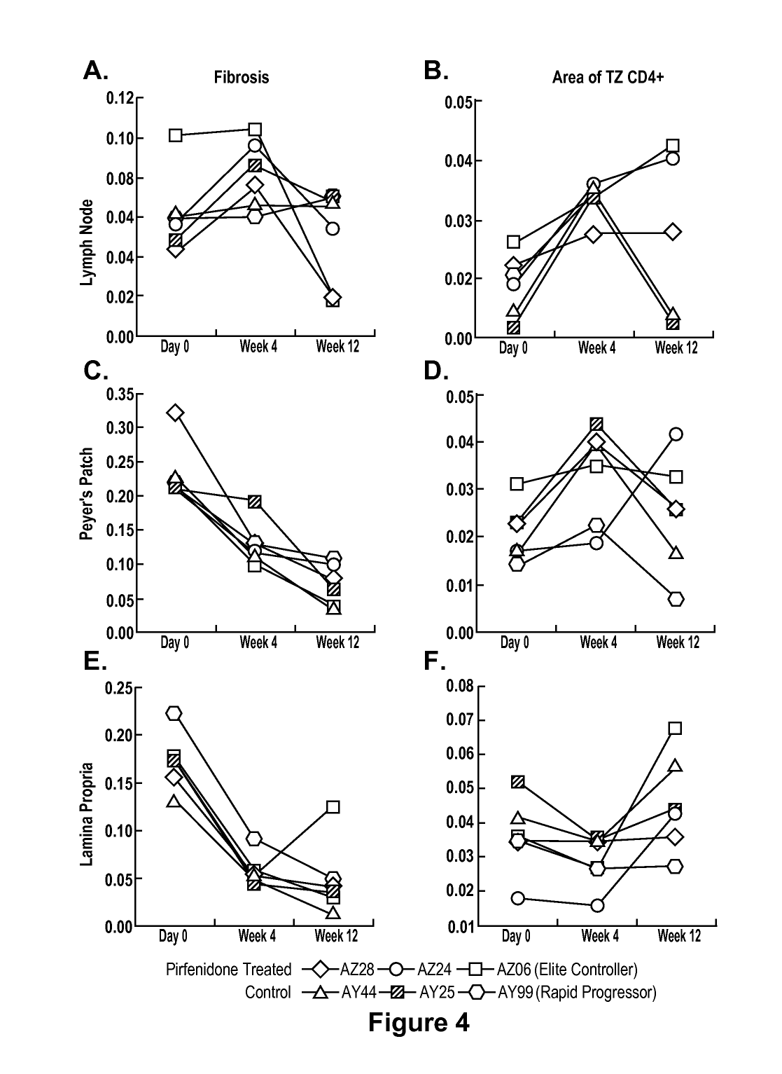

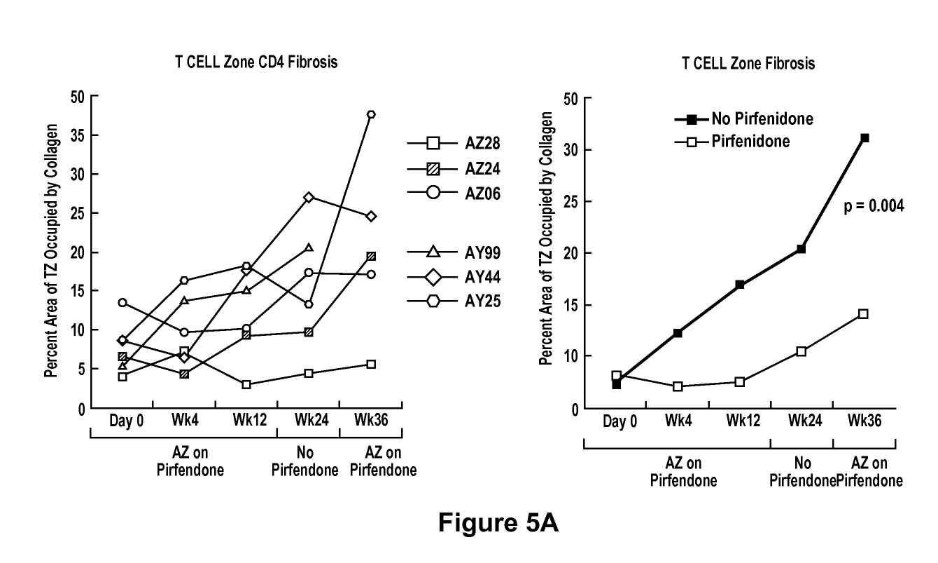

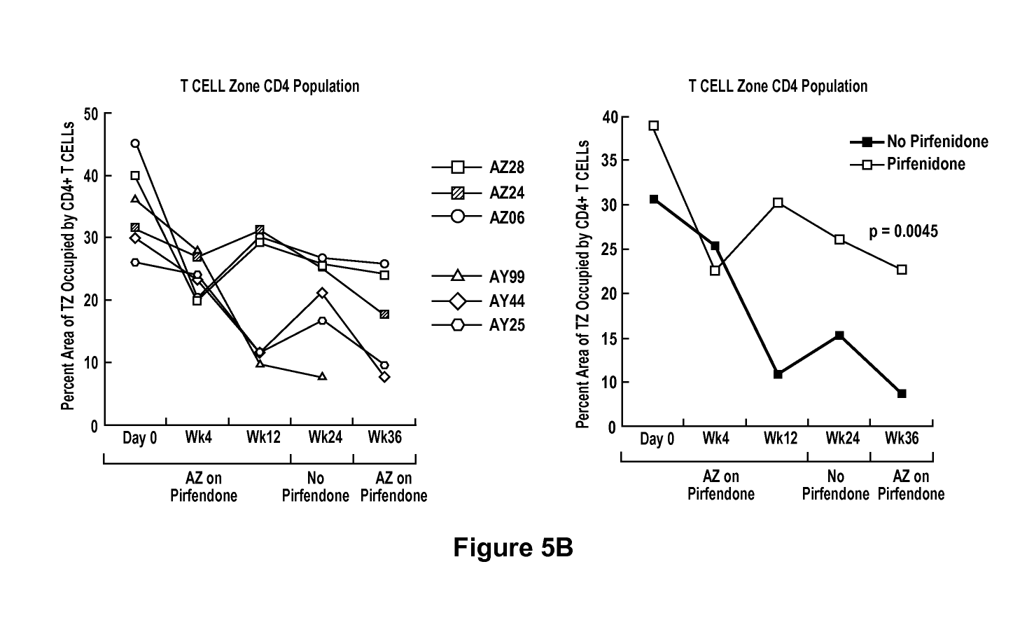

There was no significant change in peripheral blood CD4 T cell count, percent CD4, or viral load (FIG. 3). This result was expected. Fibrosis was found to be decreased and the number of CD4 cells increased in lymph nodes of animals given pirfenidone (FIGS. 4 and 5) compared to animals not given pirfenidone.

### Example 3

It is also contemplated that the study described in Example 2 may be extended to 48 weeks and beyond. The protocol for this extension is also described in FIG. 1 (shown as “Extended” in FIG. 1).

In addition to continuing the measurements described in Example 2 at the 48 week time point, both groups of animals are administered 9-[2-(R)-[[bis[[(isopropoxycarbonyl)-oxy]methoxy]phosphinoyl]methoxy]propyl]adenine fumarate (PMPA) and Emtricitabine (FTC) (i.e. antiretroviral therapy known to inhibit SIV replication) at 36 weeks after infection to observe the level of immune reconstitution in animals treated with an anti-fibrotic agent relative to untreated animals following initiation of antiretroviral therapy.

It is expected that the extension of the protocol will result in data that follow the trends described in Example 2 and that animals treated with an anti-fibrotic agent experience greater immune reconstitution upon initiation of antiretroviral therapy.

### Example 4

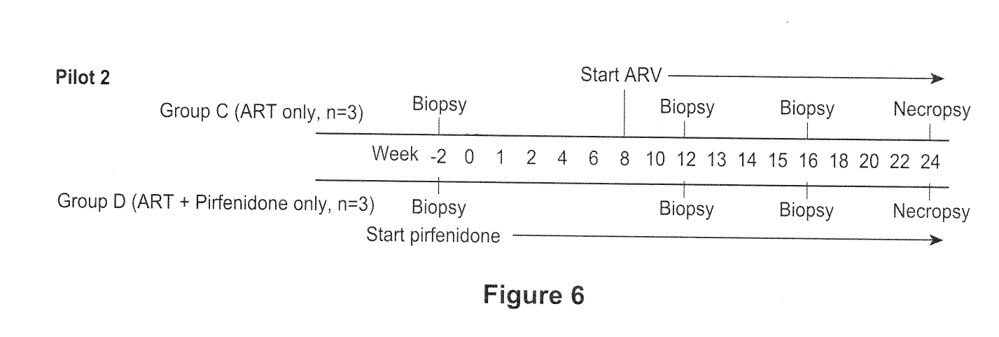

This example describes a 24-week study according to the protocol depicted in FIG. 6. In short, there were two groups in the study. The first group (group C as depicted in FIG. 6) received only ARV beginning at Week 8 (relative to SIV infection) of the study and continuing through week 24. The second group (group D as depicted in FIG. 6) received pirfenidone beginning at week −2 of the study and ARV beginning at Week 8. Both pirfenidone and ARV were then co-administered through Week 24 of the study. Tissues were biopsied at Week −2. Additional biopsies were conducted at Weeks 12, 16 and 24 (after sacrifice of the animal).

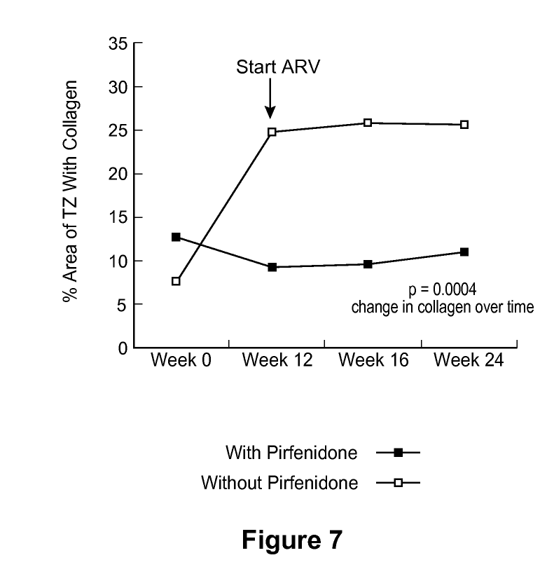

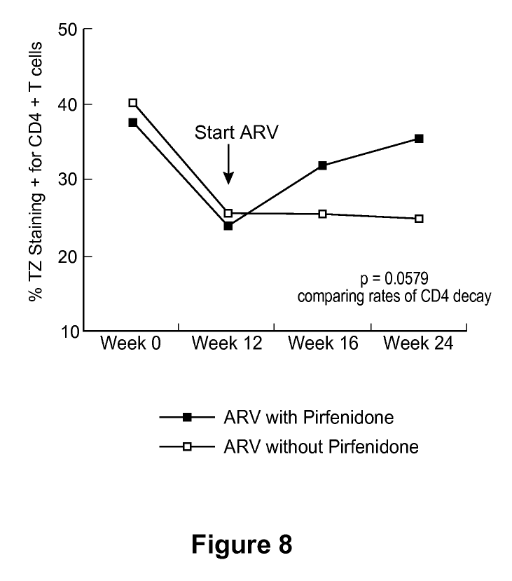

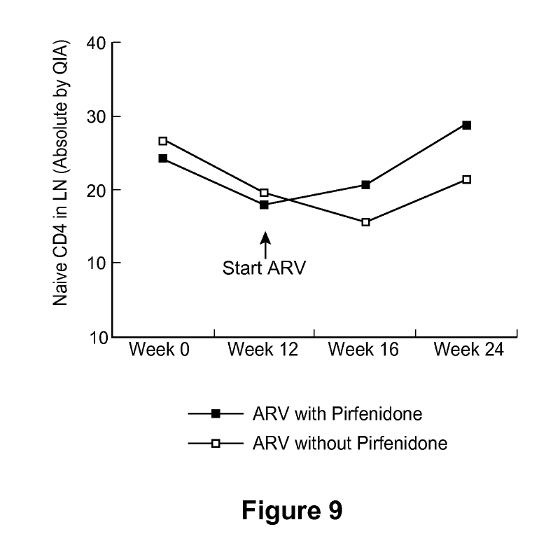

Results indicated that the percentage area of the T cell zone with collagen was significantly lower in group D relative to group C (FIG. 7). Additionally it was seen that the number of CD4 T cells in the TZ was increased starting at week 16 and continuing through week 24 in group D relative to group C (FIG. 8). This corresponded with an increase in the absolute number of naïve CD4 T cells of the TZ starting at week 16 and continuing through week 24 in group D relative to group C (FIG. 9).

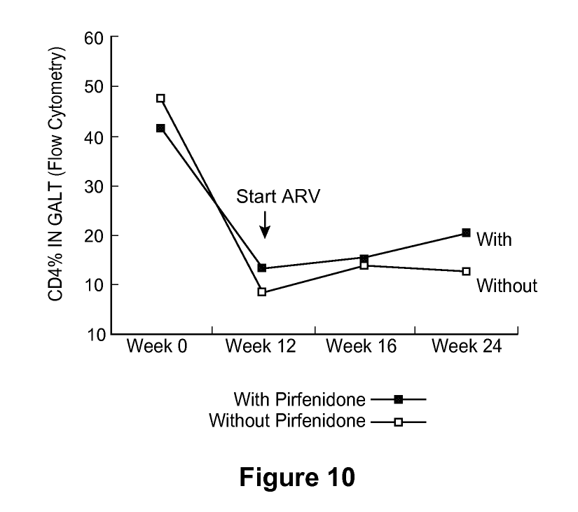

Finally, it was noted that the percentage of CD4 T cells in GALT was increased at week 24 in group D relative to group C (FIG. 10).

### Example 5

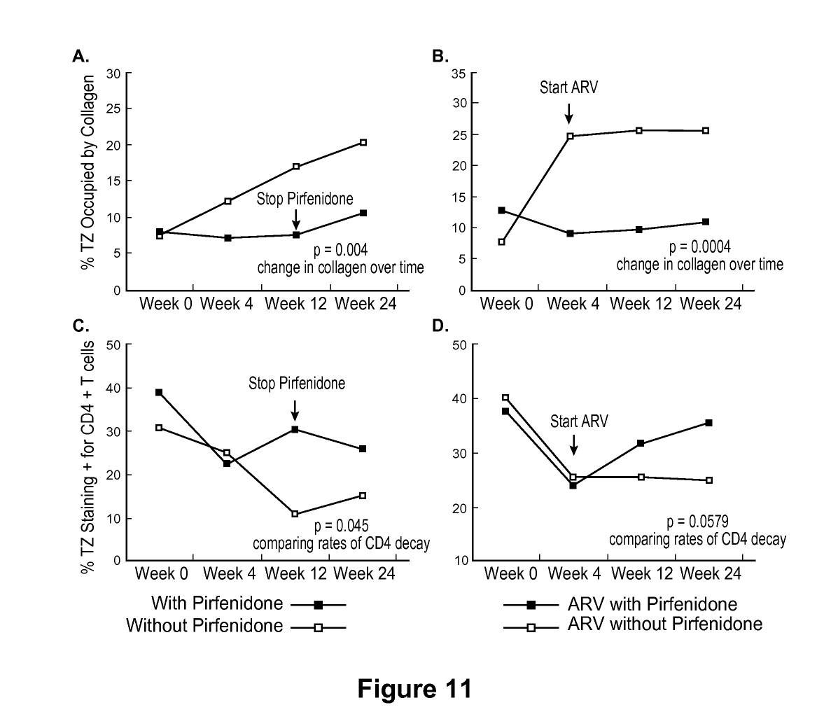

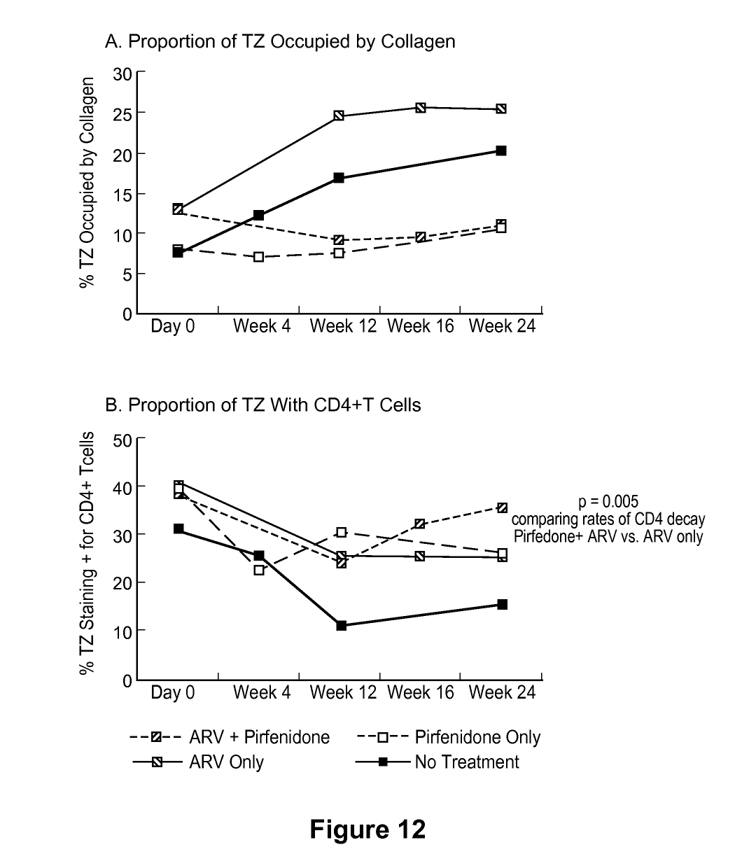

This Example provides composite representations of the studies described above. FIG. 11 depicts a significant reduction in the percentage of the TZ occupied by collagen in the protocols depicted in FIGS. 1 and 6. FIG. 11 also shows that more of the TZ stains positive for CD4⁺ T cells when pirfenidone is administered, and this result is consistent across both protocols (FIG. 11, C&D). Further, upon initiation of ARV the animals treated with pirfenidone show a greater increase in the TZ area staining positive for CD4⁺ T cells (FIG. 11D). FIG. 12 shows that the proportion of the TZ occupied by collagen is lower in the two pirfenidone treatment arms (FIG. 12A). FIG. 12A also indicates that ARV alone may not be sufficient to halt the ongoing fibrotic process. Thus, the established fibrotic process may behave independently of viral load. Accordingly, the results provide support for a conclusion that an antifibrotic is useful even in the presence of ARV-based viral suppression. Finally, FIG. 12B indicates that the co-administration of pirfenidone and ARV improves the rate of change in CD4⁺ area in TZ relative to ARV alone.

What is claimed:

1. A method of treating a patient diagnosed with human immunodeficiency virus (HIV) comprising the step of administering to said patient a therapeutically effective amount of an anti-fibrotic agent, wherein the anti-fibrotic agent optionally is pirfenidone, a pirfenidone analog or a compound of formula (I), (II), (III), (IV), or (V): 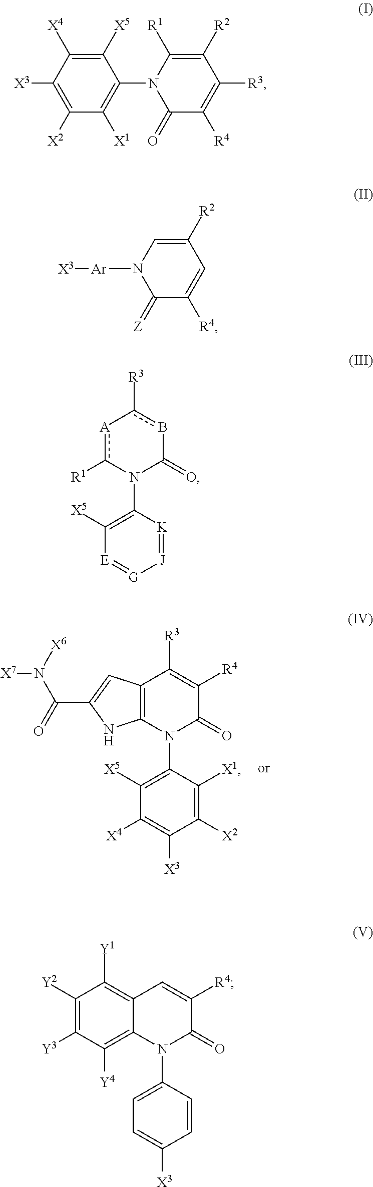
wherein
R¹, R², R³, R⁴, X¹, X², X³, X⁴, X⁵, Y¹, Y², Y³ and Y⁴ are independently selected from the group consisting of H, deuterium, C₁-C₁₀ alkyl, C₁-C₁₀ deuterated alkyl, substituted C₁-C₁₀ alkyl, C₁-C₁₀ alkenyl, substituted C₁-C₁₀ alkenyl, C₁-C₁₀ thioalkyl, C₁-C₁₀ alkoxy, substituted alkoxy, cycloalkyl, substituted cycloalkyl, heterocycloalkyl, substituted heterocycloalkyl, heteroalkyl, substituted heteroalkyl, aryl, substituted aryl, heteroaryl, substituted heteroaryl, halogen, hydroxyl, C₁-C₁₀ alkoxyalkyl, C₁-C₁₀ carboxy, C₁-C₁₀ alkoxycarbonyl, CO-uronide, CO-monosaccharide, CO-oligosaccharide, and CO-polysaccharide;
X⁶ and X⁷ are independently selected from the group consisting of hydrogen, aryl, substituted aryl, heteroaryl, substituted heteroaryl, cycloalkyl, substituted cycloalkyl, heterocycloalkyl, substituted heterocycloalkyl, alkylenylaryl, alkylenylheteroaryl, alkylenylheterocycloalkyl, alkylenylcycloalkyl, or X⁶ and X⁷ together form an optionally substituted 5 or 6 membered heterocyclic ring; and
Ar is pyridinyl or phenyl; and Z is O or S;
and a HIV therapeutic agent, said amount of anti-fibrotic agent effective to decrease fibrosis in a T cell zone (TZ) of lymphatic tissue in said patient relative to a patient that is not treated with said anti-fibrotic agent, and said amount of the HIV therapeutic agent effective to increase CD4⁺ T cells in said patient relative to a patient that is not treated with said HIV therapeutic agent.

2. The method of claim 1 comprising decreasing the fibrosis by at least 5%.

3. The method of claim 1 comprising increasing CD4⁺ T cell count by at least 5% in the area of the TZ.

4. The method of claim 1 comprising increasing CD4⁺ T cell count in peripheral blood by about 10 cells per mm³.

5. The method of claim 1 wherein the amount of HIV therapeutic agent administered is a reduced amount relative to the amount indicated for administration to the patient in the absence of said anti-fibrotic agent.

6. The method of claim 1 comprising concurrently administering said anti-fibrotic agent with a HIV therapeutic agent.

7. The method of claim 1 comprising commencing administration of the anti-fibrotic agent while the patient has a T cell count of at least about 350 cells/mm³.

8. The method of claim 1 comprising commencing the administrating of said HIV therapeutic agent while the patient has a T cell count of at least about 350 cells/mm³.

9. The method of claim 7 comprising commencing the administrating of said HIV therapeutic agent while the patient has a T cell count of at least about 350 cells/mm³.

10. The method of claim 1 comprising commencing administration of the anti-fibrotic agent while the patient has a T cell count of less than about 350 cells/mm³.

11. A method of treating a patient diagnosed with HIV comprising: administering to said patient a therapeutically effective amount of an anti-fibrotic agent, wherein the anti-fibrotic agent optionally is pirfenidone, a pirfenidone analog or a compound of formula (I), (II), (III), (IV), or (V): 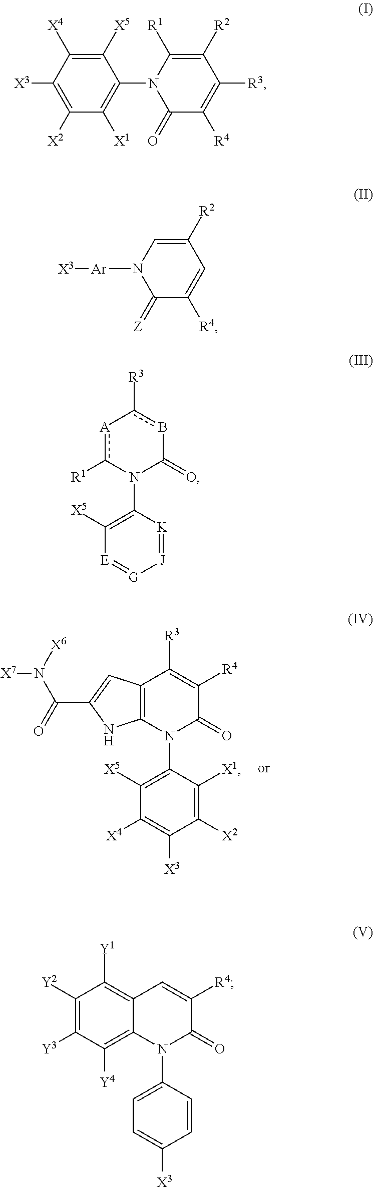
wherein
R¹, R², R³, R⁴, X¹, X², X³, X⁴, X⁵, Y¹, Y², Y³ and Y⁴ are independently selected from the group consisting of H, deuterium, C₁-C₁₀ alkyl, C₁-C₁₀ deuterated alkyl, substituted C₁-C₁₀ alkyl, C₁-C₁₀ alkenyl, substituted C₁-C₁₀ alkenyl, C₁-C₁₀ thioalkyl, C₁-C₁₀ alkoxy, substituted alkoxy, cycloalkyl, substituted cycloalkyl, heterocycloalkyl, substituted heterocycloalkyl, heteroalkyl, substituted heteroalkyl, aryl, substituted aryl, heteroaryl, substituted heteroaryl, halogen, hydroxyl, C₁-C₁₀ alkoxyalkyl, C₁-C₁₀ carboxy, C₁-C₁₀ alkoxycarbonyl, CO-uronide, CO-monosaccharide, CO-oligosaccharide, and CO-polysaccharide;
X⁶ and X⁷ are independently selected from the group consisting of hydrogen, aryl, substituted aryl, heteroaryl, substituted heteroaryl, cycloalkyl, substituted cycloalkyl, heterocycloalkyl, substituted heterocycloalkyl, alkylenylaryl, alkylenylheteroaryl, alkylenylheterocycloalkyl, alkylenylcycloalkyl, or X⁶ and X⁷ together form an optionally substituted 5 or 6 membered heterocyclic ring; and
Ar is pyridinyl or phenyl; and Z is O or S;
in the absence of a HIV therapeutic agent for a first period of time; and
administering a therapeutically effective amount of HIV therapeutic agent in the absence of administration of the anti-fibrotic agent for a second period of time following the first period of time.

12. The method of claim 7, further comprising administering an anti-fibrotic agent in combination with an HIV therapeutic agent for a third, intermediate, period of time following said first period of time and prior to said second period of time.

13. A method of treating a patient diagnosed with HIV comprising: administering to said patient a therapeutically effective amount of an anti-fibrotic agent, wherein the anti-fibrotic agent optionally is pirfenidone, a pirfenidone analog or a compound of formula (I), (II), (III), (IV), or (V): 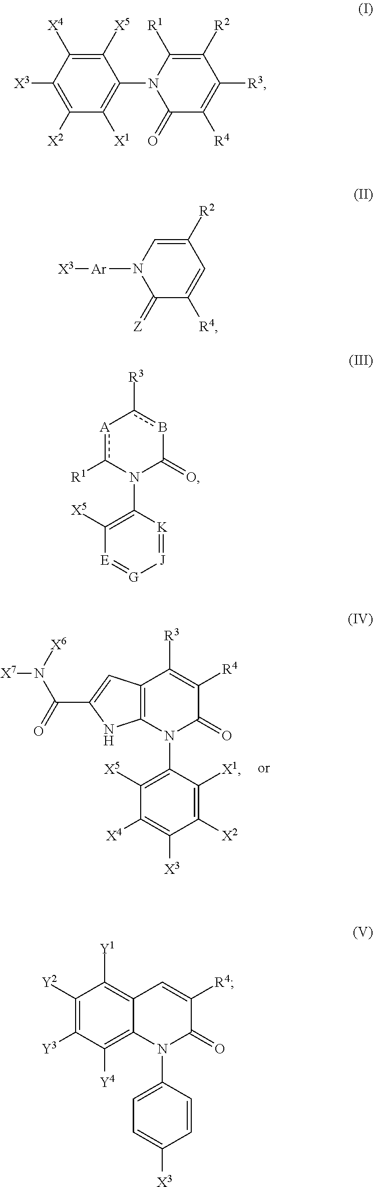
wherein
R¹, R², R³, R⁴, X¹, X², X³, X⁴, X⁵, Y¹, Y², Y³ and Y⁴ are independently selected from the group consisting of H, deuterium, C₁-C₁₀ alkyl, C₁-C₁₀ deuterated alkyl, substituted C₁-C₁₀ alkyl, C₁-C₁₀ alkenyl, substituted C₁-C₁₀ alkenyl, C₁-C₁₀ thioalkyl, C₁-C₁₀ alkoxy, substituted alkoxy, cycloalkyl, substituted cycloalkyl, heterocycloalkyl, substituted heterocycloalkyl, heteroalkyl, substituted heteroalkyl, aryl, substituted aryl, heteroaryl, substituted heteroaryl, halogen, hydroxyl, C₁-C₁₀ alkoxyalkyl, C₁-C₁₀ carboxy, C₁-C₁₀ alkoxycarbonyl, CO-uronide, CO-monosaccharide, CO-oligosaccharide, and CO-polysaccharide;
X⁶ and X⁷ are independently selected from the group consisting of hydrogen, aryl, substituted aryl, heteroaryl, substituted heteroaryl, cycloalkyl, substituted cycloalkyl, heterocycloalkyl, substituted heterocycloalkyl, alkylenylaryl, alkylenylheteroaryl, alkylenylheterocycloalkyl, alkylenylcycloalkyl, or X⁶ and X⁷ together form an optionally substituted 5 or 6 membered heterocyclic ring; and
Ar is pyridinyl or phenyl; and Z is O or S;
in the absence of a HIV therapeutic agent.

14. The method of claim 13 comprising alternating administration to said patient of therapeutically effective amounts of (a) the anti-fibrotic agent and (b) the HIV therapeutic agent.

15. The method of claim 13 comprising commencing administration of the anti-fibrotic agent while the patient has a T cell count of at least about 350 cells/mm³.

16. The method of claim 13 comprising commencing the administrating of said HIV therapeutic agent while the patient has a T cell count of at least about 350 cells/mm³.

17. The method of claim 15 comprising commencing the administrating of said HIV therapeutic agent while the patient has a T cell count of at least about 350 cells/mm³.

18. The method of claim 13 comprising commencing administration of the anti-fibrotic agent while the patient has a T cell count of less than about 350 cells/mm³.

19. The method of claim 1 wherein the administration of the anti-fibrotic agent is commenced at the time the patient is diagnosed with HIV.

20. The method of claim 1 wherein the HIV therapeutic agent is antiretroviral therapy.
